(19) United States
(12) Patent Application Publication
(10) Pub. No.: US 2014/0219925 A1
(43) Pub. Date: Aug. 7, 2014

(54) POLYMER PARTICLES OR NANO-VECTORS AND USE THEREOF AS A DRUG AND/OR DIAGNOSTIC AGENT

(71) Applicants: Philippe Bertrand, Aslonnes (FR); Regis Delatouche, L'Houmeau (FR); Valerie Heroguez, Merignac (FR); Floraine Collette, Biganos (FR); Marc Gregoire, Nantes (FR); Christophe Blanquart, Le pellerin (FR); Fabien Gueugnon, SAINT-Philibert De Bouaine (FR)

(72) Inventors: Philippe Bertrand, Aslonnes (FR); Regis Delatouche, L'Houmeau (FR); Valerie Heroguez, Merignac (FR); Floraine Collette, Biganos (FR); Marc Gregoire, Nantes (FR); Christophe Blanquart, Le pellerin (FR); Fabien Gueugnon, SAINT-Philibert De Bouaine (FR)

(73) Assignee: INSERM (Institut National de la Sante et de la Recherche Medicale), PARIS CEDEX 13 (FR)

(21) Appl. No.: 14/129,164

(22) PCT Filed: Jun. 28, 2012
(86) PCT No.: PCT/FR2012/051498

§ 371 Date: Feb. 12, 2014

(30) Foreign Application Priority Data
Jun. 28, 2011 (FR) 11/55761

(51) Int. Cl. A61K 47/48; A61K 49/00

(52) U.S. Cl. CPC A61K 47/48215; A61K 49/0054
USPC 424/9.6; 548/255; 514/359; 548/256

(57) ABSTRACT

Novel polymer nanovectors or particles and use thereof as medication and/or diagnostic agents.

The present invention relates to polymer nanovectors or particles and use thereof as medication and/or diagnostic agent.

The targeting of therapeutic agents, whatever their fields of application, is still a challenge. The direct administration of an active molecule generally comes up against the possibility of diffusion outside the desired treatment zone.

In the case of molecules that are to act at the level of tumour cells, there is the problem of distinguishing between healthy cells and abnormal cells. Defence mechanisms against exogenous agents exist within cells and can cause elimination of these therapeutic agents and can lead to phenomena of resistance of certain cancerous lines. These problems of targeting have been investigated in many works. The various strategies developed can be summarized by two approaches: active targeting and passive targeting. Active targeting is based on specific interaction between the agent and the cell, based in particular on the use of ligand-receptor or antigen-antibody pairs. Passive targeting aims to increase the quantities of agents delivered by using the physiological properties of the targets. Long regarded as giving poorer results than active targeting, this approach has undergone recent development as a result of the new strategy proposed by Maeda in 1986 (Maeda H., Matsumara Y., Cancer Res., 1986, 46, 6387-92), and based on the concept of enhanced permeability and retention (EPR) that is characteristic of tumours.

The systems developed must have three essential properties: be inactive for the time of transport, allow targeting to the desired treatment zone and have a vector that releases the drugs once this zone is reached.

In the particular case of antitumour agents, the treatment zone is in this case the cell and the drug is guided on the basis of differences that exist between healthy cells and tumour cells, principally by identifying proteins that are overexpressed on the surface of the latter (Papot S., Tranoy I., Tillequin F., Florent J.-C., Gesson J.-P., Curr. Med. Chem., Anti-Cancer Agents, 2002, 2, 155). The most advanced compounds obtained on this principle are in phase I/II and Mylotarg® is one of the rare examples used in clinical practice (Wu A. M., Senter P. D., Nature Biotechnology, 2005, 23 (9), 1137-1146).

This approach has some drawbacks. Recognition based on surface receptors overexpressed in tumour cells is not completely selective for the tumours. The kinetics of release of the drug may be slow and, once released, it must cross the cell membrane quickly to avoid being transported away from the vicinity of the tumour.

Moreover, once internalized by the cell, the molecule must find its target, which can be cytoplasmic or nuclear. In order to overcome these various drawbacks, the need has arisen for directed targeting, followed by cell penetration and ending with release of the drug in the cell. In this new approach, the concept of guiding to the tumour cells is still important and possible spread to unintended zones must be avoided.

The numerous studies carried out for active targeting of antitumour agents are now joined by the design of systems intended for passive targeting, taking advantage of the physiological differences between healthy cells and tumour cells.

The anarchic development of tumours induces an inhomogeneous structure having considerable vascularization that is very permeable due to greater spacing of the endothelial cells. Moreover, not having an effective drainage system, tumours accumulate external elements more easily. These two characters allow, among other things, increased supply of nutrients necessary for the rapid development of these cells and also promote angiogenesis. This phenomenon has been called enhanced permeability and retention (EPR) and its exploitation was proposed by Maeda and Matsumara.

This leads to preferential extravasation and to the retention of molecules of high molecular weight: the higher the latter (>40 kDa), the slower the circulation and the more the molecule can be trapped by EPR. Conversely, therapeutic agents of low molecular weight can be disseminated by circulation and diffusion (Fréchet J. M. J., Gillies E. R., Goodwin A. P., Bioconjugate Chem., 2004, 15, 1254; Patel V. F., Hardin J. N., Mastro J. M., Luw K. L., Zimmermann J. L., Ehlhardt W. J., Woodland J. M., Starling J. J., Bioconjugate Chem., 1996, 7(4), 497; Ulbrich K., Subr V., Adv. Drug Delivery Rev., 2004, 56, 1023).

At the level of the eukaryotic cell, the passage of small molecules through membranes takes place by natural diffusion. The passage of macromolecules through this membrane normally takes place by a mechanism called endocytosis. In this process, the macromolecules interact with receptors on the cell surface, causing a change in the membrane that encloses the structure and, by internal detachment, produces an organelle that is generally called an endosome. During their formation at the level of the cell membrane, these endosomes quickly reach an internal pH close to 6. By a process of maturation, this pH is gradually lowered, with perinuclear displacement, the endosomes finally binding to the primary lysosomes, giving an assembly called secondary lysosomes with a more acidic internal pH close to 4-5, which is a reservoir of hydrolases the function of which is to degrade the incorporated molecules into their simplest elements (amino acids, nucleosides, etc.). These elements are then released via specific channels into the cytoplasm, where they can be used. Three routes of endocytosis have been described so far (Kirkham M., Parton R. O., Biochimica et Biophysica Acta, 2005, 1745, 273-286).

Among the systems proposed previously in the literature for releasing a molecule in the cell (V. F. Patel, J. N. Hardin, J. M. Mastro, K. L. Law, J. L. Zimmermann, W. J. Ehlhardt, J. M. Woodland, J. J. Starling, Bioconjugate Chem. 1996, 7, 497; E. Leikauf, F. Barnekow, H. Köster, Tetrahedron 1995, 51, 3793), the trityl unit has been exploited. It allows the preparation of acid-sensitive prodrugs only for active ingredients having alcohols or amines.

The membrane barrier can be altered by various processes, in particular under the action of viruses. In 1988, two teams, that of Green and Lowenstein (Green M., Loewenstein P. M., Cell, 1988, 55, 1179) and that of Frankel and Pabo (Frankel A. D., Pabo C. O., Cell, 1988, 55, 1189) demonstrated the ability of the TAT protein of HIV-1 to cross the cell membrane. This observation led to many studies, in particular aiming to ascertain the minimum sequence of amino acids necessary for said passage or to isolate other proteins having the same properties. A report relating to the TAT protein has been published (Dowding S. F., Wading J. S., Advanced Drug Delivery Reviews, 2005, 57, 579) in parallel with a general review of cell-penetrating peptides (CPPs) with a mechanistic model of this internalization (Zorko M., Langke V., Advanced Drug Delivery Reviews, 2005, 57, 529).

From the various works presented, it is clear that although internalization by the use of these peptides is effective and widespread, its mechanism has not yet been fully established. A common point seems to be interactions of surfaces with proteoglycans which trigger the mechanism. Depending on the peptides or the concentration on the cell surface, this internalization can be carried out by classical endocytosis, by endocytosis activated by lipid displacement (Foerg C., Zieglr V., Fernandez-Carneado J., Giral E., Rennert R., Beck-Sickinger A. O., Merkle H. P., Biochemistry, 2005, 44, 72), or by routes not using endocytosis and not yet determined (Thoren P. E. O., Persson D., Lincoln P. Norden B., Biophysical Chemistry, 2005, 114, 169). Study of the TAT protein has shown that the latter evades lysosomal degradation but can be retained in the endosomes (Caron N. J., Quenneville S. P., Tremblay J. P. Biochemical and Biophysical Research Communications, 2004, 319, 12). Moreover, vector polymers have been developed that perturb the stability of the endosomes, either by causing the latter to burst (Bulmus V., Woodward M., Lin L., Murthy N., Stayton P., Hoffman A., Journal of Controled Release, 2003, 93, 105), or by modifying the internal pH, such as in the case of (nitrogen-rich) cationic polymers such as polylysines or imidazole-modified polylysines (Putnam D., Gentry C. A., Pack D. W., Langer R., Proc. Natl. Acad. Sci., 2001, 98 (3), 1200; Merdan T., Kopecek J., Kissel T., Advanced Drug Delivery Reviews, 2002, 54, 715). Utilized for example in DNA transport, these polymers also produce interactions of charges that give compact complexes that are more stable vis-à-vis possible degradations.

Molecular objects have thus been designed of sufficient size so that they circulate slowly in the body and avoid renal elimination (size greater than 40 kDa) or trapping by the reticuloendothelial system (size greater than 200 kDa).

A ratio must be found between the molecular weight of the system and its effective volume. Two types of applications have in particular been developed: biocompatible polymers (Pluronic®) (Kabanov A. V., Batrakova E. V., Alakhov V. Y., Adv. Drug. Dev. Reviews, 2002, 54, 759-779) and liposomes (Doxil®=Polyethylene glycol-liposome doxorubicin (Ceh B., Winterhalter M., Frederik P. M., Vallner J. J., Lasie D. D., Adv. Drug. Dev. Reviews, 1997, 24, 165-177)). One important aspect of these systems is their ability to greatly reduce the phenomena of resistance.

Although passive targeting has in itself demonstrated therapeutic improvement for the various molecules transported (solubility, toxicity), it is necessary to develop systems the vector of which can be eliminated easily with characteristics suitable for endocytosis and capable of releasing the drug in the cell, and in particular in acidic organelles such as endosomes (pH 6) or lysosomes (pH 5), in particular in the cancer cell, thus making it possible to avoid the severe side-effects observed owing to non-selective action of said molecules on cells other than cancer cells.

Moreover, application WO 2006/008387 describes polymer particles that can be stimulated in particular in an acid environment, having reactive functions but with the major drawback of not being able to bear reactive functions that are on the one hand sensitive to the method for obtaining said particles and on the other hand require the development of a specific synthesis for each reactive function to be used.

One aspect of the invention is to supply nanovectors, in the form of polymer or not, comprising at least one active ingredient, in particular an epigenetic modulator, and/or at least one detecting probe and/or a cell-penetrating peptide, said nanovectors being capable of penetrating into a cell, in particular a cancer cell.

Another aspect of the invention is to use said nanovectors as a medicament, in particular an anticancer and/or diagnostic agent.

A third aspect of the invention is to provide pharmaceutical compositions comprising said nanovectors.

A final aspect of the invention is to provide methods for the synthesis of said nanovectors that can be used irrespective of the epigenetic modulator or detecting probe present on the nanovector.

The present invention relates to nanovectors constituted by polymer chains Pi of the following general formula (I):

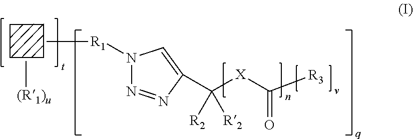

in which:

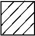

represents a polymer chain P, in particular a polymer chain P containing about 30 to 10,000 monomer units, identical or different, derived from the polymerization of monocyclic alkenes in which the number of carbon atoms constituting the ring is from about 4 to 12, or of polycyclic alkenes in which the total number of carbon atoms constituting the rings is from about 6 to 20,
t represents 0 or 1,
q is an integer in the range from 1 to 10,
u represents an integer from 0 to 10,
n represents 0 or 1,
v represents 0 or 1,
X represents O, NH or S,
R₁ and R′₁ represent, independently of one another, when t=1, a group of the following Formula (II):

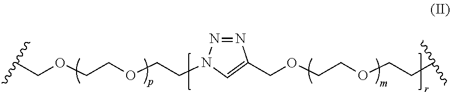

where:
   m and p represent, independently of one another, an integer from 1 to 1000, in particular 50 to 340, in particular 70 to 200
   r is an integer in the range from 0 to 10, preferably 0 or 1,
or,
R₁ represents, when t=0, a group of the following Formula (III) linked to a monocyclic alkene or a polycyclic alkene:

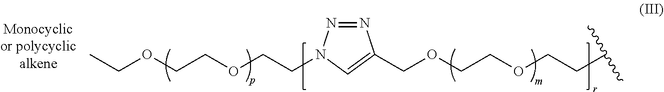

in which the number of carbon atoms constituting the ring of the monocyclic alkene is from about 4 to 12, and the total number of carbon atoms constituting the rings of the polycyclic alkene is from about 6 to 20,
r, m and p being as defined above,
or,
R₁ represents, when t=0, a group of the following Formula (IV):

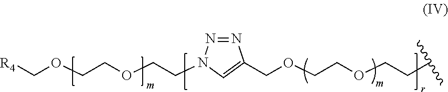

in which R₄ represents: a vinyl group, an ethyne group, an OR′ or SR″ group, R′ and R″ representing, independently of one another, H, a C₁-C₂₀ alkyl, a C₃-C₂₀ cycloalkyl, and m being as defined above,
r is an integer in the range from 0 to 10, preferably 0,
R₂ and R′₂ represent, independently of one another:
   H or a phenyl, unsubstituted or substituted by at least:
      a C₁-C₂₀ alkyl, a C₃-C₂₀ cycloalkyl,
      a C₁-C₂₀ alkoxy,
      NRₐR_{b} where Rₐ and R_{b} represent, independently of one another, H, a C₁-C₂₀ alkyl, the alkyl being able to form a ring with the carbon or carbons ortho to that bearing NRₐR_{b}, a C₃-C₂₀ cycloalkyl,
      NO₂,
      CO₂Rc, where Rc represents H, a C₁-C₂₀ alkyl, a C₃-C₂₀ cycloalkyl, a substituted or unsubstituted benzyl,
      a C₁-C₂₀ acyl,
   in particular R₂ and R′₂ represent 2- or 4-methoxyphenyl, 2- or 4-methylphenyl, phenyl, 2,4-dimethoxyphenyl, and when n=0 and v=1, R₃ is then bound directly to the carbon bearing R₂ and R′₂,
or,
R₂ and R′₂ represent together, if n=0 and v=0, the ring of the following Formula (Va):

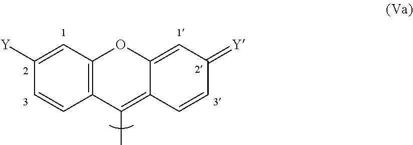

in which Y′ represents:
   O,
   NR_{d}Rₑ where R_{d} and Rₑ represent, independently of one another, H, a C₁-C₂₀ alkyl, the alkyl being able to form a ring with carbon 1′ or 3′, a C₃-C₂₀ cycloalkyl, the nitrogen atom having a positive charge associated with a monovalent anion,
and Y represents
   OR′, where R′ represents H, a C₁-C₂₀ alkyl, a C₃-C₂₀ cycloalkyl,
   a C₁-C₂₀ alkyl, a C₃-C₂₀ cycloalkyl,
   a C₁-C₂₀ alkoxy,
   NR_{f}R_{g} where R_{f} and R_{g} represent, independently of one another, H, a C₁-C₂₀ alkyl, the alkyl being able to form a ring with carbon 1 or 3, a C₃-C₂₀ cycloalkyl,
   NO₂,
   CO₂Rc, where Rc represents H, a C₁-C₂₀ alkyl, a C₃-C₂₀ cycloalkyl, a substituted or unsubstituted benzyl,
   a C₁-C₂₀ acyl,
or, if n=0 and v=0, the ring of the following Formula (Vaa):

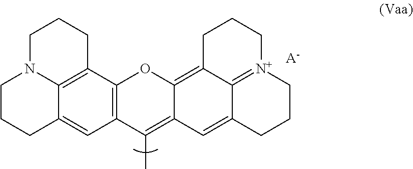

in which A⁻ represents a monovalent anion,
   or
R₂ and R′₂ together represent the ring of the following Formula (Vb), n=1 and v=1:

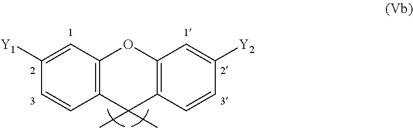

and Y₁ and Y₂ represent, independently of one another:
   OR′, where R′ represents H, a C₁-C₂₀ alkyl, a C₃-C₂₀ cycloalkyl,
   a C₁-C₂₀ alkyl, a C₃-C₂₀ cycloalkyl,
   a C₁-C₂₀ alkoxy,
   NRₕRᵢ where Rₕ and Rᵢ represent, independently of one another, H, a C₁-C₂₀ alkyl, the alkyl being able to form a ring with carbon 1 or 3 in the case of Y₁, and carbon 1′ or 3′ in the case of Y₂, a C₃-C₂₀ cycloalkyl,
   NO₂,
   CO₂Rc, where Rc represents H, a C₁-C₂₀ alkyl, a C₃-C₂₀ cycloalkyl, a substituted or unsubstituted benzyl,
   a C₁-C₂₀ acyl,
or the ring of the following Formula (Vbb) and n=1:

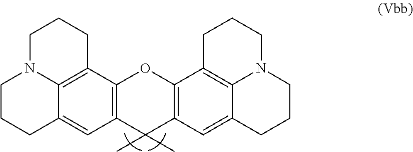

R₃ represents an active ingredient, in particular an epigenetic modulator, or a detecting probe, in particular fluorescent or radio-emitting, or a cell-penetrating peptide (CPP),

By the term “polymer chain P” is meant a substance composed of a large number of small molecular structures of low mass, identical or different, which link together, in a chain or in a network, to create molecules having a high molecular weight.

The polymer chain P contains monomer units derived from the polymerization of monocyclic or polycyclic alkenes. It can be of the polynorbornene type.

In particular, the polymer chain P contains from about 30 to 10,000 monomer units, identical or different, in particular 340 units.

In particular, said monomer units are derived from the polymerization of monocyclic alkenes constituted by 4 to 12 carbons or of polycyclic alkenes constituted by 6 to 20 carbons.

The expression “t represents 0 or 1” means that the polymer chain P can be present or not.

When t=0, the polymer chain P is not present in general formula (I). Said general formula (I) therefore corresponds to the following Formula (I-a):

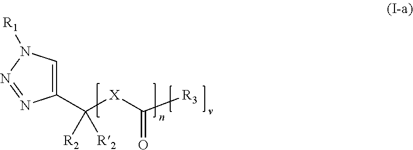

which forms a compound comprising chains depending on the units present in R1, such as units of ethylene oxide (CH₂—CH₂O).
The chain can also correspond to, for example but without being limited to these, (N-(2-hydroxypropyl)methacrylamide) (HMPA), PEG, Pluronic® (copolymer of ethylene oxide and propylene oxide), dextran (branched polysaccharide constituted by several glucose molecules), polyamides, etc.

When t=1, the polymer chain P is present in general formula (I) and there are therefore between 1 and 10 structures identical to or different from Formula (I) present on said polymer chain.

The term “spherical particle” denotes a structure that is constituted by several polymer chains P, in particular from 10¹² to 10¹⁶, in particular from 10¹⁴ to 10¹⁵ polymer chains P each comprising the molecule or molecules of Formula (I), identical or different, and which then form a spherical particle having an average diameter in the range from about 5 nm to about 100 μm depending on the units present in R1, such as units (CH₂—CH₂O: (EO)), and on the polymer chain P present in the molecule.

Preferably, the diameter of the particles is in the range from about 50 to 500 nm, in particular 300 nm for exploiting the tumour permeability effect.

On one particle, when t=1, there are therefore from 10¹² to 10¹⁷ structures identical to or different from Formula (I-a), preferably from 10¹⁵ to 10¹⁶.

Another advantage of the invention is the possibility of having particles of different sizes.

The expression “q is an integer in the range from 1 to 10” means that at least one molecule of Formula (I-a) is present on a polymer chain P and that the polymer chain P can comprise up to 10 molecules of Formula (I-a).

Said spherical particle can therefore be constituted by:
identical polymer chains P, each polymer chain comprising from 1 to 10 identical molecules of Formula (I-a), and it can be obtained by copolymerization of identical compounds of Formula (I-a) (R₁ representing a group of Formula (III)) with a mono- or polycyclic alkene, or
different polymer chains Pi, i varying from 1 to 10, for example polymer chains P comprising from 1 to 10 molecules of Formula (I-a) bearing an active ingredient, and polymer chains P2 comprising from 1 to 10 molecules of Formula (I-a) bearing a fluorophore, and it can be obtained by copolymerization of different compounds of Formula (I-a) (Rᵢ representing a group of Formula (III)) with a mono- or polycyclic alkene,
R′₁ chains comprising neither detecting probe, nor active ingredient, nor cell-penetrating peptide (CPP), nor triazole, which in particular serve to stabilize the particle.

The following Diagram A in the case when the monocyclic alkene is norbornene summarizes these various cases:

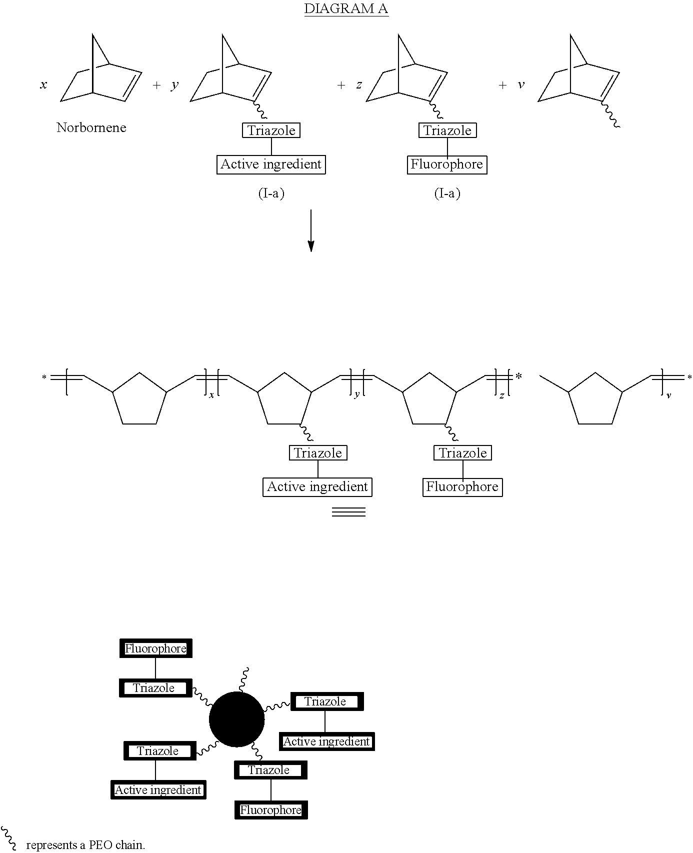

Throughout the description, nanovector or polymer nanovector also denotes:

a spherical particle constituted by polymer chains Pi comprising:
a compound of Formula (I) (t=1), or
a compound not comprising a polymer chain P but a molecule of Formula (I-a) in which R₁ corresponds to Formula (III), or
a molecule of Formula (I-a) in which R₁ corresponds to Formula (IV).

The structure of Formula “X(CO)” corresponds to a spacer E1 between R₃ and the carbon bearing R₂ and R′₂.

The expression “n represents 0 or 1” means that the spacer E1 formed by the structure of Formula “X(CO)” is or is not present in the compound of Formula (I).

When n=0, two cases are possible:
either v=1 and R₃ is then linked directly to the carbon bearing R₂ and R′₂, and Formula (I-a) therefore corresponds to the following general formula (I-b):

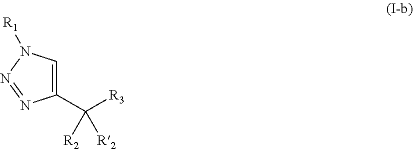

or v=0 and in this case R₂ and R′₂ form a ring as defined above, Formula (I-a) therefore corresponds to the following general formula (I-c):

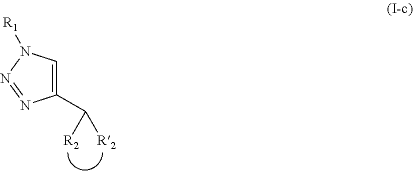

It is well understood that the compound of general formula (I) defined above can represent, when t=1 and q≧2, a particle or nanovector on which one or more molecules of Formula (I-a) and/or one or more molecules of Formula (I-b) and/or one or more molecules of Formula (I-c) are grafted.

When t=1, the R₁ group can correspond, for example, to one of the following Formulae II-a (when r=1) or II-b (when r=0):

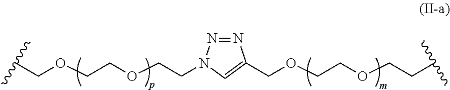

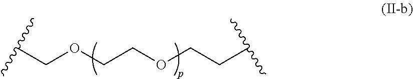

When t=0, the R₁ group can correspond, for example, to one of the following Formulae (III-a) (when r=1) or (III-b) (when r=0):

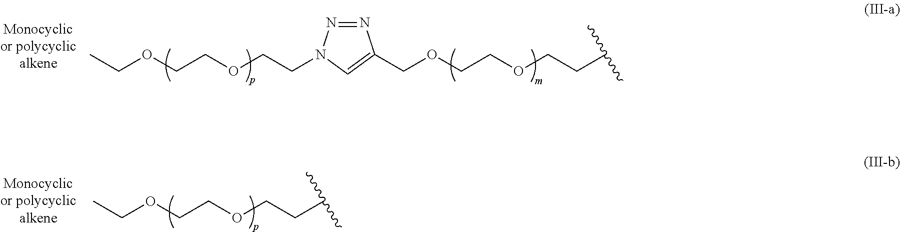

When t=0, R₁ can also correspond to a compound of the following Formula IV-a (when r=1) or (IV-b) (when r=0):

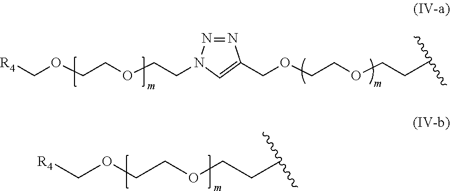

The term C₁-C₂₀ alkyl used in the definition of R′ and R″ and throughout the description denotes a linear or branched alkyl group comprising 1 to 20 carbon atoms.

By linear C₁ to C₂₀ alkyl group is meant: a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl and eicosyl group as well as all of their isomers.

By branched alkyl group is meant an alkyl group as defined above comprising substituents selected from the list of linear alkyl groups defined above, and said linear alkyl groups can also be branched.

C₃ to C₂₀ cycloalkyl group means a cyclopropyl, cyclobutyl, cyclopentyl, cyclohexyl, cycloheptyl, cyclooctyl, cyclononyl, cyclodecyl, cycloundecyl, cyclododecyl, cyclotridecyl, cyclotetradecyl, cyclopentadecyl, cyclohexadecyl, cycloheptadecyl, cyclooctadecyl, cyclononadecyl and cycloeicosyl group.

Such cycloalkyl groups can themselves be substituted by a linear or branched alkyl group as defined above.

The expression “active ingredient” denotes any pharmaceutical molecule that can have efficacy in any pathology whatever, in a mammal, in particular a human being, or a molecule detectable by any suitable method.

The expression “epigenetic modulator” denotes a molecule capable of reactivating regulator genes that have been repressed in mammalian tumour cells, in particular human tumour cells, such as for example the DNA methyl transferase inhibitors that inhibit the methylation of DNA and reactivate silent genes inducing differentiation, apoptosis or antiproliferation, or histone deacetylase inhibitors (HDACI or HDI) allowing the level of acetylation of histones to be increased and thus leading to re-expression of a silent gene.

The expression “detecting probe” denotes a molecule detectable by any suitable method, such as a fluorescent molecule (or fluorophore), for example fluorescein, rhodamine, or a radio-emitting molecule such as ⁹⁹Technetium, or contrast agents for medical imaging such as the lanthanides.

Said modulator or said probe is linked to the carbonyl of the spacer X(CO) of general formula (I) by an OH, NH₂ or SH function when n=1 or to the carbon bearing R₂ and R′₂ when n=0 and v=1.

The expression “cell-penetrating peptide” (CPP) denotes peptides, such as polyarginines and polylysines, but without being limited thereto, that can facilitate cell capture.

Said peptide is linked by its α-amino or carboxyl function or by the functions present on the side chain when they exist.

R₂ and R′₂ can be identical or different.

When R₂ and R′₂ are identical, the carbon carried by these two substituents is achiral.

By contrast, when R₂ and R′₂ are different, the carbon bearing the two substituents is chiral and the molecule of general formula (I) can be in racemic form, or in the form of each pure enantiomer (R) or (S) or of a mixture of the two enantiomers in the range from 0.01% (S)-99.9% (R) to 99.9% (S)-0.01% (R) provided that no other asymmetric carbon is present on the molecule of Formula (I).

If the molecule comprises one or more other asymmetric carbons, it can similarly be in racemic form, or of each pure enantiomer or of a mixture of the enantiomers and the molecule of general formula (I) then corresponds to a mixture of diastereoisomers.

When R₂ and R′₂ form a ring, R₂ and R′₂ represent, independently of one another, a phenyl, unsubstituted, or substituted by one or more substituents as defined above, and the two R₂ and R′₂ can then be identical or different, the ring being of the following general formula (V) or (V′):

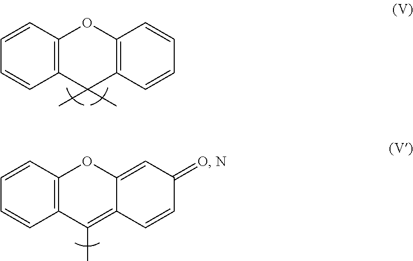

When the ring of Formula (V′) is substituted by a nitrogen, the substituents are as defined above for Y′ of Formula (Va) and the nitrogen is then in tetravalent N⁺ form and is associated with a monovalent anion as a counter-ion, such as for example a halide, HCO₃⁻, HSO₄⁻, PF₆⁻, CF₃SO₃⁻, CH₃COO⁻.

Preferably R₂ and R′₂ represent:
the ring of Formula (Va) above, and in particular the ring of Formula (Vaa) and in this case, n=0 and v=0, i.e. no R₃ group is present on the molecule of Formula (I) and the ring (Va) then forms the fluorophore, or
the ring of Formula (Vb), in particular the ring of Formula (Vbb), and in this case n=1 and the molecule of Formula (I) then has an R₃ group which represents an active ingredient, in particular an epigenetic modulator, or a cell-penetrating peptide, but not a detecting probe.

The compound of Formula (I), when q=1 and t=1, is therefore constituted by a triazole substituted:

on the one hand by R₁ which is itself bound to a polymer chain P, to a monocyclic or polycyclic alkene, or to a substituent R₄, and

on the other hand by a methylene or biphenyl methylene which is if appropriate bound to an active ingredient, in particular an epigenetic modulator, to a fluorescence probe or to a cell-penetrating peptide, optionally via a spacer XC(O).

It can also comprise R′1, which is then constituted solely by PEO.

The compounds of Formula (I) have a molecular weight in the range from 40 kDa to more than about 3200 kDa, in particular more than about 200 kDa

The inventors surprisingly found that the presence of the triazole, which is easily synthesized by bioconjugation (or “click” chemistry), not only made it possible to easily obtain the compounds of Formula (I) irrespective of which R₃ is group present on the latter, but still allowed the formation of a stable carbocation and therefore release of the active ingredient in an acid environment.

Another advantage of the invention is that the molecular weight of the compounds of Formula (I) and (Ia) allows them to circulate for a long time in the blood vessels of a mammal, in particular of a human being, and in particular for about 24 h, thus avoiding their elimination in particular in the case of the compounds >40 kDa and allows them to be trapped by enhanced permeability and retention (EPR) and then internalized in the cell by endocytosis and to be able to release the active ingredient or the detecting probe by the endosome/lysosome route in the reticuloendothelial system owing to the acid pH, by cleavage of the spacer E1, in particular for compounds larger than 200 kDa and in particular in the case of compounds larger than 3200 kDa, or by another route for the active ingredients that are sensitive to the acid environment, which can penetrate the nucleus by the nuclear pore complex (NPC) mechanism based on the nuclear localization signals (NLS).

Once the active ingredient has been released and/or the detecting probe has been released, the polymer chain P and the constituents other than the active ingredient or the detecting probe can be eliminated from the cell.

Yet another advantage of the invention is that as cleavage of the active ingredient only takes place at pH below 7, the compound of Formula (I) can circulate in the blood vessels without being degraded and therefore penetrates into the cell in its complete form.

Yet another advantage of the invention is that as the compounds of Formula (I) are designed starting from structures based on PEG, they will also be invisible to macrophages, thus evading elimination by said macrophages and thus allowing trapping by EPR.

In an advantageous embodiment, the polymer chain P comprises more than 10 molecules of Formula (I-a), identical or different, or constituting a mixture of one or more identical molecules with one or more different molecules as defined above.

In an advantageous embodiment, the invention relates to nanovectors of general formula (I) as defined above, in which the monomer units are derived from the polymerization of monocyclic alkenes, and are of the following Formula (Z1)

═[CH—R₅—CH]═ (Z1)
in which R₅ represents a hydrocarbon chain with 2 to 10 carbon atoms, saturated or unsaturated.
By “hydrocarbon chain” is meant a C₂ to C₁₀ alkyl chain.

In an advantageous embodiment, the invention relates to nanovectors of general formula (I) as defined above, in which the monocyclic alkenes from which the monomer units originated are:
cyclobutene, leading to a polymer comprising monomer units of the following Formula (Z1a):

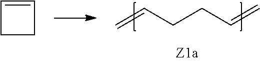

cyclopentene, leading to a polymer comprising monomer units of the following Formula (Z1b):

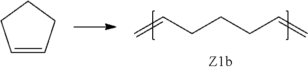

cyclopentadiene, leading to a polymer comprising monomer units of the following Formula (Z1c)

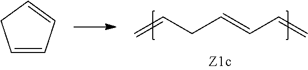

cyclohexene, leading to a polymer comprising monomer units of the following Formula (Z1d)

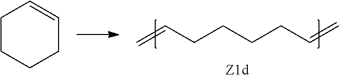

cyclohexadiene, leading to a polymer comprising monomer units of the following Formula (Z1e)

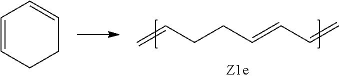

cycloheptene, leading to a polymer comprising monomer units of the following Formula (Z1f)

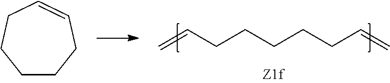

cyclooctene, leading to a polymer comprising monomer units of the following Formula (Z1h)

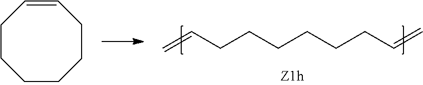

cyclooctapolyene, in particular cycloocta-1,5-diene, leading to a polymer comprising monomer units of the following Formula (Z1i)

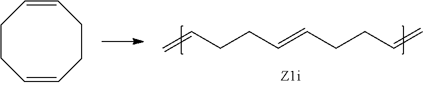

cyclononene, leading to a polymer comprising monomer units of the following Formula (Z1j)

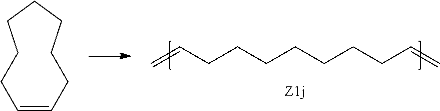

cyclononadiene, leading to a polymer comprising monomer units of the following Formula (Z1k)

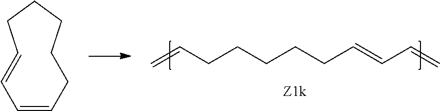

cyclodecene, leading to a polymer comprising monomer units of the following Formula (Z11)

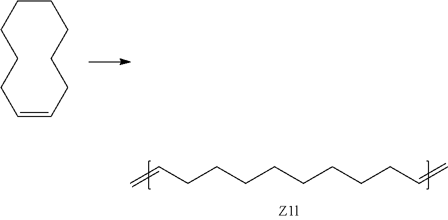

cyclodeca-1,5-diene, leading to a polymer comprising monomer units of the following Formula (Z1m)

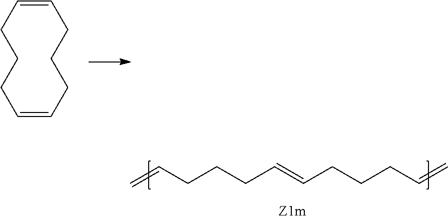

cyclododecene, leading to a polymer comprising monomer units of the following Formula (Z1n)

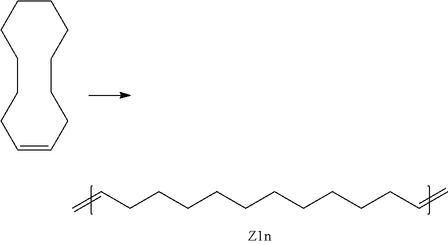

or also 2,3,4,5-tetrahydrooxepin-2-yl acetate, cyclopentadecene, paracyclophane, ferrocenophane.

In an advantageous embodiment, the invention relates to nanovectors of general formula (I) as defined above, in which the monomer units are derived from the polymerization of polycyclic alkenes, and are:
of the following Formula (Z2)

═[CH—R₆—CH]═ (Z2)
in which R₆ represents:
   * a ring of Formula

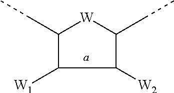

in which:
   W represents —CH₂—, or a heteroatom, or a —CHR₇-group, or a group —CHR₈—, R₇ representing a chain comprising a poly(ethylene oxide) of Formula —(CH₂—CH₂—O)ₘ, m being as defined above and R₈ representing a C₁ to C₁₀ alkyl or alkoxy chain,
   W₁ and W₂, independently of one another, represent H, or an R₇ chain, or an R₈ group mentioned above, or form, in combination with the carbon atoms bearing them, a ring of 4 to 8 carbon atoms, this ring being if appropriate substituted by an R₇ chain or an R₈ group mentioned above,
   “a” represents a single or double bond,
* or a ring of Formula

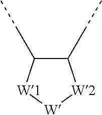

in which:
   W′ represents —CH₂—, or a heteroatom, or a —CHR₇— group, or a —CHR₈— group, R₇ and R₈ being as defined above,
   W′ᵢ and W′₂, independently of one another, represent —CH₂—, or a —C(O) group, or a —COR₇ group, or a —C—OR₈ group, R₇ and R₈ being as defined above,
of the following Formula (Z3)

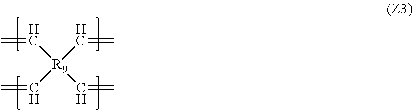

in which R₉ represents:
   * a ring of Formula

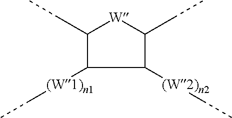

in which:
   n₁ and n₂, independently of one another, represent 0 or 1,
   W″ represents —CH₂—, or a —CHR₇— group, or a —CHR₈— group, R₇ and R₈ being as defined above,
   W″ᵢ and W″₂, independently of one another, represent a hydrocarbon chain with 0 to 10 carbon atoms,
   * or a ring of Formula

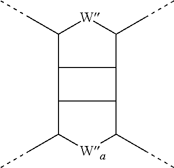

in which W″ and W″ₐ, independently of one another, represent —CH₂—, or a —CHR₇-group, or a —CHR₈— group, R₇ and R₈ being as defined above,
* or a ring of Formula

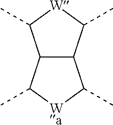

in which W″ and W″ₐ, independently of one another, represent —CH₂—, or a —CHR— group, or a —CHR₈— group, R₇ and R₈ being as defined above.

In an advantageous embodiment, the invention relates to nanovectors of general formula (I) as defined above, in which the polycyclic alkenes from which the monomer units originate are:
the monomers containing a cyclobutene ring, leading to a polymer comprising monomer units of the following Formula (Z2a):

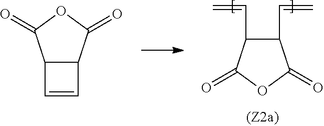

the monomers containing a cyclopentene ring, leading to a polymer comprising monomer units of the following Formula (Z2b):

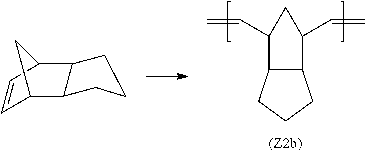

norbornene (bicyclo[2,2,1]hept-2-ene), leading to a polymer comprising monomer units of the following Formula (Z2c):

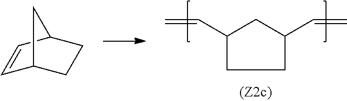

norbornadiene, leading to a polymer comprising monomer units of the following Formula (Z2d):

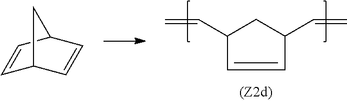

7-oxanorbornene, leading to a polymer comprising monomer units of the following Formula (Z2e):

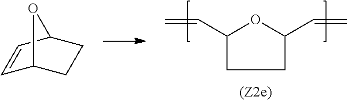

7-oxanorbornadiene, leading to a polymer comprising monomer units of the following Formula (Z2f):

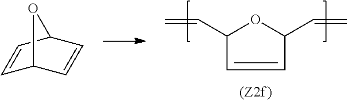

the norbornadiene dimer, leading to a polymer comprising monomer units of the following Formula (Z3a):

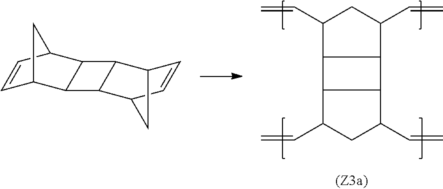

dicyclopentadiene, leading to a polymer comprising monomer units of the following Formula (Z3b):

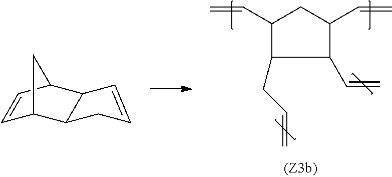

tetracyclododecadiene, leading to a polymer comprising monomer units of the following Formula (Z3c):

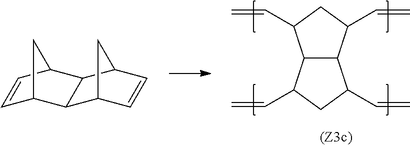

or bicyclo[5,1,0]oct-2-ene, bicyclo[6,1,0]non-4-ene.

In an advantageous embodiment, the invention relates to nanovectors of general formula (I) as defined above, in which the mono- or polycyclic alkenes from which the monomer units originate are:
norbornene (bicyclo[2,2,1]hept-2-ene), leading to a polymer comprising monomer units of Formula (Z2c),
tetracyclododecadiene, leading to a polymer comprising monomer units of Formula (Z3c),
dicyclopentadiene, leading to a polymer comprising monomer units of Formula (Z3b),
the norbornadiene dimer, leading to a polymer comprising monomer units of Formula (Z3a),
cycloocta-1,5-diene, leading to a polymer comprising monomer units of Formula (Z1i).

In an advantageous embodiment, the invention relates to a compound of general formula (I) as defined above, in which the epigenetic modulator is selected from:
a nucleoside, in particular cytidine, uridine, adenosine, guanosine, thymidine or inosine,
histone deacetylase inhibitors (HDI), in particular Zolinza® (SAHA), trichostatin A (TSA), valproic acid, MS-275 or CI-994, or
DNA methyltransferase inhibitors (DNMTI), in particular 5-azacytidine, 5-aza-2′-deoxycytidine and zebularine.

The nucleosides are glycosylamines constituted by a nucleobase (or base) bound to a ribose or a deoxyribose via a glycosidic bond or a base bound to an analogue of ribose such as in gemcitabine.

The biological activity of the histone deacetylase inhibitors (HDI or HDAC inhibitors) leads to an increase in the level of acetylation of the histones, which allows re-expression of silent tumour regulator genes in the tumour cells.

Zolinza® (SAHA) has the following structure:

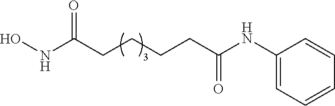

SAHA, when present, is linked either to the spacer X(CO) or to the carbon bearing R₂ and R′₂ of Formula (I) by the hydroxyl of the hydroxamic acid function.

Trichostatin (TSA) has the following structure:

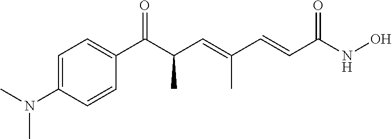 [SMILES: CC(/C=C/C(=O)NO)=C\[C@@H](C)C(=O)c1ccc(N(C)C)cc1]

TSA, when present, is linked either to the spacer X(CO) or to the carbon bearing R₂ and R′₂ of Formula (I) by the hydroxyl of the hydroxamic acid function or by the ketone function close to the aromatic ring.

Valproic acid has the following structure:

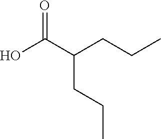

Valproic acid, when present, is linked either to the spacer X(CO), or to the carbon bearing R₂ and R′₂ of Formula (I) by the hydroxyl of the acid function.

MS-275 has the following structure:

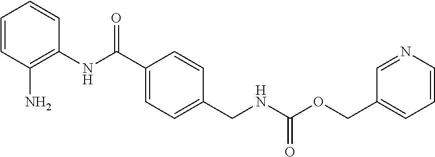 [SMILES: Nc1ccccc1NC(=O)c1ccc(CNC(=O)OCc2cccnc2)cc1]

MS-275, when present, is linked either to the spacer X(CO) or to the carbon bearing R₂ and R′₂ of Formula (I) by the amine function of the aniline moiety.

CI-994 has the following structure:

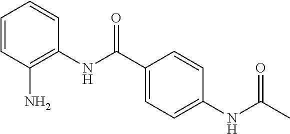 [SMILES: CC(=O)Nc1ccc(C(=O)Nc2ccccc2N)cc1]

CI-994, when present, is linked either to the spacer X(CO) or to the carbon bearing R₂ and R′₂ of Formula (I) by the amine function of the aniline moiety.

Hypermethylation of the DNA regions called CpG islets is responsible for the poor activation of the promoter genes involved in the regulation of the transcription. The action of the DNA methyltransferase inhibitors (DNMT inhibitors) results in blocking of this abnormal methylation.

5-Azacytidine has the following structure:

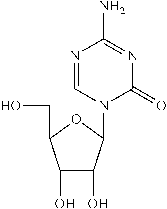 [SMILES: Nc1ncn(C2OC(CO)C(O)C2O)c(=O)n1]

5-Azacytidine is bound to the spacer X(CO) or to the carbon bearing R₂ and R′₂ of Formula (I) by its primary or secondary alcohol function or by the amine.

5-Aza-2′-deoxycytidine (or decitabine) has the following structure:

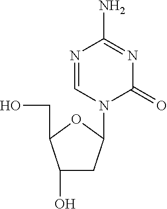 [SMILES: Nc1ncn(C2CC(O)C(CO)O2)c(=O)n1]

5-Aza-2′-deoxycytidine is bound to the spacer X(CO) or to the carbon bearing R₂ and R′₂ of Formula (I) by its primary or secondary alcohol or amine function.

Zebularine has the following structure:

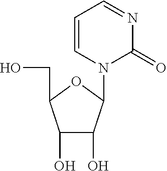 [SMILES: O=c1ncccn1C1OC(CO)C(O)C1O]

Zebularine is bound to the spacer X(CO) or to the carbon bearing R₂ and R′₂ of Formula (I) by its primary or secondary alcohol function.

In an advantageous embodiment, the nanovector comprises an epigenetic modulator which is a histone deacetylase inhibitor: SAHA and corresponds to the compound product denoted (20e).

In an advantageous embodiment, the invention relates to nanovectors of general Formula (I) as defined above, in which the detecting probe is selected from a fluorophore, in particular rhodamine B or fluorescein, coumarins, in particular 7-hydroxy-4-methylcoumarin, the Bodipy dyes, Texas red, the cyanines, especially the CY3 or CY5 dyes, or a radio-emitting substance such as ⁹⁹Technetium in liganded form, or contrast agents for medical imaging such as the lanthanides (gadolinium).

The expression “fluorophore” denotes a chemical substance capable of emitting fluorescent light after excitation.

Rhodamine has a skeleton with the following structure:

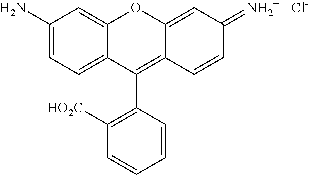 [SMILES: [Cl-] ; Nc1ccc2c(-c3ccccc3C(=O)O)c3ccc(=[NH2+])cc-3oc2c1]

The CO₂H function then corresponds to the spacer E1.

Rhodamine B has the following structure:

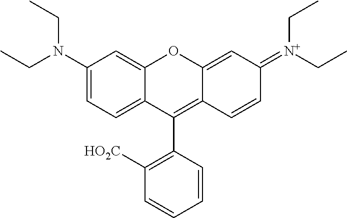 [SMILES: CCN(CC)c1ccc2c(-c3ccccc3C(=O)O)c3ccc(=[N+](CC)CC)cc-3oc2c1]

The CO₂H function then corresponds to the spacer E1.

Fluorescein has the following structure:

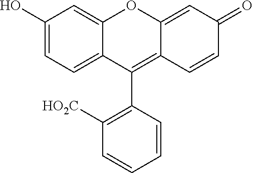 [SMILES: O=C(O)c1ccccc1-c1c2ccc(=O)cc-2oc2cc(O)ccc12]

The CO₂H function then corresponds to the spacer E1.

7-Hydroxy-4-methylcoumarin has the following structure:

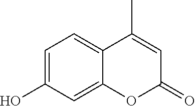

The OH function is then linked to the carbon bearing R₂ and R′₂ of Formula (I) without a spacer.

The Bodipy dyes correspond to the abbreviation of boron-dipyromethene, and represent a family of dyes that absorb strongly in the UV and have the property of emitting narrow fluorescence with a high quantum yield. They are all derived from 4,4-difluoro-4-bora-3a,4a-diaza-s-indacene shown below:

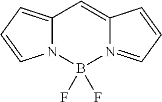

Texas red has the following Formula:

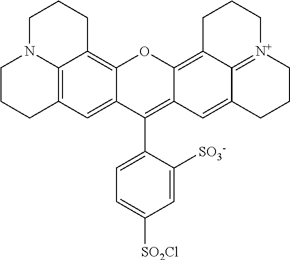

The CY3 dyes have the following Formula:

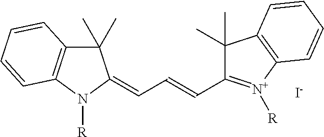

R representing an alkyl, such as methyl, ethyl etc.

The CY5 dyes have the following Formula:

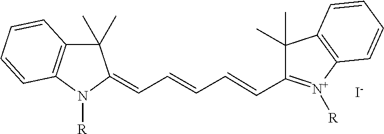

R representing an alkyl, such as methyl, ethyl etc.

The CY7 dyes have the following Formula:

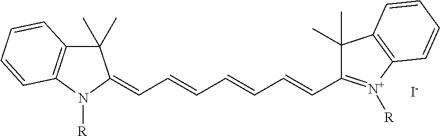

R representing an alkyl, such as methyl, ethyl etc (Synthesis and In Vivo Fate of Zwitterionic Near-Infrared Fluorophores. Hak Soo Choi, Khaled Nasr, Sergey Alyabyev, Dina Feith, Jeong Heon Lee, Soon Hee Kim, Yoshitomo Ashitate, Hoon Hyun, Gabor Patonay, Lucjan Strekowski, Maged Henary, John V. Frangioni. Angew. Chem. Int. Ed. 2011, 50, 6258-6263)

⁹⁹Technetium in liganded form can correspond to technetium gluceptate or more conventionally complexes of the diethylene triamine pentaacetate type (DTPA) (Brain Research Protocols 8 (2001) 143-149, Non-invasive assessment of blood-brain barrier (BBB) permeability 99 using a gamma camera to detect technetium-gluceptate extravasation in rat brain. Pamela Esposito, Stanley Jacobson, Raymond Connolly, Daniela Gheorghe, Theoharis C. Theoharides; Use of sequential dtpa clearance and high resolution computerized tomography in monitoring interstitial lung disease in dermatomyositis. C. Hell, E. Romas, B. Kikham. British Journal of Rheumatology 1996; 35: 164-166)

A contrast agent is a compound that artificially increases contrast, making it possible to visualize an anatomical structure (for example, an organ) or pathological structure (for example, a tumour) having little or no natural contrast.

The principle of operation of the contrast product depends on the imaging technique used: in radiography, the product”s ability to absorb X-rays is exploited; in magnetic resonance imaging, the compounds used are selected depending on their magnetic properties; in ultrasonography, substances having a characteristic echo to ultrasound are used.

In an advantageous embodiment, the invention relates to nanovectors of general formula (I) as defined above, in which the cell-penetrating peptide is selected from polylysines, polyarginines, polylysines modified by imidazoles, or mimetics of polyglycines with a chain bearing a nitrogen-containing end group.

In both cases, the COOH or NH₂ termination can be modified for bearing an alkyne for click chemistry.

The various compounds mentioned are shown below:

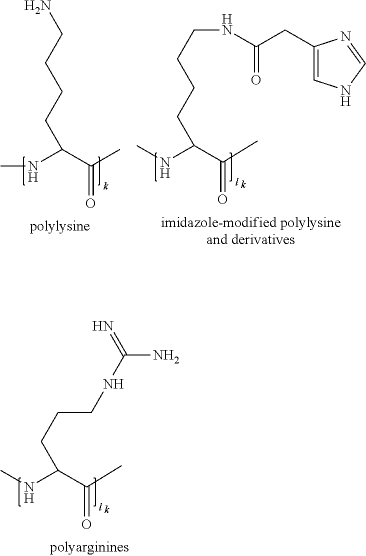

in which k is in the range from 1 to 10.

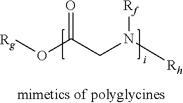

in which i varies from 2 to 10 and R_{f} represent a C₁-C₂₀ alkyl bearing a nitrogen-containing group (NR^{a}R^{b}) or a C₃-C₂₀ cycloalkyl bearing a nitrogen-containing group (NRₐR_{b}), with a and b representing a C₁-C₂₀ alkyl or a C₃-C₂₀ cycloalkyl. (NRₐR_{b}) can also be in the form of ammonium (NRₐR_{b}R_{c}) with a, b and c defined as above. R_{g} and Rₕ represent an alkyne, or a hydrogen or a C₁-C₂₀ alkyl or a C₃-C₂₀ cycloalkyl. R_{f} can optionally represent an alkyne among the i repetitions.

The presence of a cell-penetrating peptide (CPP) can facilitate capture of the compound of Formula (I) by a cell. For molecules that are weak bases with possible accumulation in the lysosome or that do not support the hydrolysis activity of the lysosome (such as nucleotides, peptides, DNA), the bypassing of the endocytosis route at the level of the endosome is very substantial. The nitrogen-rich cationic polymers such as the polyarginines, polylysines or polylysines modified with imidazoles (imidazole-modified polylysines) make it possible to destabilize the endosomes by altering the pH, leading to rupture of the endosome membrane.

Consequently, the nanovectors bearing CPP also carry one or more active ingredient(s) and optionally one or more detecting probe(s).

According to another aspect, the present invention relates to compounds of general formula (III) as precursor of the polymer chain P of Formula (I).

According to another aspect, the present invention relates to nanovectors as defined above, for use as medicament and/or diagnostic agent.

The spherical particles, when they have previously been administered to a mammal and in particular a human being, and in which q=1 and R₃ represents an active ingredient, are used as medicament after release of the active ingredient in the cell after internalization in the cell by endocytosis.

When R₃ represents a detecting probe, the spherical particles, when they have previously been administered to a mammal and in particular a human being, can be used as diagnostic agent after release of the probe in the cell after selective trapping of the compound by EPR and internalization by endocytosis by a cell, in particular a tumour cell, making it possible to diagnose and/or locate a pathology.

This same detecting probe also makes it possible to monitor cell trafficking.

The spherical particles, when they have previously been administered to a mammal and in particular a human being, and in which various polymer chains are present, and comprising for example at least one active ingredient and at least one detecting probe, are used both as medicament and as agent for monitoring internalization of the active ingredient in the target cell, in particular a cancer cell, after release of the active ingredient and of the detecting probe in the cell after internalization of the spherical particles in the cell by endocytosis.

In an advantageous embodiment, the nanovectors for use as medicament and/or diagnostic agent are nanovectors in which R₁ represents a group of Formula (III).

Yet another advantage of the invention is that the compound of Formula (I) whether it is in the form of polymer (t=1) or in the form of monocyclic or polycyclic alkene (t=0) can still be trapped by EPR in the cell and can then undergo endocytosis and thus be used as medicament and/or diagnostic agent.

In an advantageous embodiment, the nanovectors for use as medicament and/or diagnostic agent are nanovectors in which R₁ represents a group of Formula (IV).

Yet another advantage of the invention is that the nanovectors can also be without polymer (t=0) or monocyclic or polycyclic alkene (t=0 and R1 is of Formula IV) while allowing endocytosis and also being used as medicament and/or diagnostic agent.

In an advantageous embodiment, the present invention relates to nanovectors as defined above, for use as medicament and/or diagnostic agent, in which the active ingredient, such as an epigenetic modulator, and/or the detecting probe are released in the cell after endocytosis by said cell at an acid pH.

After penetration into the cell by endocytosis, the compound of general formula (I) is internalized in the endosome in which the pH is 6, allowing commencement of the hydrolysis of the group or groups R₃ present and the maturation of which leads to the lysosome in which pH is 5, which can lead to complete hydrolysis of the group or groups R₃ and release of the active ingredient or active ingredients in the cytoplasm (FIG. 1).

In an advantageous embodiment, the present invention relates to nanovectors as defined above, for use as medicament and/or diagnostic agent, in particular for treating and/or diagnosing disorders selected from neurological diseases, inflammatory processes, cancer, diseases of the blood, etc.

Bt the expression “neurological diseases” is meant, without being limited to, Alzheimer”s disease, Parkinson”s disease, multiple sclerosis, neuropathy, polyneuritis, epilepsy, meningitis, etc.

The expression “inflammatory process” denotes, without being limited to, arthritis, arteritis, colitis, conjunctivitis, cystitis, dermatitis, encephalitis, endocarditis, endometritis, gastritis, meningitis, myocarditis, myelitis, pancreatitis, peritonitis, sinusitis, tendinitis.

The expression “cancer” denotes, without being limited to, haematopoietic cancers, leukaemias, lymphomas, carcinomas, adenocarcinomas, sarcomas, melanoma, head and neck carcinoma, cancer of the oesophagus, buccal cancer and cancer of the pharynx, cancer of the larynx, bladder cancer, colorectal cancer, ovarian cancer, uterine cancer, cancer of the penis, cancer of the vulva and vagina, cervical cancer, prostate cancer, renal cancer, skin cancer, bone cancer, cancer of the joints and joint cartilages, testicular cancer, stomach cancer, gastrointestinal cancer, genito-urinary cancer, lung cancer, thymoma, mesothelioma, teratoma, brain cancer, liver cancer, pancreatic cancer, glioma, glioblastoma, oligoastrocytoma, meningioma, hypophyseal adenoma, glioblastoma multiforme, medulloblastoma, ependymoma, anaplastic astrocytoma, oligodendroglioma, thyroid cancer, anaplastic thyroid cancer, haemangiosarcoma, Kaposi sarcoma, lymphangiosarcoma, ganglionic and extraganglionic malignant lymphomas, Hodgkin's lymphoma, indolent non-Hodgkin's lymphomas, retinoblastoma.

The expression “diseases of the blood” relates to diseases that affect the erythrocytes, leukocytes, and platelets, and denotes, without being limited thereto:
haemoglobinopathies, in particular thalassaemias, drepanocytosis, haemoglobin C, methaemoglobinaemia,
enzyme deficiencies, such as glucose-6-phosphate dehydrogenase (G₆PD or G₆PDH) deficiency, pyruvate kinase deficiency,
lowering of cell counts, such as aplasia, anaemias, leukopenias, thrombocytopenias,
increase in cell counts, such as leukocytosis, thrombocytosis,
malignant haemopathies such as lymphomas, myelomas, leukaemia, erythropoiesis,
coagulopathies such as platelet abnormalities, primary haemostasis, abnormalities of proteins, thrombotic abnormalities.

In an advantageous embodiment, the present invention relates to nanovectors as defined above, for use as medicament and/or diagnostic agent, in particular for combination treatment of pathologies selected from neurological diseases, inflammatory processes, cancer, and diseases of the blood.

By “combination treatment” is meant both particles bearing at least two active ingredients for treating different disorders and particles bearing at least two different active ingredients for treating the same pathology.

For example, the same particles can comprise an HDI and a DNMT inhibitor for treating malignant pleural mesothelioma (MPM).

The HDIs and DNMTs are anticancer drugs that are currently being tested in clinical trials alone or in combination in the treatment of MPM.

However, severe side-effects can be observed with these treatments mainly because of the non-selective action of these molecules.

Consequently, an advantage of the particles of the invention comprising at least two different active ingredients, in particular an HDI and a DNMT inhibitor, is being able to selectively target the cell, in particular the cancer cell.

According to another aspect, the invention relates to a pharmaceutical composition comprising nanovectors as defined above as active ingredient, in combination with a pharmaceutically acceptable vehicle.

By “pharmaceutical composition” is meant a composition comprising one or more active ingredient(s) constituted by nanovectors, which can be administered to a patient for treating a pathology as defined above.

By “pharmaceutically acceptable vehicle” is meant any substance other than the active ingredient in a medicament. Addition thereof is intended to endow the final product with physicochemical and/or biochemical characteristics for promoting administration, while preferably avoiding covalent chemical interactions with the active ingredients.

In an advantageous embodiment, the pharmaceutical composition as defined above is in a form that can be administered by intravenous route at a unit dose of 5 mg to 500 mg.

The compositions for administration by intravenous route can be sterile solutions or emulsions. As solvent or vehicle, it is possible to use water, propylene glycol, a polyethylene glycol, vegetable oils, in particular olive oil, injectable organic esters, for example ethyl oleate. These compositions can also contain adjuvants, in particular wetting agents, isotonic agents, emulsifiers, dispersants and stabilizers.

Administration at the above unit dose can be carried out once in 24h or can be repeated depending on the pathology and the medical prescription.

In an advantageous embodiment, the pharmaceutical composition as defined above is in a form that can be administered by intravenous route at a dose in the range from about 0.05 μg/kg to about 10 mg/kg.

According to another aspect, the invention relates to nanovectors constituted by polymer chains of general formula (I) as defined above, comprising a step of ring-opening metathesis polymerization and a step of bioconjugation.

There are two possible approaches to synthesis of the nanovectors of the invention:
either first carry out a step of ring-opening metathesis polymerization (ROMP) and then a key step of bioconjugation (click chemistry),
or first carry out a key step of bioconjugation (click chemistry) and then a step of ring-opening metathesis polymerization (ROMP) (FIG. 2).

The ROMP step, well known to a person skilled in the art, requires bringing a catalyst such as:
Bis(tricyclohexylphosphine)benzylidine ruthenium(IV) dichloride (PCy₃)₂Cl₂Ru=CHPh): first-generation Grubbs complex (Grubbs et al., J. Am. Chem. Soc, 1996, 118, 100-110),
tricyclohexylphosphine[1,3-bis(2,4,6-trimethylphenyl)-4,5-dihydro-imidazol-2-ylidene][benzylidene]ruthenium(IV) dichloride (H2Imes)(PCy₃)Cl₂Ru=CHPh): second-generation Grubbs complex (Grubbs et al., Organic Letters, 1999, 1(6), 953-956),
or catalysts such as Schrok (J. J. Murphy, T. Kawasaki, M. Fujiki, K. Nomura. Macromolecules 2005, 38, 1075-83) or based on other metals (a) S. Hayano, Y. Takeyama, Y. Tsunogae, I. Igarashi Macromolecules, 2006, 39, 4663-70. B) Y. Zou, D. Wang, K. Wurst, C. Kühnel, I. Reinhardt, U. Decker, V. Gurram, S. Camadanli, M. R. Buchmeiser. Chem. Eur. J. 2011, 17, 13832-13846),
   into contact with a mono- or polycyclic alkene (Chemtob A., Gnanou Y., Heroguez V., Macromolecules, 2002, 35(25), 9262-9269) to form the polymer.

The step of bioconjugation (or click chemistry) requires bringing an alkyne into contact with a nitride in the presence of Cu(I) to obtain the corresponding triazole.

Yet another advantage of the invention is to provide a method involving a key step of bioconjugation between two compounds, one bearing a nitride function and the other an alkyne function that are easily accessible, carried out under mild conditions allowing easy access to particles comprising active ingredients and/or detecting probes that are various and/or sensitive and have other functionalities.

In an advantageous embodiment, the present invention relates to a method for preparing nanovectors constituted by polymer chains of general formula (I) in which R₁ is a group of general formula (II) as defined above, characterized in that the step of ring-opening metathesis polymerization is carried out prior to the step of bioconjugation.

In an advantageous embodiment, the present invention relates to a method for preparing nanovectors constituted by polymer chains of general formula (I) in which R₁ is a group of general formula (II) as defined above, in which the step of ring-opening metathesis polymerization is carried out prior to the step of bioconjugation, as defined above, comprising the following steps:
a. Preparation of a compound of the following general formula (VI-a) comprising a monocyclic or polycyclic alkene and a nitride function:

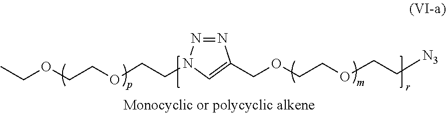

p and r being as defined above,
b. Implementation of the step of ring-opening metathesis polymerization in the presence of a catalyst to form a compound of Formula (VII) comprising nitride functions on the surface of a polymer:

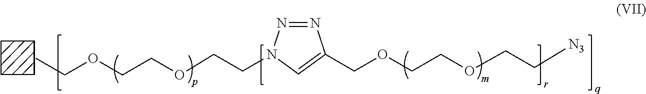

m, r, p and q being as defined above,
c. Preparation of a compound of general formula (VIII) comprising an alkyne function:

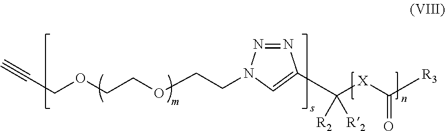

in which n, m, R₂, R′₂ and R₃ are as defined above and s represents 0 or 1, s is an integer in the range from 0 to 10, in particular 0 or 1,
d. Implementation of the bioconjugation step by bringing said compound of Formula (VII) into contact with the compound of Formula (VIII) in the presence of copper to obtain nanovectors constituted by a polymer chain of Formula (I) in which R₁ is a group of Formula (II), and R′1 is or is not present.

The compound of Formula VI-a can be prepared by techniques that are familiar to a person skilled in the art.

As a general rule, in step a., the mono- or polycyclic alkene methanol is reacted with ethylene oxide in the presence of a base, in particular diphenylmethyl potassium (Héroguez V, Breunig S, Gnanou Y, Fontanille M, Macromolecules 1996, 29, 4459) in an organic solvent such as THF, at ambient temperature to form a derivative of poly(ethylene)oxide containing an alkene.

After functionalization of the primary alcohol function free from the poly(ethylene)oxide comprising an alkene function, for example by a paratoluene sulphonyl group or a methane sulphonyl group, reaction with sodium nitride leads to compound (VI).

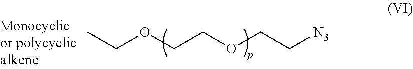

The compound of Formula (VI-a) is then prepared by a reaction of bioconjugation of compound (VI) with a poly(ethylene)oxide comprising an alkyne function at one end and an alcohol function at the other end by click chemistry, in a solvent mixture such as dichloromethane-water in the presence of copper I, in particular CuBr, then functionalization of the primary alcohol function that is still free. Reaction with sodium nitride then leads to compound (VI-a) in which r=1.

The repetition of this last step makes it possible to obtain the compounds (VI-a) in which r is in the range from 2 to 10.

In step b., the ROMP reaction is implemented, the compound (VI-a) comprising the mono- or polycyclic alkene being reacted in the presence of a catalyst as defined above, in particular the first-generation Grubbs catalyst, in a solvent such as a dichloromethane/ethanol mixture, at ambient temperature and stopping the reaction by adding a solvent such as vinylethyl ether to produce the compound (VII).

If the compound (VI-a) only comprises identical polymer chains P, then the particles formed only comprise a single type of polymer chains.

If compound (VI-a) comprises different polymer chains (P, P2, P3 etc.), the particles formed comprise several different polymer chains (P, P2, P3 etc.) but the total number of polymer chains on the particles remains unchanged.

In step c., the derivative (VIII) bearing the alkyne function that is the precursor of the triazole that will be formed by the key step of bioconjugation is obtained by reaction of R₂(CO)R′₂ (R₂ and R′₂ being as defined above, and the synthesis of which is well known to a person skilled in the art) with a trimethylethynylsilane in the presence of a base, such as butyllithium to produce the corresponding alcohol derivative:

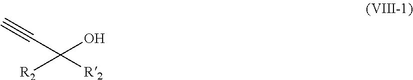

which by reaction of bioconjugation with for example a HO—CH₂CH₂—O—(CH₂CH₂O)ₚ—CH₂CH₂N₃ group makes it possible to obtain a group of Formula (XI-1):

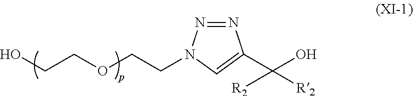

The group of Formula (XI-1) is then reacted with a base, for example NaH and a propargyl halide, for example propargyl bromide to form the compound of general formula (XI-2):

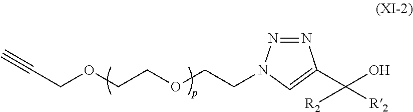

The compound of Formula (XI-2) is then reacted for example with a paranitrophenyl carbonate or carbonyl diimidazole and substituted by an alcohol, an amine, an acid etc., and makes it possible to obtain the compounds of general formula (VIII).

Step d. involves the key reaction of bioconjugation between the alkyne (VIII) and the nitride (VII) in the presence of copper as described for step a. above to produce the compounds of general formula (I) in which R₁ is a group of Formula (II).

In an advantageous embodiment, the present invention relates to a method for preparing nanovectors constituted by polymer chains of general formula (I) in which R₁ is a group of general formula (II) and t=1, or of general formula (III) and t=0, as defined above, in which the step of bioconjugation is carried out prior to the optional step of ring-opening metathesis polymerization.

In an advantageous embodiment, the present invention relates to a method for preparing nanovectors in which the step of bioconjugation is carried out prior to the optional step of ring-opening metathesis polymerization as defined above, comprising the following steps:
a. Preparation of a compound of the following general formula (VI-a) comprising a monocyclic or polycyclic alkene and a nitride function:

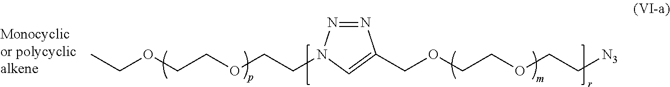

m, r and p being as defined above,
b. Preparation of a compound of general formula (VIII) comprising an alkyne function:

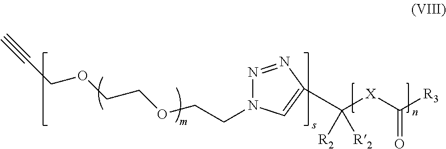

in which n and m are as defined above and s is an integer in the range from 0 to 10,
R₂, R′₂ and R₃ are as defined above.
c. Implementation of the bioconjugation step by bringing said compound of Formula (VI-a) into contact with the compound of Formula (VIII) in the presence of copper to obtain the compounds of general formula (I) in which t=0 and R₁ represents a group of Formula (III).
d. Optionally, implementation of the step of ring-opening metathesis polymerization in the presence of a catalyst to form nanovectors constituted by polymer chains of general formula (I) in which R₁ is a group of general formula (II) and t=1.

In this embodiment, the implementation of the ROMP polymerization step leads to the compound of general formula (I) in which R₁ is a group of general formula (II). If the ROMP polymerization step is not implemented, the compound of general formula (III) with t=0 is then obtained.

The compound of Formula VI-a is prepared by the method described above, some of which are given in the examples section.

The compounds of Formula VIII can be prepared as indicated above.

The compounds of Formula VIII in which s=0 (VIII-a) bearing the alkyne function that is the precursor of the triazole formed by the bioconjugation step are obtained by reaction of R₂(CO)R′₂ with a trimethylethynylsilane in the presence of a base, such as butyllithium to produce the corresponding alcohol derivative (VIII-b):

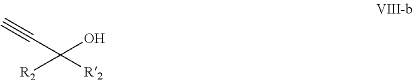

which by reaction with for example a paranitrophenyl carbonate or carbonyl diimidazole and substitution with an alcohol, an amine, an acid etc., makes it possible to obtain the compounds of general formula (VIII-a):

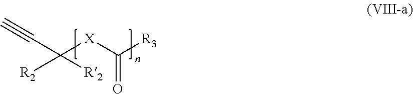

The invention is illustrated by the drawings and the examples given below.

### DESCRIPTION OF THE DRAWINGS

FIG. 1 shows the monitoring of a nanovector (colloid, white sphere) of the invention comprising a CPP (white square), a fluorescent detecting probe (grey square) and an active ingredient (black square), from its circulation in the blood vessels until release of the active ingredient in the cytoplasm.

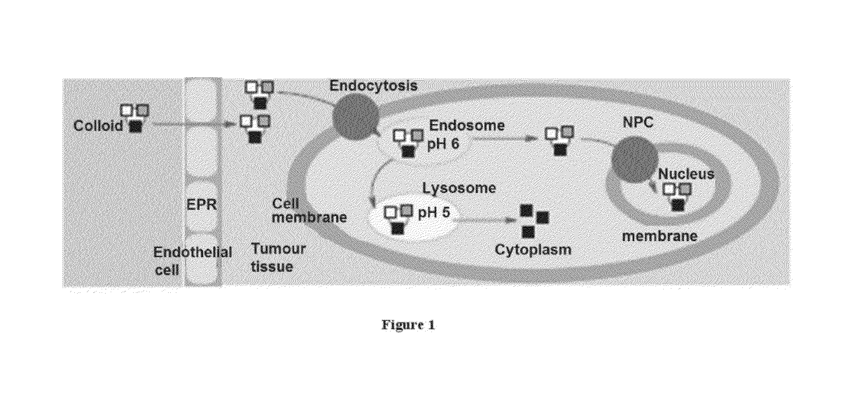

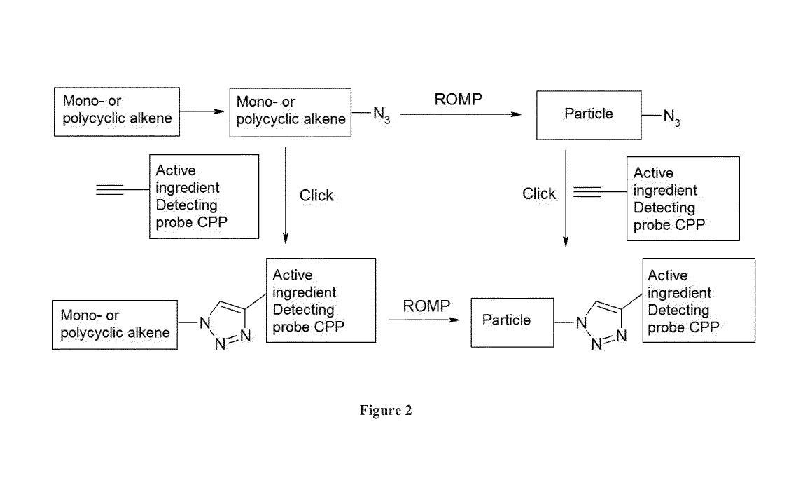

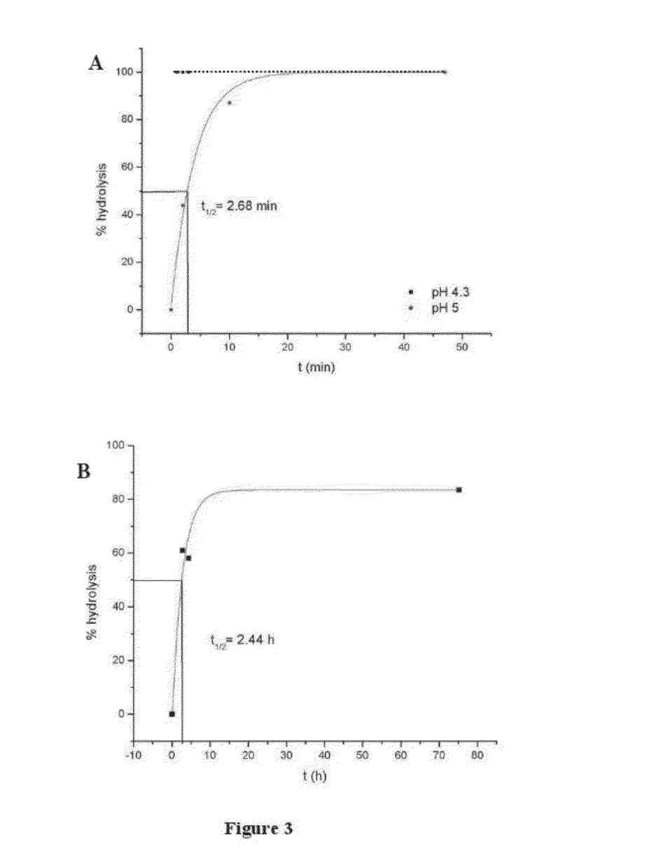

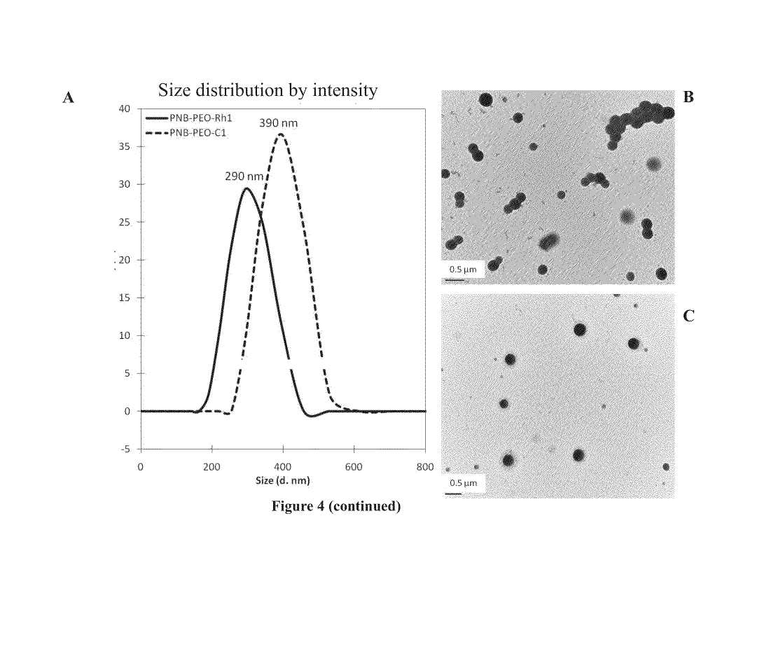

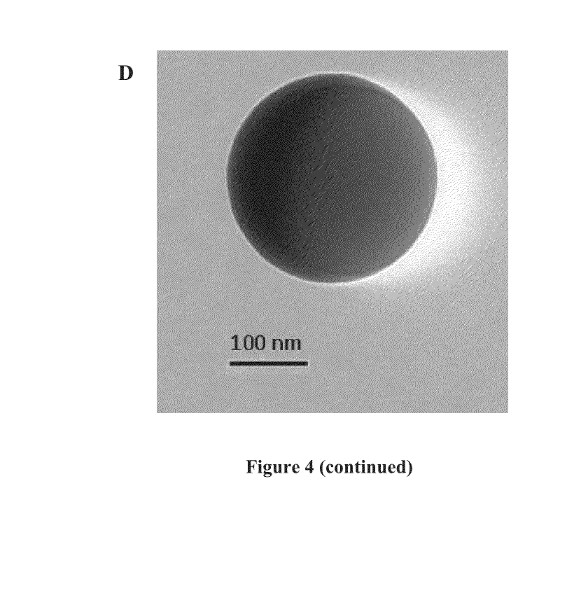

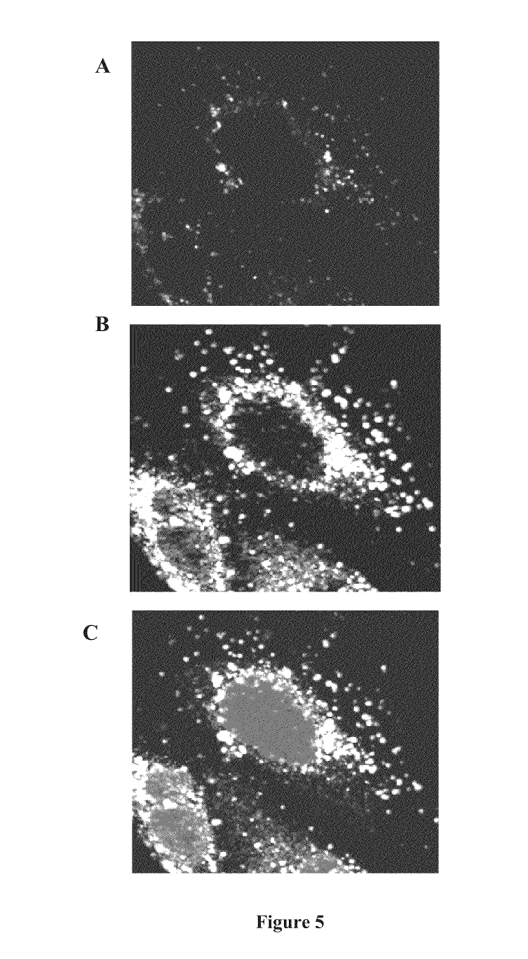

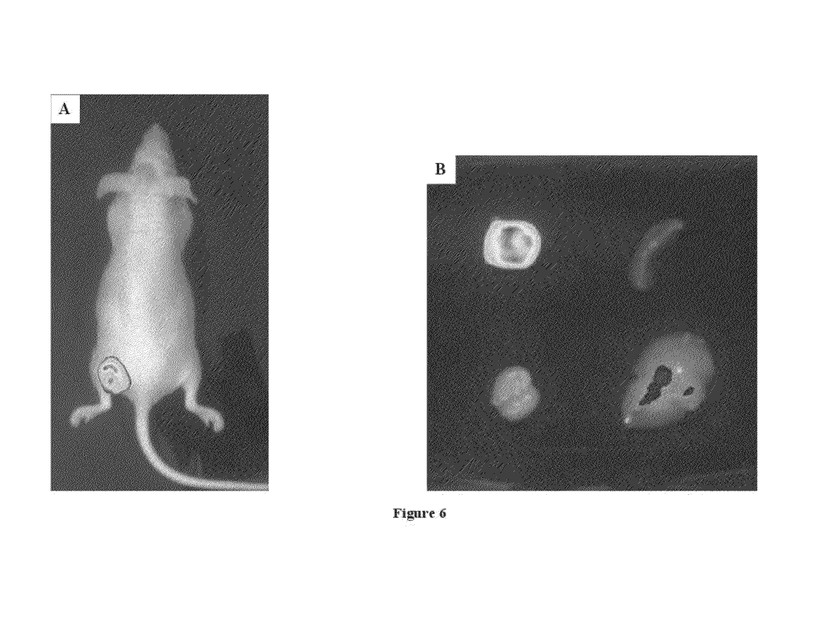

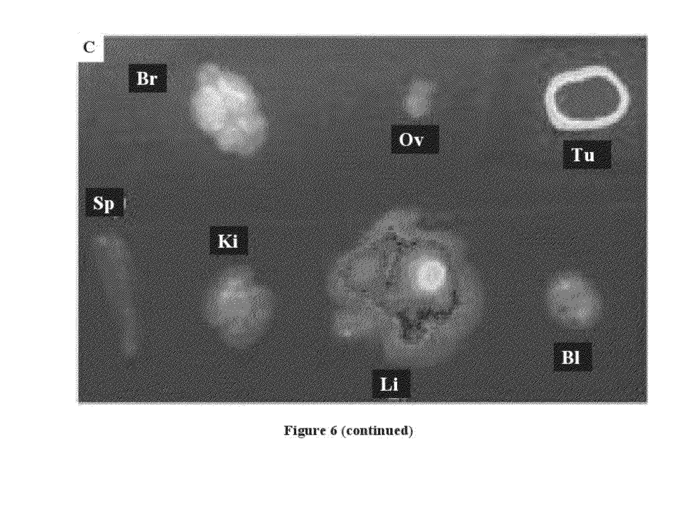

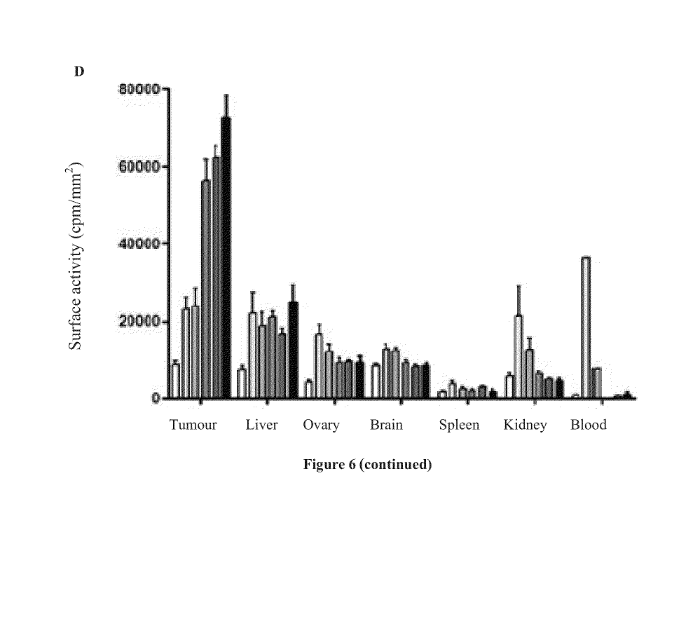

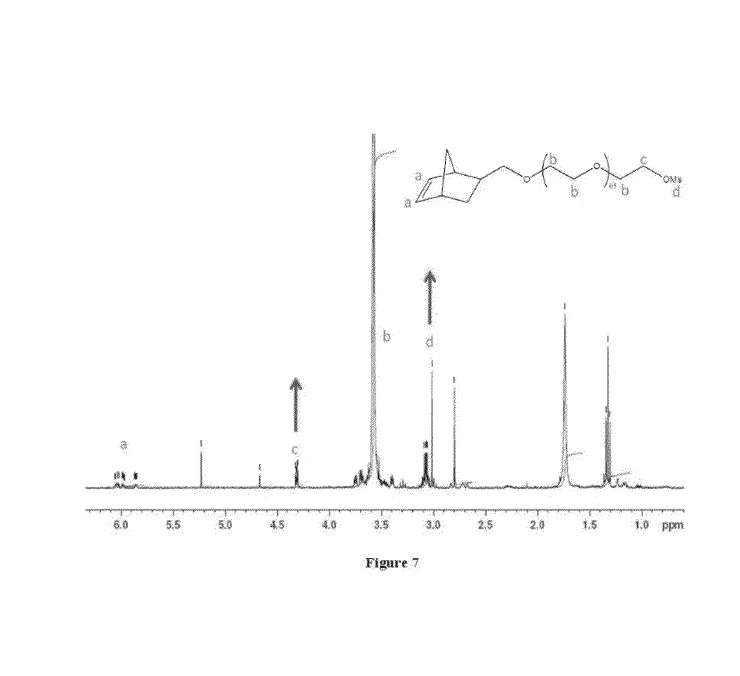

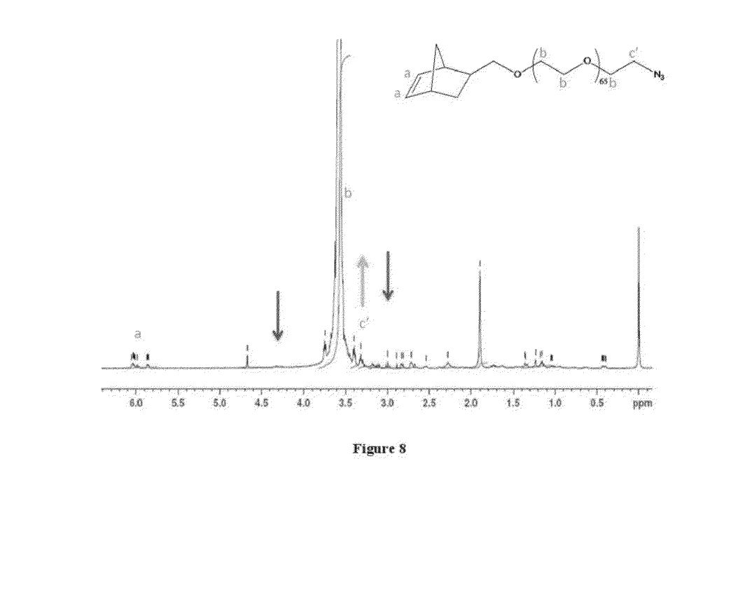

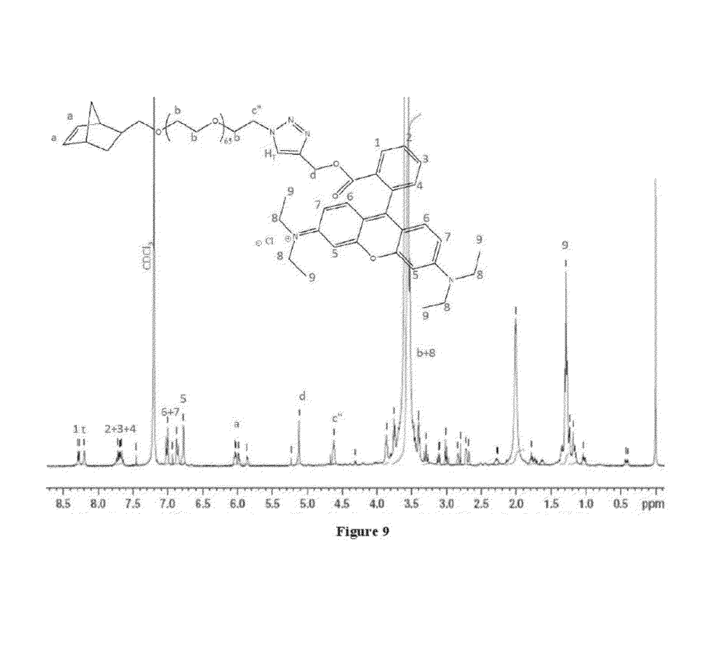

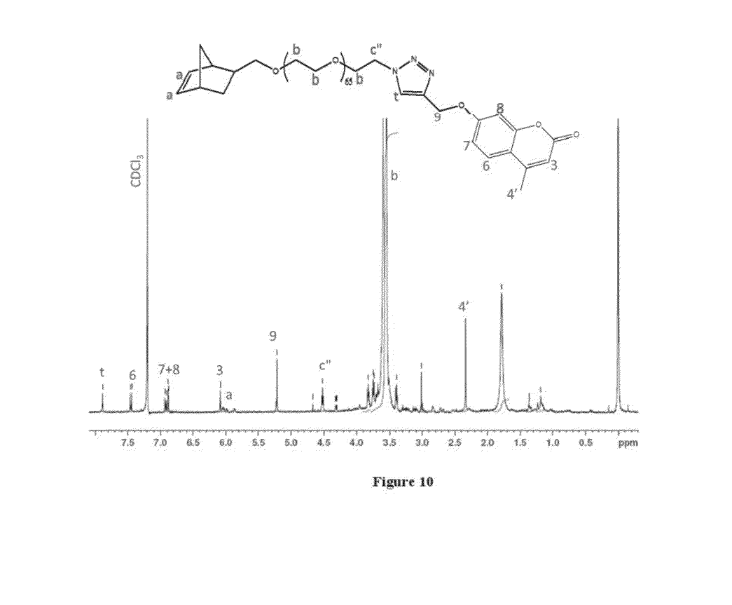

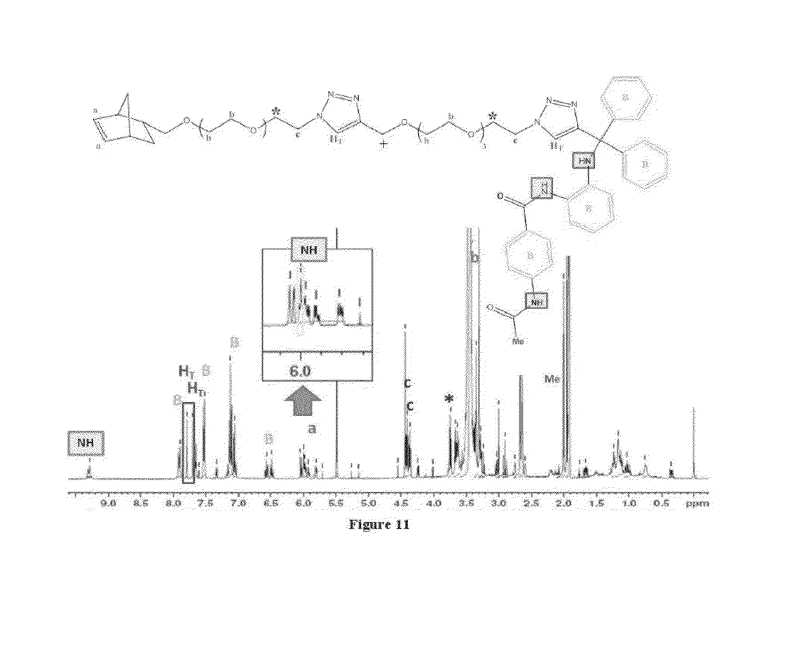

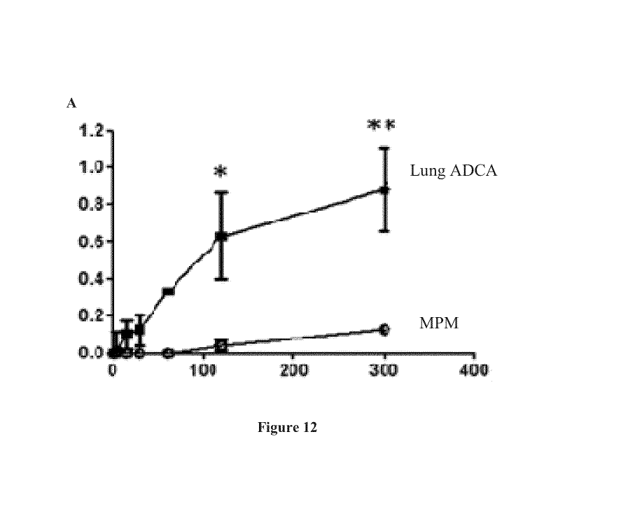

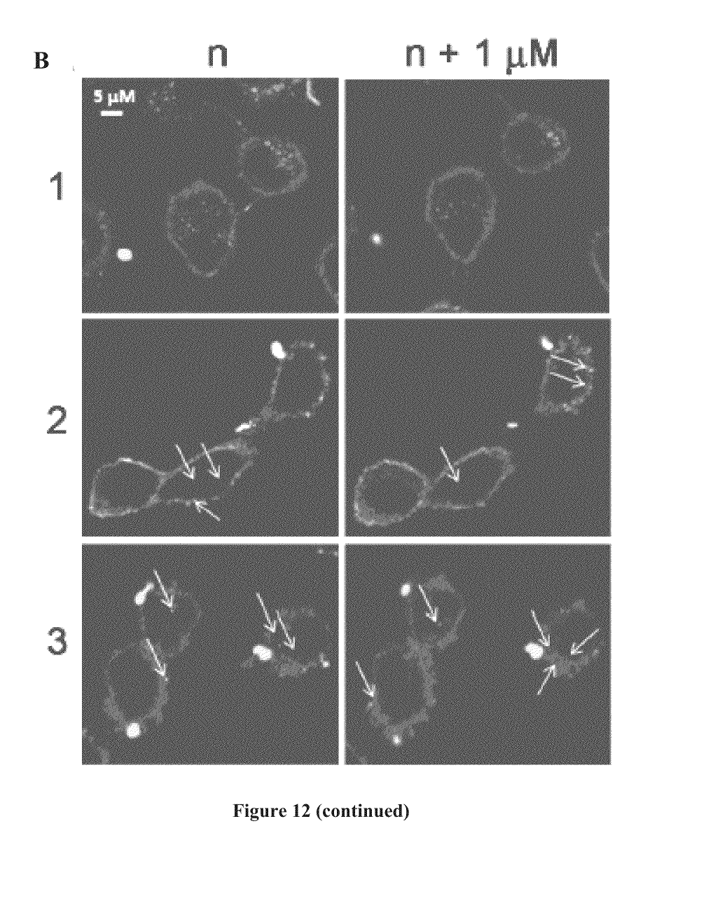

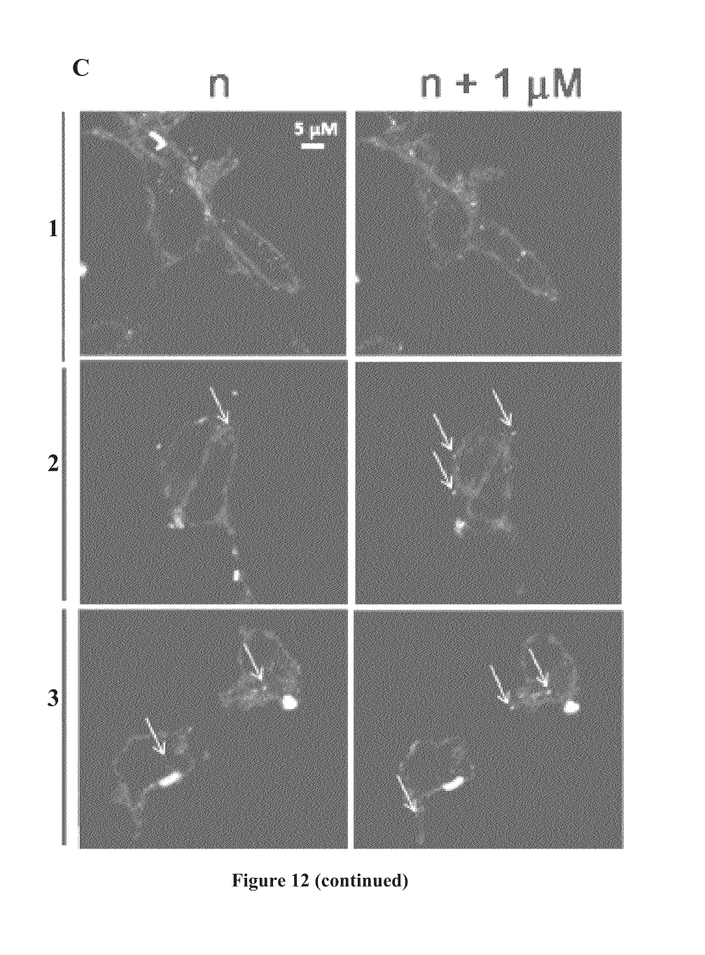

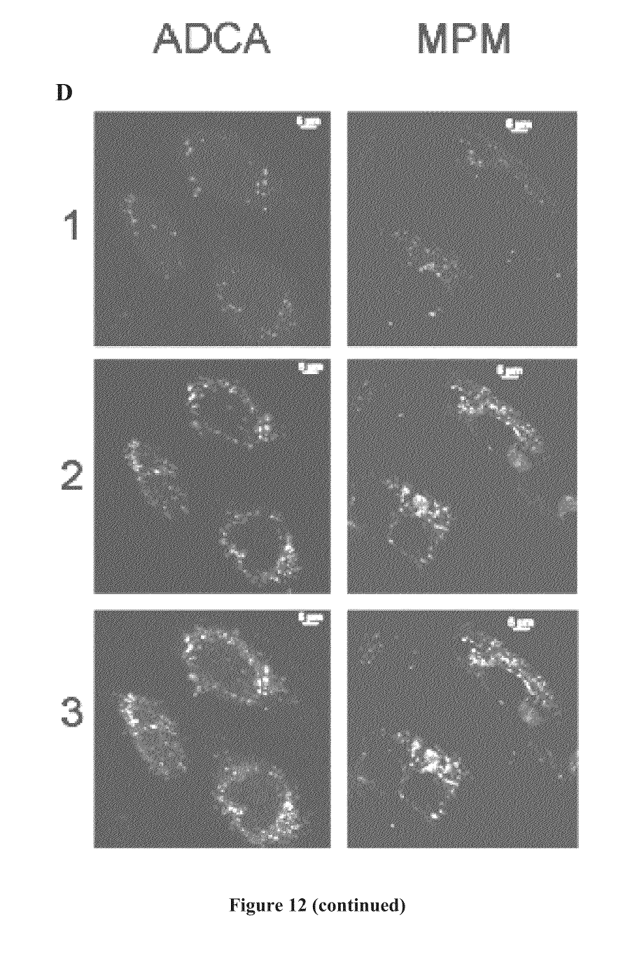

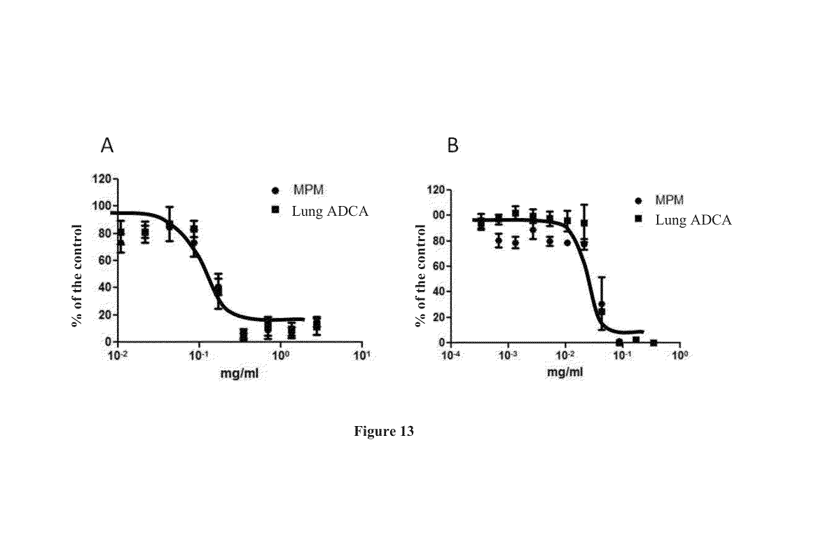

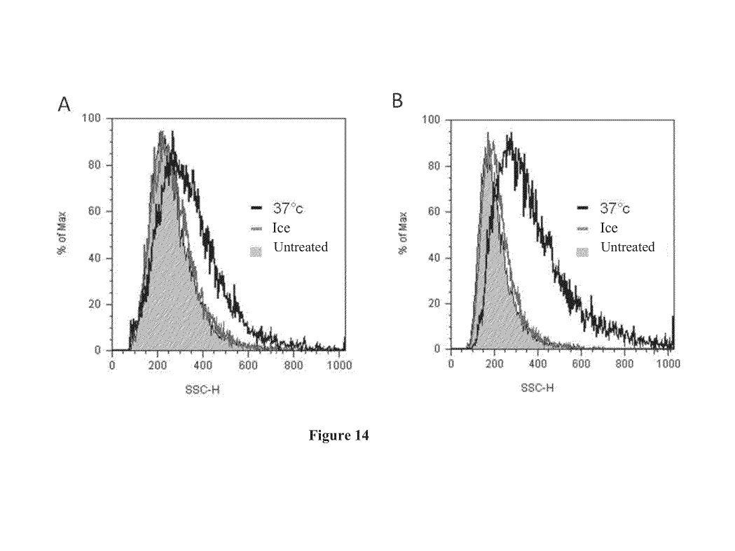

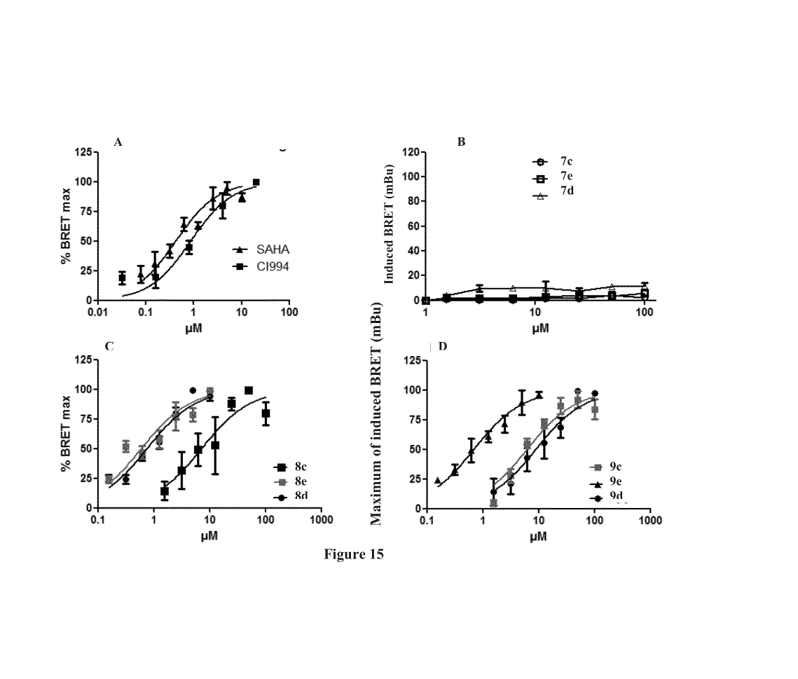

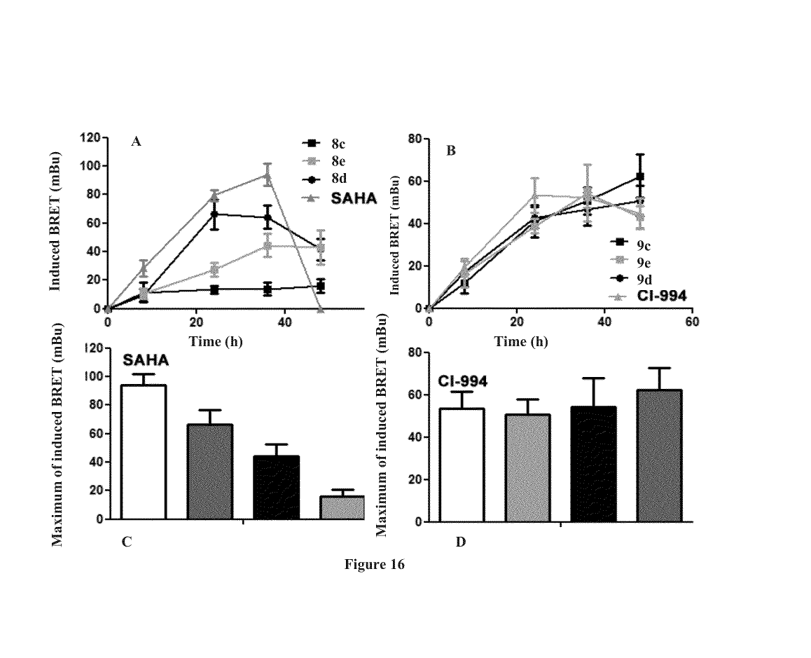

Briefly, the nanovector previously administered by intravenous route to a mammal, after circulation in the blood vessels for several hours, is trapped by EPR at the level of the endothelial cell and then internalized by endocytosis promoted by the peptide CPP leading to internalization of the nanovector in an endosome where the active ingredient is partly released because of the pH of 6 existing in the endosome. Maturation of this endosome leads to a lysosome in which the pH of 4-5 finalizes the hydrolysis of the active ingredient and leads to release of the active ingredient in the cytoplasm.

Early exit of the endosome can occur for compounds that are sensitive to the acid environment, which can penetrate the nucleus by means of the nuclear pore complex (NPC) mechanism based on the nuclear localization signals (NLS).

FIG. 2 shows the two possible alternatives for synthesis of the compounds of the invention:
Bioconjugation step (click) and then a step of ring-opening polymerization (ROMP), or
step of ring-opening polymerization (ROMP) and then bioconjugation step (click).

FIGS. 3A and 3B show the curves of hydrolysis in an acid environment obtained with the compound 9d of Example 5 of the invention at pH 4.3, 5.3 and 7.3.

FIG. 3A: Black square (dotted line: pH 4.3; Solid black circles (solid line): pH 5.3.

x-axis: time in minutes
y-axis: % hydrolysis

FIG. 3B: Black square: pH 7.3.

x-axis: time in hours
y-axis: % hydrolysis

FIGS. 4A to 4D show the size of the particles of the invention measured by dynamic light scattering (DLS) and transmission electron microscopy (TEM).

FIG. 4A: DLS: Size distribution by intensity
solid line: compound 16a (Example 14 Diagram IV-1)
dotted line: compound 16b (Example 14 Diagram IV-2)

FIG. 4B: TEM compound 16b (Example 14 Diagram IV-2)

FIG. 4C: TEM compound 16a (Example 14 Diagram IV-1)

FIG. 4D: TEM compound 16a (Example 14 Diagram IV-1)
x-axis: size (diameter in nm)
y-axis: intensity (%)

The particles of compound 16a (Example 14 Diagram IV-1) have a size of about 300 nm and the particles of compound 16b (Example 14 Diagram IV-2) have a size of about 400 nm.

FIGS. 5A to 5C show the co-localization of the particles of the invention (compound 16a) of Example 14 (Diagram IV) with the lysosomes after internalization of the detecting probe in the cell.

FIG. 5A: Particles (compound 16a) detected by fluorescence.

FIG. 5B: Acidic lysosomal vesicles revealed by labelling with an anti-LAMP antibody

FIG. 5C: Superposition of 5A and 5B showing co-localization of the particles with the lysosomes.

FIGS. 6A to 6D show the selective targeting of tumours (malignant peritoneal mesothelioma cells (AK7)) by the particles (compound 16a of the invention).

FIG. 6A: Mouse with a subcutaneous tumour (AK7) at the level of the lower back, on the left. On the plate corresponding to the whole animal, fluorescence is seen only at the level of the tumour at 24h.

FIG. 6B: The photograph corresponds to the isolated tumour and to several dissected organs (spleen at top right, the 2 kidneys bottom left and the liver bottom right). Fluorescence is observed only at the level of the tumour and not in the spleen or the kidneys. The very weak residual signal detected in the liver corresponds to necessary passage of the nanovectors of the invention without a retention effect.

FIG. 6C: One week after injection, fluorescence is observed at the level of the dissected tumour (Tu), the liver (Li), the ovaries (Ov), the brain (Br), the spleen (Sp) and the kidneys (Ki) as well as in the blood (Bl) one week after injection.

FIG. 6D: graphical representation of the fluorescence intensities measured in the various organs at the indicated times post-injection. Y-axis: surface activity in cpm/mm².

X-axis (from left to right): Tumour, Liver, Ovary, Brain, Spleen, Kidney and blood.

FIG. 7 shows the NMR spectrum in CDCl₃ of the compound NB-PEO-OMs (12).

FIG. 8 shows the NMR spectrum in CDCl₃ of the compound NB-PEO-N₃ (13).

FIG. 9 shows the NMR spectrum in CDCl₃ of the compound NB-PEO-Rhodamine B (15a).

FIG. 10 shows the NMR spectrum in CDCl₃ of the compound NB-PEO-Coumarin (15b).

FIG. 11 shows the NMR spectrum in CDCl₃ of the compound NB-PEO-CI-994 (17c).

FIGS. 12A to 12D show the cell penetration of the compounds of the invention (compound 16a of Example 14, Diagram IV) and the associated cell trafficking

FIG. 12A: kinetics of endocytosis of compound 16a by cells of malignant pleural mesothelioma (MPM: cell line Meso 13) and of lung adenocarcinoma (ADCA: cell line 153).

1.10³ cells of MPM or of lung adenocarcinoma are incubated with 0.43 μg of compound 16a at different times. Fluorescence was used for measuring the internalization using a fluorometer. The results are expressed as an average value±standard deviation of the results obtained on three different cell lines from MPM or lung ADCA.

* p<0.05 and ** p<0.01.

Y-axis: Particles internalized by endocytosis (μg/10³ cells)

X-axis: time (min)

Based on the fluorescence, the quantity of compound 16a internalized per quantity of cells after 120 minutes' incubation is 0.042±0.028 μg/1.10³ cells in the case of MPM and 0.629±0.231 μg/1.10³ cells in the case of ADCA, showing capacity for internalizing compound 16a increased by a factor of 15 of the ADCA cells relative to the MPM cells. After 300 minutes, this ratio is reduced to a factor of 7 (MPM: 0.125±0.016 μg/1.10³ cells and ADCA: 0.881±0.226 μg/1.10³ cells).

FIG. 12B: Electron microscopy of the endocytosis of compound 16a by the ADCA cells.

The columns n and n+1 μM represent the layer n and the layer n+1μ.

Lines 1, 2, 3: treatments at 37° C., 4° C. and cytochalasin respectively.

The arrows indicate the localization of compound 16a.

FIG. 12C: Electron microscopy of the endocytosis of compound 16a by the MPM cells.

The columns n and n+1 μM represent layer n and layer n+1μ.

Lines 1, 2, 3: treatments at 37° C., 4° C. and cytochalasin respectively.

The arrows indicate the localization of compound 16a.

FIG. 12D: co-localization with the intracellular acidic compartments (column on left ADCA, and column on right MPM)

Lines 1, 2 and 3: fluorescence of compound 16a, labelling by Lamp-1 and fusion of lines 1 and 2 respectively.

FIGS. 12B and 12C, columns n and n+1 μM show the presence of several points delimited by fine membrane labelling. These figures demonstrate the internalization of compound 16a in the cells at 37° C. (line 1). When the cells are incubated with ice (line 2) or with cytochalasin D (line 3) before adding compound 16a, compound 16a is mainly localized on the membranes and not within the cells. This suggests that internalization of the compounds of the invention requires an active mechanism involving an actin network.

FIGS. 13A and 13B show the cell toxicity of the particles of the invention (compound 19a, Example 14, Diagram IV-1) by determination of the dose-response curves on MPM or ADCA cells.

FIG. 13A: dose-response curve obtained with bare particles of the invention (without rhodamine)

FIG. 13B: dose-response toxicity obtained with particles 16a.

All the cells were kept in RPMI medium (Invitrogen) enriched with L-glutamine (2 mM), penicillin (100 IU/ml), streptomycin (0.1 mg/ml) and heat-inactivated 10% foetal calf serum (FCS) (Eurobio).

The cells were incubated with increasing doses of bare particles or of particles of the invention coupled with rhodamine for 72 h. Cell growth was evaluated with a Uptiblue cell counting reagent (Interchim). Reduction of this compound by the cells leads to the formation of a fluorescent compound that is quantified by measuring the fluorescence at 595 nM after excitation at 532 nM using Typhoon apparatus (GE Healthcare). The cells were seeded in 96-well plates at a density of 5×10³ cells/well in a culture medium. After 24 h, Uptiblue (5%, v/v) was added to the culture medium for 2.5 h at 37° C.

The fluorescence was measured and was referred to the number of cells on day D=0. The culture medium containing Uptiblue was replaced with a medium containing or not containing the particles of the invention for 72 h. Uptiblue was added to the culture medium for 2.5 h at 37° C. The fluorescence was measured as described above and was referred to the number of cells on day 3. Cell growth was defined as the ratio of the intensity of fluorescence on day D=0 to the intensity of fluorescence on day D=3.

The results are presented as an average value±standard deviation of the determinations carried out on at least three different cell lines of MPM (Meso 4, Meso 13, Meso 34, Meso 56, Meso 76 or Meso 95B) or ADCA (ADCA 3, ADCA 72, ADCA 117 or ADCA 153).

The bare particles and the particles coupled with rhodamine show similar toxicity on all the cell lines tested with an IC₅₀ of 0.34 mg/ml±0.03 in the case of the bare particles and of 0.031 mg/ml±0.004 in the case of the particles 16a.

The toxicity of the particles shows a very slight variation as a function of the cell lines. This suggests the involvement of a physical phenomenon to explain the toxicity on the cells and not pathways dependent on cell death that would probably include intrinsic sensitivity of the cell lines and then a variation in the response.
The slight difference between the bare particles and the particles with rhodamine (16a) may be due to the change in hydrophilicity at the surface of the particles.

FIGS. 14A and 14B show the potential for internalization of the particles by flow cytometry.

This method allows individual analysis of the cells by measuring the absorption of light (FSC) and light scattering (SSC). The increase in cell grain size changes the light scattering properties of the cells and increases the SSC values.

ADCA 153 (FIG. 14A) and MMP (Meso 13, FIG. 14B) cells were incubated with 3.45 mg/ml of bare nanoparticles of the invention for 2 h with ice (0° C.) or at 37° C.

FIGS. 14A and 14B clearly show that the SSC of the cells was increased when incubation of the cells was carried out at 37° C. but not at 0° C. The increase in the SSC of the cells treated with nanoparticles reflects an increase in cell grain size due to internalization of the particles and not to deposition of particles on the cell membranes as indicated by the results at 0° C.

The presence of internalization of the particles only at 37° C. demonstrates the involvement of an active endocytosis mechanism.
In both figures:
The curve on the right corresponds to a temperature of 37° C.
The curve on the left corresponds to a temperature of 0° C. superimposed on the control.

FIGS. 15A to 15D present pharmacological characterization using BRET for the free active ingredients (SAHA, CI-994), their derivatives 8 (c, d, e) and 9 (c, d, e) and the expected alcohols released 7 (c, d, e).

The results are the average value±standard deviation of three independent experiments.
The EC₅₀ (μM) values obtained are as follows:
SAHA alone: 0.42±1.14
9c: 7.17±1.11
9d: 0.59±1.17
9e: 0.42±1.14

### CI-9947c: 0.86±1.14

9c: 6.20±1.15
9d: 8.88±1.19
9e: 0.68±1.13
These results show that the iHDACs are released, and the prodrugs do not themselves inhibit HDAC.

FIG. 15A: SAHA and CI-994

FIG. 15B: compounds 7c, d, e

FIG. 15C: compounds 8c, d, e

FIG. 15D: compounds 9c, d, e

FIGS. 16A to 16D show the kinetics of restoration of inhibition of HDAC measured by BRET.

Evaluation of the kinetics of induction of acetylation of the histones using BRET:

FIG. 16A: SAHA and compounds 8c, d, e

FIG. 16B: CI-994 and compounds 9c, d, e

FIGS. 16C and 16D: Maximum BRET induced as a function of the different compounds:

16C: from left to right: SAHA, 8d, e, c

16D: from left to right: CI-994, 9d, e, c

### EXAMPLES

### Chemical Section

DCM: dichloromethane; ACN: acetonitrile; DMF: dimethylformamide; THF: tetrahydrofuran.

CDCl₃: Deuterated chloroform

### A) Synthesis of the Units for Click Chemistry

### Example 1

### Preparation of the Acid-Sensitive Arms: Compound 7c and 7d

Compounds 7c (R₂ and R′₂═PH) and 7d (R₂ and R′₂=4-OMe), 7e (R₂ and R′₂=4-Me), 7f (R₂ and R′₂=2,4-diOMe), 7 g (R₂ and R′₂=4-F), 7 h (R₂ and R′₂=4—Cl), are prepared according to the following Diagram I (throughout the experimental section, the letters c to h have the same meaning)

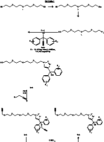

### 1.1: Preparation of 2-(2-(2-(2-azidoethoxy)ethoxy)ethoxy)ethanol (3)

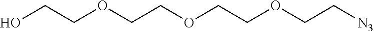 [SMILES: [N-]=[N+]=NCCOCCOCCOCCO]

NaN₃ (0.938 g; 14.43 mmol; 5 eq.) is added at ambient temperature to a solution of 2 (1.0 g; 2.89 mmol; 1 eq.) in 20 mL of DMF. The solution is stirred for 2 days and diluted with DCM and washed with water and then with a saturated NaCl solution. The organic phase is dried over MgSO₄, filtered and concentrated under vacuum. The residue is purified (flash chromatography, silica, eluent DCM/MeOH) to give the nitride 3 in the form of a colourless viscous oil (0.501 g; 2.29 mmol; 79%).

Rf (SiO₂, DCM/MeOH (95/5)): 0.33,

¹H NMR (CDCl₃, 400 MHz): δ=3.75 (t, 2H, J=4.1 Hz), 3.70 (m, 10H), 3.64 (dd, 2H, J=3.8 Hz, J=5.1 Hz), 3.42 (t, 2H, J=5.0 Hz), 2.61 (bs, 1H).

### 1.2: Preparation of 2-(2-(2-(2-(4-(hydroxydiphenylmethyl)-1H-1,2,3-triazol-1-yl)ethoxy)ethoxy)ethoxy)ethanol (5c)

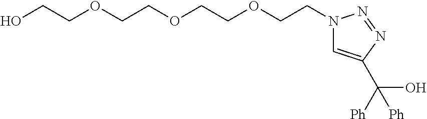 [SMILES: OCCOCCOCCOCCn1cc(C(O)(c2ccccc2)c2ccccc2)nn1]

The following are added to 100 mL of DCM/H₂O mixture (1/1): 3 (2.015 g; 9.19 mmol; 1 eq.), 4c prepared according to Cadierno, V.; Francos, J.; Gimeno, J. Tet. Lett. 2009, 50, 4773 (1.914 g; 9.19 mmol; 1 eq.) and CuBr (0.264 g; 1.838 mmol; 0.2 eq.). The solution is stirred vigorously for 20 h and then extracted with DCM and washed with a saturated solution of NH₄Cl. The organic phase is dried over MgSO₄, filtered and concentrated under vacuum. The crude product is purified (flash chromatography, silica gel, eluent DCM/MeOH) to give 5c in the form of an oil (3.317 g; 7.76 mmol; 84%).

Rf (SiO₂, DCM/MeOH (95/5)): 0.19,

¹H-NMR (acetone-d₆, 400 MHz): δ=7.73 (s, 1H), 7.47-7.44 (m, 4H), 7.31-7.26 (m, 4H), 7.25-7.20 (m, 2H), 5.34 (s, 1H), 4.56 (t, 2H, J=5.21 Hz), 3.89 (t, 2H, J=4.20 Hz), 3.60-3.56 (m, 5H), 3.54-3.50 (m, 6H), 3.49-3.46 (m, 2H).

¹³C-NMR (acetone-d₆, 100 MHz): δ=154.75, 148.12, 128.41, 128.16, 127.69, 124.38, 77.19, 73.47, 71.21, 71.17, 71.09, 70.17, 61.98, 50.68,

### 1.3: Preparation of 2-(2-(2-(2-(4-(hydroxybis(4-methoxyphenyl)methyl)-1H-1,2,3-triazol-1-yl)ethoxy)ethoxy)ethoxy)ethanol (5d)

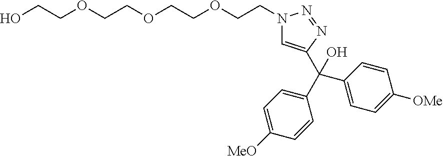 [SMILES: COc1ccc(C(O)(c2ccc(OC)cc2)c2cn(CCOCCOCCOCCO)nn2)cc1]

The following are added to 100 mL of DCM/H₂O mixture (1/1): 3 (1.988 g; 9.07 mmol; 1 eq.), 4d prepared according to Gabbutt, C. D.; Heron, B. M.; Instone, A. C.; Thomas, D. A. Partington, S. M.; Hursthouse, M. B.; Gelbrich, T. Eur. J. Org. Chem. 2003, 7, 1220; (2.379 g; 9.07 mmol; 1 eq.) and CuBr (0.260 g; 1.814 mmol; 0.2 eq.). The solution is stirred vigorously for 20 h and extracted with DCM and washed with a saturated solution of NH₄Cl. The organic phase is dried (MgSO₄), filtered and concentrated under vacuum. The crude product is purified (flash chromatography, silica gel, eluent DCM/MeOH) to give 5d in the form of an oil (3.463 g; 7.19 mmol; 79%).

Rf (SiO₂, DCM/MeOH (95/5)): 0.15,

### 1.4: Preparation of 2-(2-(2-(2-(4-(hydroxybis(4-fluorophenyl)-1H-1,2,3-triazol-1-yl)ethoxy) ethoxy)ethoxy)ethanol (5g)

Preparation of Compound (4g):

To a mixture of 1.50 ml of trimethylsilyl acetylene (10.31 mmol; 1.5 eq.) in 30 ml of anhydrous THF, gradually add, at −10° C., 6.45 ml of BuLi (1.6M) (10.31 mmol; 1.5 eq.). Stir the mixture at −10° C. for one hour. Then, keeping the temperature the same, add a mixture of 1.50 g of difluorobenzophenone (6.87 mmol; 1 eq.) diluted in 10 ml of anhydrous THF. Stir for 5 h at −10° C.

Allow the temperature to return to 0° C., then add a solution of 0.58 g of KOH diluted in 6 ml of distilled methanol. Stir the mixture at ambient temperature for 12 h.

Add a solution of acetic acid to the mixture until the pH=7. Pour the mixture into a solution of NaCl (150 ml). Extract the organic phases 3×100 ml of ethyl acetate. The organic phases are then dried over MgSO₄, filtered and then evaporated under vacuum.

The mixture is purified by flash chromatography: EP/EtOAc (from 0% to 10% of EtOAc).

1.66 g of product (4g) is obtained in the form of a yellow oil.

Yield: 98%

Rf (EP/EtOAc:80/20):0.58

¹H NMR (CDCl₃, 400 MHz) δ (ppm): 2.90 (s, 1H); 7.00 (t, 4H, J=8 Hz); 7.54 (dd, 4H, J=8 Hz)

¹³C NMR (ACETONE D⁶, 75.4 MHz) δ (ppm): 73.4; 76.8; 87.5; 115.4; 115.6; 128.8; 142.8; 142.9; 161.6; 164.1

¹⁹F NMR (ACETONE D⁶, 400 MHz) δ (ppm): −117.6

Preparation of Compound (5g):

At ambient temperature, add 0.36 g of the azide 3 (1.63 mmol; 1 eq.) to a mixture of 0.40 g of compound (4g) in 30 ml of DCM/H₂O (1/1). Then add 46.00 mg of copper bromide (0.33 mmol; 0.2 eq.). Stir the mixture for 12 h at ambient temperature.

Add 20 ml of H₂O to the mixture. Extract the organic phases 3×50 ml of DCM. Wash the organic phases with a saturated solution of NH₄Cl. Then the organic phase is dried, filtered and then evaporated.

0.75 g of the product of compound (5g) is obtained without purification.

Yield: Quantitative.

Rf (DCM/MeOH:90/10): 0.45

¹H NMR (ACETONE D⁶, 400 MHz) δ (ppm): 3.48 (m, 12H); 3.88 (t, 2H, J=8 Hz); 4.55 (t, 2H, J=8 Hz); 5.55 (s, 1H); 7.01 (t, 4H, J=8 Hz); 7.46 (dd, 4H, J=8 Hz); 7.80 (s, 1H)

¹³C NMR (ACETONE D⁶, 75.4 MHz) δ (ppm): 61.9; 71.0; 71.1; 71.2; 73.4; 114.9; 115.2; 130.1

¹⁹F NMR (ACETONE D⁶, 400 MHz) δ (ppm): −117.6

### 1.5: Preparation of 2-(2-(2-(2-(4-(hydroxybis(4-chlorophenyl)-1H-1,2,3-triazol-1-yl)ethoxy) ethoxy)ethoxy)ethanol (5h)

Preparation of Compound (4h):

For the synthesis of (4h), follow the same procedure as for the synthesis of compound (4g). 3.53 g of dichlorobenzophenone (14.05 mmol; 1 eq.); 13.17 ml of BuLi (1.6M) (21.08 mmol; 1.5 eq.); 3.00 ml of TMSA (21.08 mmol; 1.5 eq.); 1.18 g of KOH (21.08 mmol; 1.5 eq.) in 12 ml of dry methanol; 2×60 ml of anhydrous THF. 2.50 g of product 4h is obtained in the form of a clear oil.

Yield: 64%

Rf (EP/EtOAc: 95/5): 0.25

¹H NMR (ACETONE D⁶, 400 MHz) δ (ppm): 3.46 (s, 1H); 6.00 (s, 1H); 7.36 (d, 4H, J=8 Hz); 7.61 (d, 4H, J=8 Hz)

¹³C NMR (ACETONE D⁶, 75.4 MHz) δ (ppm): 73.4; 77.1; 86.9; 128.5; 128.9; 133.7; 145.4

Preparation of Compound (5h):

Same procedure as for the synthesis of compound (5g).

0.93 g of (4g) (3.34 mmol; 1 eq.); 0.73 g of the azide 3 (3.34 mmol; 1 eq.); 95.00 mg of CuBr (0.67 mmol; 0.2 eq.); 50 ml DCM/H₂O(1/1).

1.56 g of product (5h) is obtained in the form of a yellow oil.

Yield: 95%

Rf (DCM/MeOH:90/10): 0.33

¹H NMR (ACETONE D⁶, 400 MHz) δ (ppm): 3.52 (m, 12H); 3.88 (t, 2H, J=8 Hz); 4.55 (t, 2H, J=8 Hz); 5.68 (s, 1H); 7.33 (d, 4H, J=8 Hz); 7.46 (d, 4H, J=8 Hz); 7.83 (s, 1H)

¹³C NMR (ACETONE D⁶, 75.4 MHz) δ (ppm): 61.9; 70.0; 70.1; 73.4; 128.55; 129.86

### 1.6: Preparation of (1-(3,6,9,12-tetraoxapentadec-14-ynyl)-1H-1,2,3-triazol-4-yl)diphenyl methanol (7c)

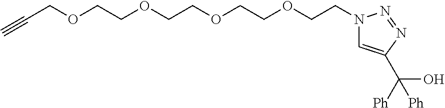 [SMILES: C#CCOCCOCCOCCOCCn1cc(C(O)(c2ccccc2)c2ccccc2)nn1]

At 0° C., NaH (0.232 g; 5.8 mmol; 2 eq.; 60% by weight in the oil) is added to 100 mL of dry THF containing 5c (1.240 g; 2.90 mmol; 1 eq.). The reaction mixture is stirred for 2 hours and propargyl bromide (0.313 mL; 2.90 mmol; 1 eq.) in toluene (80% by weight) is added slowly. The reaction mixture is stirred for 2 days, allowing the temperature to rise. The reaction is stopped by adding saturated NaCl solution at 0° C. and it is extracted with DCM. The organic phase is dried (MgSO₄), filtered and concentrated under vacuum. The oil obtained is purified (flash chromatography, silica gel, eluent DCM/MeOH) to give the expected ether 7c (0.774 g; 1.66 mmol; 57%) in the form of a viscous oil. A proportion of the diol 5c is converted to the form of diether 6c, which when treated for 12 h with a 1M ACN/KHSO₄ solution gives the monoether 7c completely.

Rf (SiO₂, DCM/MeOH 95:5): 0.53

¹H-NMR (acetone-d₆, 400 MHz): δ=7.72 (s, 1H), 7.48-7.44 (m, 4H), 7.31-7.27 (m, 4H), 7.25-7.21 (m, 2H), 5.27 (s, 1H), 4.56 (t, 2H, J=5.2 Hz), 4.15 (d, 2H, J=2.4 Hz), 3.90 (t, 2H, J=5.2 Hz), 3.61-3.56 (m, 6H), 3.54-3.50 (m, 6H), 2.92 (t, 1H, J=2.4 Hz).

¹³C-NMR (acetone-d₆, 100 MHz): δ=154.73, 148.12, 128.41, 128.16, 127.70, 124.34, 80.95, 77.20, 75.75, 71.22, 71.21, 71.19, 71.10, 70.97, 70.17, 69.81, 58.57, 50.70,

### 1.7: Preparation of (1-(3,6,9,12-tetraoxapentadec-14-ynyl)-1H-1,2,3-triazol-4-yl)bis(4-methoxyphenyl) methanol (7d)

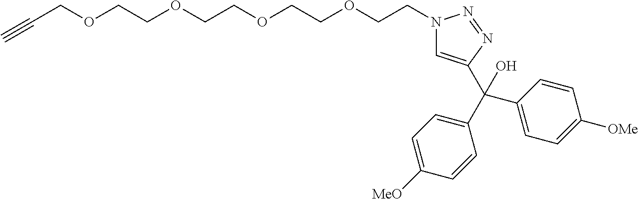 [SMILES: C#CCOCCOCCOCCOCCn1cc(C(O)(c2ccc(OC)cc2)c2ccc(OC)cc2)nn1]

At 0° C., NaH (0.274 g; 6.46 mmol; 2 eq.; 60% by weight in the oil) is added to 100 mL of dry THF containing 5d (1.674 g; 3.43 mmol; 1 eq.). The reaction mixture is stirred for 2 hours and propargyl bromide (0.370 mL; 3.43 mmol; 1 eq.) in toluene (80% by weight) is added slowly. The reaction mixture is stirred for 2 days, allowing the temperature to rise. The reaction is stopped at 0° C. by adding saturated NaCl solution and is then extracted with DCM. The organic phase is dried (MgSO₄), filtered and concentrated under vacuum. The oil obtained is purified (flash chromatography, silica gel, eluent DCM/MeOH) to give the expected product 7d in the form of a viscous oil (1.276 g; 2.43 mmol; 71%). The diether 6d also obtained is converted to monoether 7d by treatment with ACN/KHSO₄ 1M.

Rf (SiO₂, DCM/MeOH 95:5): 0.28

¹H-NMR (acetone-d₆, 400 MHz): δ=7.68 (s, 1H), 7.33-7.31 (m, 4H), 6.85-6.82 (m, 4H), 5.09 (s, 1H), 4.54 (t, 2H, J=5.2 Hz), 4.15 (d, 2H, J=2.4 Hz), 3.88 (t, 2H, J=5.2 Hz), 3.76 (s, 6H), 3.60-3.55 (m, 6H), 3.54-3.51 (m, 6H), 2.93 (t, 1H, J=2.4 Hz).

¹³C-NMR (acetone-d₆, 100 MHz): δ=159.53, 155.34, 140.54, 129.38, 124.11, 113.64, 80.97, 76.65, 75.81, 71.22, 71.19, 71.10, 70.96, 70.18, 69.81, 58.59, 55.51, 50.66,

### 1.8: Preparation of (1-(3,6,9,12-tetraoxapentadec-14-ynyl)-1H-1,2,3-triazol-4-yl)bis(4-paramethylphenyl) methanol (7e)

Same procedure as for the synthesis of compound (7d).

yellow oil (1.51 g, 3.04 mmol, 82%). TLC MeOH/DCM 5/95 R_{f}=0.23; ¹H NMR (400 MHz, Acetone-D₆): δ=2.29 (s, 6H), 2.93 (t, 1H, J=2.4 Hz), 3.49-3.61 (m, 12H), 3.87 (t, 2H, J=5.3 Hz), 4.15 (d, 2H, J=2.4 Hz), 4.53 (t, 2H, J=5.1 Hz), 5.14 (br s, 1H), 7.09 (d, 4H, J=8.5 Hz), 7.32 (d, 4H, J=8.5 Hz), 7.68 (s, 1H); ¹³C NMR (100 MHz, Acetone-D₆): δ=154.9, 145.3 (2C), 136.9 (2C), 128.9 (4C), 128.0 (4C), 124.2, 80.9, 76.9 (2C), 75.8, 71.9, 71.1, 71.1, 71.0, 70.1, 69.7, 58.5, 50.6, 21.0 (2C); HRESI-MS: m/z 516.2473 (calcd. for C₂₈H₃₅N₃O₅Na 516.24744 [M+Na]⁺)

### 1.9: Preparation of (1-(3,6,9,12-tetraoxapentadec-14-ynyl)-1H-1,2,3-triazol-4-yl)bis(4-fluorophenyl) methanol (7g)

To a mixture of 0.76 g of product (5g) (1.55 mmol; 1 eq.) in 50 ml of anhydrous THF, gradually add, at 0° C., 0.12 g of NaH (60%) (3.11 mmol; 2 eq.). The mixture is stirred, keeping the temperature the same, for 2 h. Then add 1.38 ml of propargyl bromide (80%) (1.55 mmol, 1 eq.). Stir the mixture at ambient temperature for 24 h.

At 0° C., add 50 ml of saturated NaCl solution to the mixture. The organic phase is extracted 1×100 ml of DCM. The organic phase is then dried, filtered and then evaporated under vacuum.

Add 100 ml of a solution of KHSO₄(1M)/ACN to the crude mixture and stir at ambient temperature for 12 h.

Extract 1×100 ml of DCM. The organic phase is then dried, filtered and then evaporated under vacuum.

The crude mixture is purified by combi flash: DCM/MeOH (from 0% to 5% of MeOH).

0.302 g of product (7g) is obtained in the form of a yellow oil.

Yield: 39%

Rf (DCM/MeOH:95/5): 0.36

¹H NMR (ACETONE D⁶, 400 MHz) δ (ppm): 2.92 (s, 1H); 3.54 (m, 12H); 3.89 (t, 2H, J=8 Hz); 4.15 (s, 2H); 4.55 (t, 2H, J=8 Hz); 5.49 (s, 1H); 7.04 (t, 4H, J=8 Hz); 7.45 (dd, 4H, J=8 Hz) 7.79 (s, 1H)

¹³C NMR (ACETONE D⁶, 75.4 MHz) δ (ppm): 50.6; 58.5; 69.7; 70.0; 70.9; 71.0; 71.1; 75.7; 76.4; 80.8; 114.8; 115.1; 124.2; 129.9; 130.1; 143.9; 144.0; 154.4; 161.4; 163.8

¹⁹F NMR (ACETONE D⁶, 400 MHz) δ (ppm): −117.6

### 1.10: Preparation of (1-(3,6,9,12-tetraoxapentadec-14-ynyl)-1H-1,2,3-triazol-4-yl)bis(4-chlororophenyl)methanol (7h)

Same procedure as for the synthesis of compound (7g).

0.21 g of FE015-C1 (0.42 mmol; 1 eq.); 33.57 mg of NaH (60%) (0.82 mmol; 2 eq.); 45.00 μl of propargyl bromide (80%) (0.42 mmol; 1 eq.); 10 ml of anhydrous THF.

This gives 0.11 g of compound (7h) in the form of a yellow oil.

Yield: 49%

Rf (DCM/MeOH:95/5): 0.48

¹H NMR (ACETONE D⁶, 400 MHz) δ (ppm): 2.92 (s, 1H); 3.53 (m, 12H); 3.88 (t, 2H, J=8 Hz); 4.15 (s, 2H); 4.55 (t, 2H, J=8 Hz); 7.32 (d, 4H, J=8 Hz); 7.45 (d, 4H, J=8 Hz); 7.82 (s, 1H)

¹³C NMR (ACETONE D⁶, 75.4 MHz) δ (ppm): 50.7; 58.5; 69.7; 69.9; 70.8; 70.9; 71.0; 71.1; 75.7; 76.4; 80.9; 124.5; 128.5; 129.8; 133.3; 146.5; 153.8.

B) Preparation of the Compounds of Formula I in which R, Represents a Group of Formula (IV)

### Example 2

### Preparation of N¹-((1-(3,6,9,12-tetraoxapentadec-14-ynyl)-1H-1,2,3-triazol-4-yl)diphenylmethoxy)-N⁸-phenyloctanediamide (8c)

In this example, R₃ is an epigenetic modulator: SAHA

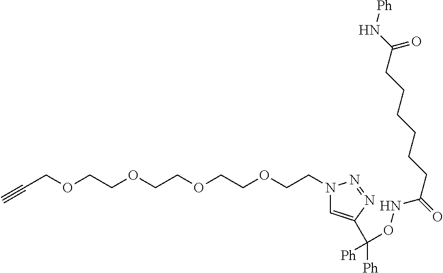 [SMILES: C#CCOCCOCCOCCOCCn1cc(C(ONC(=O)CCCCCCC(=O)Nc2ccccc2)(c2ccccc2)c2ccccc2)nn1]

The following are added to 5 mL of dry DCM under a nitrogen atmosphere: 7c (0.20 g; 0.43 mmol; 1 eq.) and a 2M HCl solution in ether (0.43 mL; 0.86 mmol, 2 eq.). The solution is refluxed for 2 hours and then the solvent is co-evaporated with toluene in order to give the intermediate chloride in the form of an oil. The preceding chloride dissolved in a minimum of ACN is added at ambient temperature to a solution of SAHA (0.340 g; 0.129 mmol, 3 eq.) in dry NEt₃ (0.24 mL, 1.72 mmol, 4 eq.) in 5 mL of ACN. The solution is stirred for 12 hours at ambient temperature. The solution is then filtered and washed with ACN to recover the remaining SAHA and the filtrate is concentrated under vacuum. Purification of the concentrate (automatic flash chromatography, silica gel, eluent DCM/EtOH/Et₃N) gives the prodrug 8c in the form of a colourless oil (0.052 g; 0.073 mmol; 17%). The alcohol 7c that remains is also recovered.

Rf (DCM/EtOH/Et3N 94:5:1)=0.33

¹H-NMR (acetone-d₆, 400 MHz): δ=9.75 (s, 1H), 9.08 (s, 1H), 7.69-7.65 (m, 3H), 7.46-7.41 (m, 4H), 7.34-7.25 (m, 8H), 7.04-7.00 (m, 1H), 4.57 (t, 2H, J=5.1 Hz), 4.16 (d, 2H, J=2.4 Hz), 3.88 (t, 2H, J=5.2 Hz), 3.62-3.55 (m, 6H), 3.53-3.49 (m, 6H), 2.93 (t, 2H, J=2.4 Hz), 2.31 (t, 2H, J=7.5 Hz), 1.93 (t, 2H, J=7.2 Hz), 1.60 (m, 2H), 1.39 (m, 2H), 1.25 (m, 2H), 1.12 (m, 2H).

### Example 3

### Preparation of N¹-((1-(3,6,9,12-tetraoxapentadec-14-ynyl)-1H-1,2,3-triazol-4-yl)bis(4-methoxyphenyl)methoxy)-N⁸-phenyloctanediamide (8d)

In this example, R₃ is an epigenetic modulator: SAHA

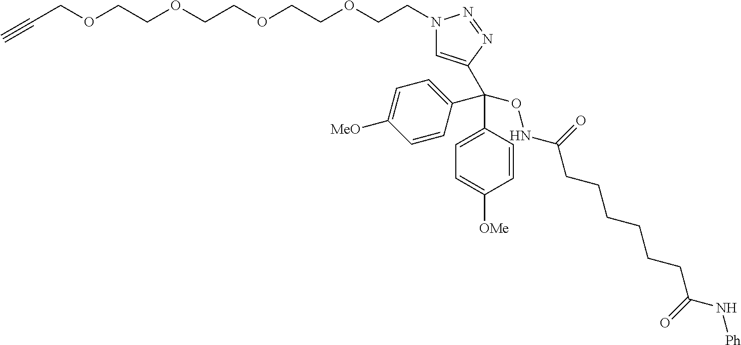 [SMILES: C#CCOCCOCCOCCOCCn1cc(C(ONC(=O)CCCCCCC(=O)Nc2ccccc2)(c2ccc(OC)cc2)c2ccc(OC)cc2)nn1]

The following are added to 5 mL of dry DCM under a nitrogen atmosphere: 7d 0.20 g (0.38 mmol; 1 eq.) and a 2M HCl solution in ether (0.38 mL; 0.76 mmol; 2 eq.). The solution turns red and is refluxed for 2 hours. The solution is then co-evaporated with toluene to give the intermediate chloride in the form of a red oil. The preceding chloride dissolved in a minimum of ACN is added at ambient temperature to a solution of SAHA (0.302 g; 1.14 mmol; 3 eq.) and dry NEt₃ (0.21 mL; 1.52 mmol; 4 eq.) in 5 mL of dry ACN. The red colour disappears after a few minutes and the solution is stirred for 12 hours. The solution is filtered and washed with CAN to recover the remaining SAHA and the filtrate is concentrated under vacuum. The concentrate is purified (automatic flash chromatography, silica gel, eluent DCM/EtOH/Et₃N) in order to give the prodrug 8d in the form of a viscous oil (0.054 g; 0.070 mmol; 18%). The alcohol 7d that remains is also recovered.

Rf (DCM/EtOH/Et₃N 94:5:1)=0.29

¹H-NMR (acetone-d₆, 400 MHz): δ=9.75 (s, 1H), 9.08 (s, 1H), 7.68-7.67 (m, 3H), 7.32-7.25 (m, 6H), 7.04-7.00 (m, 1H), 6.87-7.85 (m, 4H), 4.57 (t, 2H, J=5.1 Hz), 4.16 (d, 2H, J=2.4

Hz), 3.88 (t, 2H, J=5.2 Hz), 3.79 (s, 6H), 3.62-3.56 (m, 6H), 3.54-3.50 (m, 6H), 2.93 (t, 1H, J=2.4 Hz), 2.31 (t, 2H, J=7.5 Hz), 1.93 (t, 2H, J=7.2 Hz), 1.60 (m, 2H), 1.39 (m, 2H), 1.25 (m, 2H), 1.12 (m, 2H).

### Example 3.1

### Preparation of N-phenyl-N′-[[1-[2-[2-[2-(2-prop-2-ynoxyethoxy)ethoxy]ethoxy]ethyl]triazol-4-yl]-bis(p-tolyl)methoxy]octanediamide 8e

same method as for 8d. white solid (26 mg, 0.04 mmol, 15%). TLC MeOH/DCM 5/95 Rf=0.2; ¹H NMR (400 MHz, DMSO-D₆) δ=10.16 (s, 1H), 9.83 (s, 1H), 7.78 (s, 1H), 7.57 (d, 2H, J=8.0 Hz), 7.25 (t, 2H, J=8.0 Hz), 7.10 (dd, 8H, J=8.0 Hz), 6.99 (t, 1H, J=8.0 Hz), 4.48 (t, 2H, J=4.0 Hz), 4.12 (s, 2H), 3.77 (t, 2H, J=4.0 Hz), 3.48 (m, 13H), 2.28 (s, 6H), 1.79 (t, 2H, J=8.0 Hz), 1.23 (t, 2H, J=8.0 Hz), 1.17 (m, 4H), 1.02 (q, 4H, J=8.0 Hz);

¹³C NMR (100 MHz, DMSO-D₆) δ=171.2, 170.0, 148.5, 139.3, 136.6, 128.6, 128.1, 128.0, 125.7, 122.8, 118.9, 86.8, 80.3, 77.1, 69.7, 69.6, 69.5, 69.4, 68.7, 68.5, 57.4, 49.2, 36.3, 32.1, 28.2, 24.7, 20.6; HRESI-MS: calcd. for [M+Na]⁺ (C₄₂H₅₃N₅O₇Na): 762.38372. found: 762.3837.

### Example 3.2

### Preparation of N¹-((1-(3,6,9,12-tetraoxapentadec-14-ynyl)-1H-1,2,3-triazol-4-yl)bis(4-fluorophenyl)methoxy)-N⁸-phenyloctanediamide (8g)

Add, at ambient temperature, 30.00 μl of acetyl chloride (0.42 mmol; 5 eq.) to a mixture of 42.00 mg of compound (7g) (0.08 mmol; 1 eq.) in 0.50 ml of toluene. Stir the mixture under reflux (100° C.) for 2 h.

Then, evaporate the toluene in the mixture under vacuum.

The intermediate chloride obtained is diluted in a minimum of distilled ACN and is added to a mixture of 44.00 mg of SAHA (0.17 mmol; 2 eq.) in 2 ml of distilled ACN. Then add 47.00 μl of distilled Et₃N (0.34 mmol; 4 eq.) and stir the mixture for 12 h at ambient temperature.

The unreacted SAHA is filtered with ACN. The mixture is evaporated under vacuum and then purified by combi flash using dry solvents (DCM/MeOH: from 0% to 5% in MeOH).

24.00 mg of product (8g) is obtained in the form of a yellow solid.

The starting alcohol (7g) is also recovered.

Yield: 38%

Rf (DCM/MeOH:95/5): 0.41

MP: 132.1° C.

¹H NMR (ACETONE D⁶, 400 MHz) δ (ppm): 1.25 (m, 6H); 1.61 (t, 2H, J=8 Hz); 1.94 (t, 2H, J=8 Hz); 2.31 (t, 2H, J=8 Hz); 2.93 (s, 1H); 3.52 (m, 12H); 3.89 (t, 2H, J=8 Hz); 4.15 (s, 2H); 4.57 (t, 2H, J=8 Hz); 7.02 (t, 3H, J=8 Hz); 7.11 (t, 3H, J=8 Hz); 7.45 (m, 4H); 7.65 (d, 2H, J=8 Hz); 7.79 (s, 1H); 9.08 (s, 1H); 9.74 (s, 1H).

¹³C NMR (ACETONE D⁶, 75.4 MHz) δ (ppm): 25.9; 33.3; 37.6; 50.8; 58.5; 69.7; 69.9; 70.9; 71.0; 71.1; 71.2; 71.3; 75.8; 80.8; 115.1; 115.3; 119.9; 123.8; 126.6; 129.4; 131.4; 131.5; 139.5; 140.6; 162.0; 171.9

¹⁹F NMR (ACETONE D⁶, 400 MHz) δ (ppm): −116.1

### Example 3.3

### Preparation of N¹-((1-(3,6,9,12-tetraoxapentadec-14-ynyl)-1H-1,2,3-triazol-4-yl)bis(4-chlororophenyl)methoxy)-N⁸-phenyloctanediamide (8h)

Same procedure as for the synthesis of compound (8g).

57.00 mg of compound (7h) (0.11 mmol; 1 eq.); 40.00 μl of acetyl chloride (0.53 mmol; 2 eq.); 0.60 ml of toluene; 56.50 mg of SAHA (0.21 mmol; 2 eq.); 60.00 μl of Et₃N (0.43 mmol; 4 eq.); 3.00 ml of ACN.

This gives 20 mg of product (8h) is obtained in the form of a yellow solid.

Yield: 24%

Rf (DCM/MeOH:95/5): 0.34

MP: 107.7° C.

¹H NMR (ACETONE D⁶, 400 MHz) δ (ppm): 1.24 (m, 6H); 1.57 (t, 2H, J=8 Hz); 1.92 (t, 2H, J=8 Hz); 2.30 (t, 2H, J=8 Hz); 2.93 (s, 1H); 3.53 (m, 12H); 3.87 (t, 2H, J=8 Hz); 4.15 (s, 2H); 4.56 (t, 2H, J=8 Hz); 7.00 (t, 1H, J=8 Hz); 7.25 (t, 3H, J=8 Hz); 7.45 (m, 6H); 7.64 (d, 2H, J=8 Hz); 7.83 (s, 1H); 9.08 (s, 1H); 9.75 (s, 1H).

¹³C NMR (ACETONE D⁶, 75.4 MHz) δ (ppm): 26.0; 37.6; 50.9; 58.6; 69.8; 70.0; 70.9; 71.0; 71.1; 71.2; 71.3; 75.8; 80.8; 119.9; 123.8; 128.7; 129.4; 129.8; 131.1; 134.3; 140.6; 171.9

### Example 4

### Preparation of N-(2-((1-(3,6,9,12-tetraoxapentadec-14-ynyl)-1H-1,2,3-triazol-4-yl)diphenylmethylamino)phenyl)-4-acetamidobenzamide (9c)

In this example, R₃ is an epigenetic modulator: CI-994

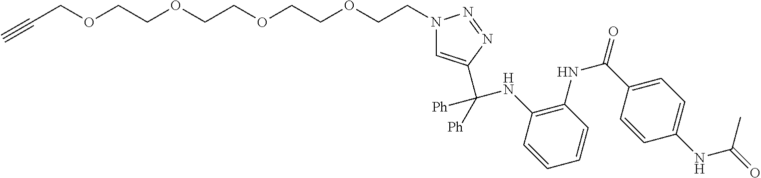 [SMILES: C#CCOCCOCCOCCOCCn1cc(C(Nc2ccccc2NC(=O)c2ccc(NC(C)=O)cc2)(c2ccccc2)c2ccccc2)nn1]

The following are added to 5 mL of dry DCM under a nitrogen atmosphere: 7c (0.20 g; 0.43 mmol; 1 eq.) and a 2M HCl solution in ether (0.43 mL; 0.85 mmol; 2 eq.). The solution is refluxed for 2 hours. The solution is then co-evaporated with toluene to give the intermediate chloride in the form of an oil. The preceding chloride dissolved in a few mL of ACN is added under nitrogen at ambient temperature to a solution of CI-994 (0.231 g; 0.90 mmol; 2 eq.) and dry Et₃N (0.24 mL; 1.72 mmol; 4 eq.) in 5 mL of dry CAN. The solution is stirred for 12 h at ambient temperature. The solvent is evaporated off under vacuum. The unreacted CI-994 is recovered by precipitation from an acetone solution and the residue is purified (flash chromatography, silica gel, eluent DCM/MeOH/Et₃N) to give the expected compound 9c (0.142 mg; 0.17 mmol, 39%). The unreacted alcohol 7c is recovered during the purification.

¹H-NMR (acetone d₆, 400 MHz): δ=9.44 (bs, 2H), 8.02 (d, 2H, J=8.6 Hz), 7.82 (s, 1H), 7.77 (d, 2H, J=8.7 Hz), 7.65 (m, 4H), 7.24 (m, 5H), 7.18 (m, 2H), 6.70 (dt, 1H, J=1.6 Hz, J=8.1 Hz), 6.61 (dt, 1H, J=1.3 Hz, J=7.5 Hz), 6.17 (dd, 1H, J=1.1 Hz, J=8.2 Hz), 6.12 (s, 1H), 4.50 (t, 2H, J=5.2 Hz), 4.14 (d, 2H, J=2.4 Hz), 3.79 (t, 2H, J=4.9 Hz), 3.59 (m, 2H), 3.55 (m, 2H), 3.48 (m, 4H), 3.43 (m, 4H), 2.92 (t, 1H, J=2.4 Hz), 2.12 (s, 3H).

¹³C-NMR (acetone-d₆, 100 MHz): δ=169.31, 152.91, 146.48, 143.54, 141.89, 129.90, 129.46, 128.94, 128.80, 127.69, 127.45, 126.82, 126.50, 125.92, 119.22, 118.61, 118.08, 80.95, 75.79, 71.21, 71.17, 70.97, 70.24, 69.80, 65.74, 58.58, 50.89, 24.41, 22.93, 15.20,

### Example 5

### Preparation of N-(2-((1-(3,6,9,12-tetraoxapentadec-14-ynyl)-1H-1,2,3-triazol-4-yl)bis(4-methoxyphenyl)methylamino)phenyl)-4-acetamidobenzamide (9d)

In this example, R₃ is an epigenetic modulator: CI-994

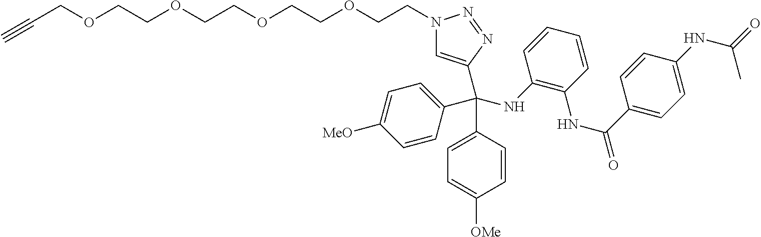 [SMILES: C#CCOCCOCCOCCOCCn1cc(C(Nc2ccccc2NC(=O)c2ccc(NC(C)=O)cc2)(c2ccc(OC)cc2)c2ccc(OC)cc2)nn1]

The following are added to 5 mL of dry DCM under a nitrogen atmosphere: 7d (0.20 g; 0.38 mmol; 1 eq.) and a 2M HCl solution in ether (0.38 mL; 0.76 mmol; 2 eq.). The solution, which has turned red, is stirred for 2 hours. The mixture is co-evaporated under vacuum to give the chloride in the form of a red oil. The preceding chloride dissolved in a few mL of ACN is added at ambient temperature to a solution of 5 mL of dry CAN containing CI-994 (0.205 g; 0.76 mmol; 2 eq.) and dry Et₃N (0.21 mL; 1.52 mmol; 4 eq.). The red colour disappears in a few minutes and the solution is stirred for 12 h at ambient temperature. After concentration under vacuum, the residue is taken up in acetone and the remaining CI-994 is precipitated. The solution obtained is concentrated and purified (automated flash chromatography, silica gel, eluent DCM/EtOH/Et₃N) to give 9d (0.087 g; 0.112 mmol; 29%) in the form of a pink oil. The unreacted alcohol 7d is also recovered.

Rf (DCM/EtOH/Et₃N 97:2:1): 0.24

¹H-NMR (acetone-d₆, 400 MHz) J: δ=9.47 (s, 1H), 9.42 (s, 1H), 8.02 (d, 2H), 7.78 (m, 3H), 7.51 (m, 4H), 7.24 (d, 1H), 6.79 (m, 4H), 6.73 (t, 1H), 6.62 (t, 1H), 6.19 (d, 1H), 5.99 (s, 1H), 4.49 (t, 2H), 4.14 (d, 2H), 3.80 (t, 2H), 3.73 (s, 6H), 3.59 (m, 2H), 3.55 (m, 2H), 3.48 (m, 4H), 3.43 (m, 4H), 2.92 (t, 1H), 2.12 (s, 3H).

¹³C-NMR (acetone-d₆, 100 MHz): δ=158.2, 152.4, 142.4, 140.9, 137.384, 129.0, 128.8, 128.3, 126.3, 125.6, 125.4, 124.5, 118.1, 117.6, 116.8, 116.6, 112.8, 79.8, 74.7, 70.1, 70.0, 69.8, 69.1, 68.7, 63.8, 57.5, 54.3, 49.7, 23.3,

### Example 6

### Preparation of N¹-((1-(3,6,9,12-tetraoxapentadec-14-ynyl)-1H-1,2,3-triazol-4-yl)bis(4-fluorophenyl)methoxy)phenyl (21g)

Add 50.30 μl of acetyl chloride (0.71 mmol; 5 eq.) to a mixture of 71.00 mg of compound (7g) (0.14 mmol; 1 eq.) in 0.70 ml of toluene. This mixture is to be refluxed for 2 h.

The toluene is evaporated off under vacuum.

The intermediate chloride is diluted in 5 ml of THF. 40.00 mg of phenol (0.43 mmol; 3 eq.) is added to this mixture, followed by 80.00 μl of Et3N (0.57 mmol; 4 eq.) and 5.00 mg of DMAP (0.04 mmol; 0.3 eq.). Reflux the mixture (63° C.) with stirring for 12 h.

Add 20 ml of DCM to the mixture. The organic phase is washed with 3×10 ml of H₂O, dried, filtered and then evaporated under vacuum.

The mixture is purified by combi flash (using dry solvents): DCM/MeOH from 0% to 4% in methanol.

28 mg of product (10g) is obtained in the form of a clear oil.

Yield: 34%

Rf (DCM/MeOH:95/5): 0.46

¹H NMR (ACETONE D⁶, 400 MHz) δ (ppm): 2.92 (s, 1H); 3.45 (m, 12H); 3.79 (t, 2H, J=8 Hz); 4.15 (s, 2H); 4.52 (t, 2H, J=8 Hz); 6.80 (m, 3H); 7.05 (m, 6H); 7.70 (m, 4H); 7.75 (s, 1H).

¹³C NMR (ACETONE D⁶, 75.4 MHz) δ (ppm): 50.8; 58.5; 69.7; 70.0; 70.9; 71.0; 71.1; 75.7; 80.8; 83.8; 115.3; 115.4; 120.8; 122.3; 126.4; 129.41; 130.3; 141.6; 150.2; 156.2; 161.4; 163.8

¹⁹F NMR (ACETONE D⁶, 400 MHz) δ (ppm): −116.8

C) Preparation of the Compounds of Formula I in which R1 Represents a Group of Formula (III)

### Example 6

### Preparation of α-norbornenyl poly(ethylene oxide) nitride NB-PEO-N₃ (13) (Diagram II)

### Throughout the Description, NB-PEO Means α-Norbornenyl Poly(Ethylene Oxide)

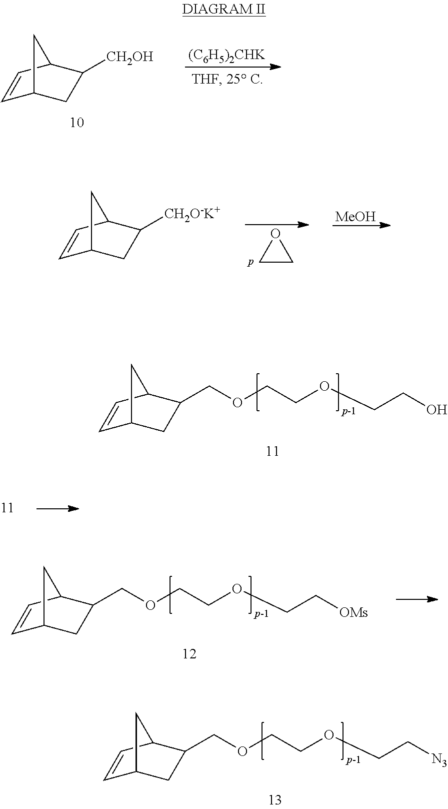

### 6.1: Preparation of NB-PEO (11)

The macromonomer α-norbornenyl poly(ethylene oxide) (NB-PEO) 11 was obtained by ring-opening metathesis polymerization of ethylene oxide according to the experimental protocols described in the literature (Heroguez, V.; Breunig, S.; Gnanou, Y.; Fontanille, M., Macromolecules 1996, 29, (13), 4459-4464).

Norbonene methanol 10 (0.79 mL, 6.6 mmol), deprotonated with diphenylmethyl potassium (9.5 mL, 0.61 mol.L⁻¹), is used as initiator for polymerizing 0.37 mol of ethylene oxide (18.5 mL). After 48 h, polymerization is deactivated with 3 mL of acidified methanol. The NB-PEO 11 is precipitated from diethyl ether and dried under vacuum (yield 91%).

¹H-NMR (ppm, CDCl₃, 400 MHz), (relative integral): δ=3.6 (262H, m), 4.3 (2H, m), 5.9-6.1 (2H, m).

### 6.2: Preparation of NB-PEO-OMs (12)

3.68 g of lyophilized NB-PEO 11 (1.22 mmol) is dissolved in 40 mL of anhydrous THF and then 4 equivalents of TEA (0.68 mL, 4.9 mmol) are added. 3.5 equivalents of methanesulphonyl chloride (0.33 mL, 4.3 mmol) are added under a nitrogen stream. The solution is stirred at ambient temperature overnight and filtered on a frit. The mesylated macromonomer α-norbornenyl poly(ethylene oxide) NB-PEO-OMs 12 obtained is precipitated from 200 mL of diethyl ether, filtered on a frit and dried under vacuum (yield 82%).

¹H-NMR (ppm, CDCl₃, 400 MHz), (relative integral): δ=3.0-3.1 (3H, s), 3.6 (262H, m), 4.3 (2H, m), 5.9-6.1 (2H, m).

### 6.3 Preparation of NB-PEO-N₃ (13)

0.64 g of NB-PEO-OMs 12 (0.21 mmol) and 70 equivalents of sodium nitride (0.95 g, 14.6 mmol) are dissolved in 20 mL of DMF and then stirred at ambient temperature for 40 h. 120 mL of dichloromethane is then added and the solution is washed five times with water (5×60 mL). The organic phase is dried over Na₂SO₄ and filtered on a frit. After evaporation of the solvent in a rotary evaporator, the residue is dissolved in 25 mL of THF, precipitated from 150 mL of diethyl ether and dried under vacuum (yield 86%).

¹H-NMR (ppm, CDCl₃, 400 MHz), (relative integral): δ=3.3 (2H, t), 3.6 (262H, m), 5.9-6.1 (2H, m).

The Compounds of Formula I in which R₁ Represents a Group of Formula (III) are Prepared According to Diagram III

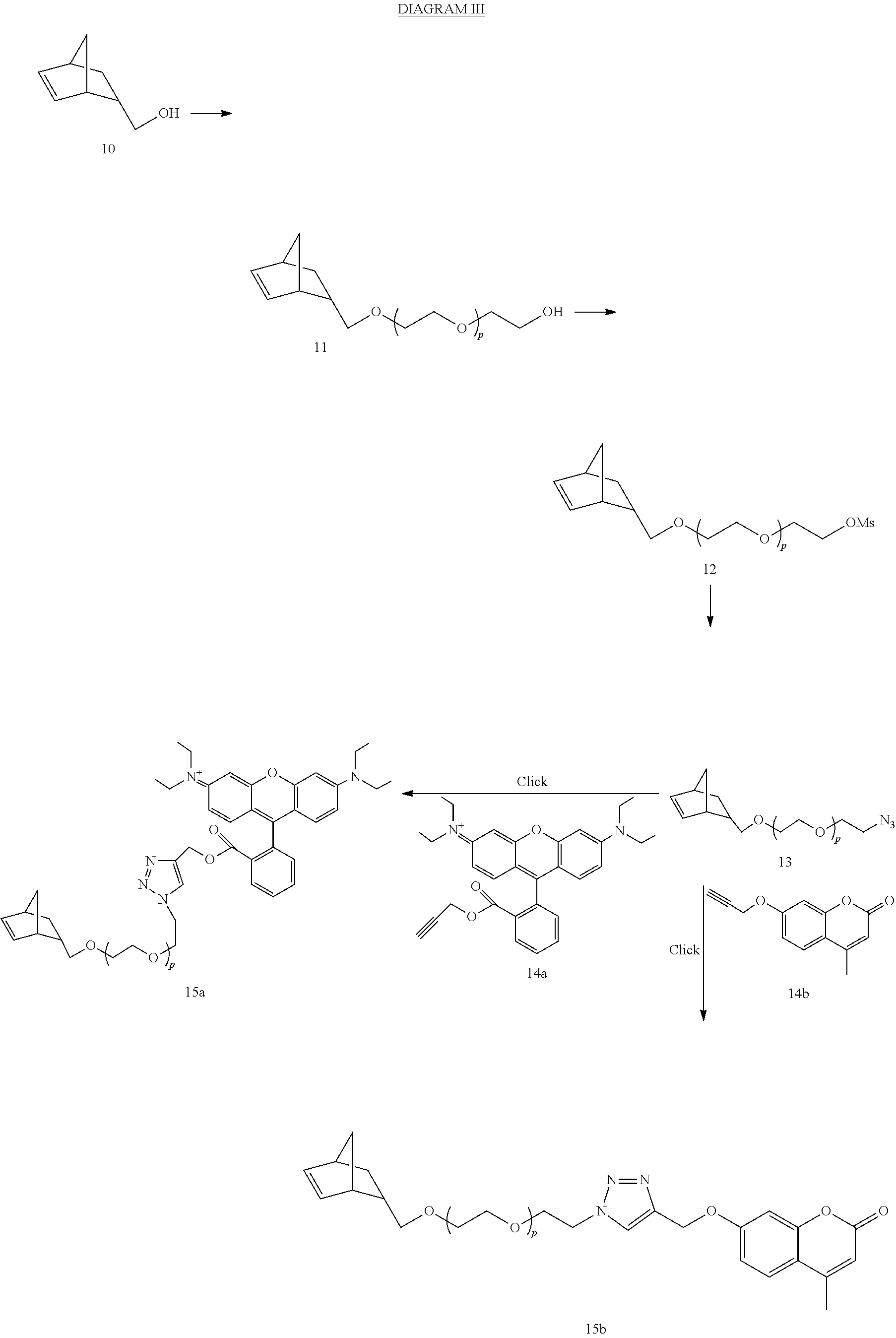

### Example 7

### Preparation of NB-PEO—Rhodamine B (15a)

In this example, R₃ is a detecting probe: Rhodamine B

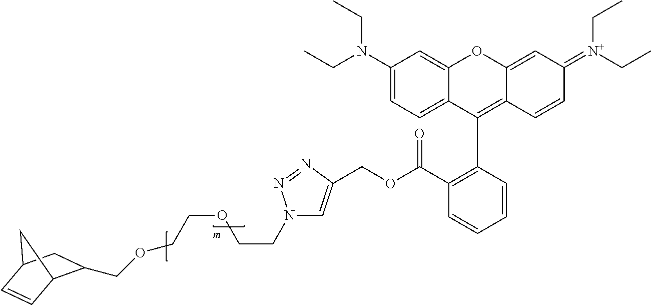

1 equivalent of NB-PEO-N_{3 1}3 (333.4 mg, 0.110 mmol), 1.5 equivalents of Rhodamine B alkyne 14a (85 mg, 0.165 mmol) and 2 equivalents of PMDETA (46 μL, 0.286 mmol) are dissolved in 10 mL of a degassed solution of dichloromethane/ethanol (35/65% V/V) and placed under an inert atmosphere. 2 equivalents of CuBr are added to the solution. The solution is stirred at ambient temperature for 6 days. After 6 days, the solvent is evaporated off and the polymer is dissolved in 10 mL of dichloromethane. The organic phase is washed 10 times with water and then dried over Na₂SO₄. The dichloromethane is evaporated off and the residue is dissolved in 15 mL of THF, precipitated from 100 mL of diethyl ether and then dried under vacuum to give the product 15a.

### Example 8

### Preparation of NB-PEO-Coumarin (15b)

In this example, R₃ is a detecting probe: Coumarin

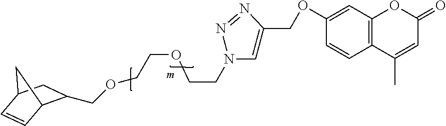

1 equivalent of NB-PEO-N₃ 13 (61.5 mg, 0.143 mmol), 2 equivalents of coumarin alkyne 14b (61 mg, 0.286 mmol) and 2 equivalents of PMDETA (0.06 mL, 0.286 mmol) are dissolved in 10 mL of a degassed solution of dichloromethane/ethanol (35/65% V/V) and placed under an inert atmosphere. 2 equivalents of CuBr are added to the solution. The solution is stirred at ambient temperature for 6 days. After 6 days, the solvent is evaporated off and the polymer is dissolved in 20 mL of dichloromethane. The organic phase is washed 10 times with water and then dried over Na₂SO₄. The dichloromethane is evaporated off and the residue is dissolved in 15 mL of THF, precipitated from 100 mL of diethyl ether and then dried under vacuum to give compound 15b.

### Example 9

### Preparation of NB-PEO-CI-994 (17c)

In this example, R₃ is an epigenetic modulator: CI-994

1 equivalent of NB-PEO-N_{3 1}3 (523 mg, 0.133 mmol), 1.5 equivalents of 9c (Example 4) (140 mg, 0.195 mmol) and 2 equivalents of PMDETA (54 μL, 0.26 mmol) are dissolved in 5 mL of degassed DMF and placed under an inert atmosphere. 2 equivalents of CuBr are added to the solution. The solution is stirred at ambient temperature for 4 days. After 4 days, the solvent is evaporated off and the polymer is dissolved in 30 mL of dichloromethane. The organic phase is washed 10 times with water and then dried over Na₂SO₄. The dichloromethane is evaporated off and the polymer is dissolved in 20 mL of THF, precipitated from 100 mL of diethyl ether and then dried under vacuum.

### Example 10

### Preparation of NB-PEO-CI-994 (17d)

In this example, R₃ is an epigenetic modulator: CI-994

This compound is synthesized in the same way as in Example 9 by reacting compound 13 with compound 9d described above

### Example 11

### Preparation of NB-PEO-SAHA (18c)

In this example, R₃ is an epigenetic modulator: SAHA

This compound is synthesized in the same way as in Example 9.

### Example 12a

### Preparation of NB-PEO-SAHA (18d)

In this example, R₃ is an epigenetic modulator: SAHA

This compound is synthesized in the same way as in Example 9.

### Example 12b

### Preparation of NB-PEO-SAHA (18e)

In this example, R₃ is an epigenetic modulator: SAHA

This compound is synthesized in the same way as in Example 9.

D) Preparation of Compounds of Formula I in which R₁ Represents a Group of Formula (II): Compounds of Formula VII or Functional Particles with Detecting Probe (Rhodamine B 16a, Coumarin 16b) or Epigenetic Modulator (CI-994 19c and 19d, SAHA 20c, 20d, 20e)

### Example 13

### Preparation of the Compounds of Formula VII

The copolymerization of NB with NB-PEO-N₃ is carried out at ambient temperature in a 100-mL flask, with stirring and an inert atmosphere. Typically, 7 mg (8.5 10⁻⁶ mol) of the first-generation Grubbs catalyst is dissolved in 3.6 mL of a degassed solution of dichloromethane/ethanol (50/50% V/V). The NB (0.28 g, 3 10⁻³ mol) and NB-PEO-N₃ (13) (0.15 g, 3027 g.mol⁻¹, 5.1 10⁻⁵ mol) are dissolved in 5 mL of a degassed solution of dichloromethane/ethanol (35/65% V/V) and added, under argon, to the solution of catalyst. Deactivation of the reaction mixture is carried out by adding 0.1 mL of ethyl vinyl ether.

The compounds of formula VII are thus obtained.

Colloidal characterization is obtained by dynamic light scattering and by imaging (transmission electron microscopy). Chemical characterization is obtained by nuclear magnetic resonance.

### Example 14

### Preparation of Compounds 16a and 16b

The compounds are prepared as in example 13 according to the following Diagram IV:

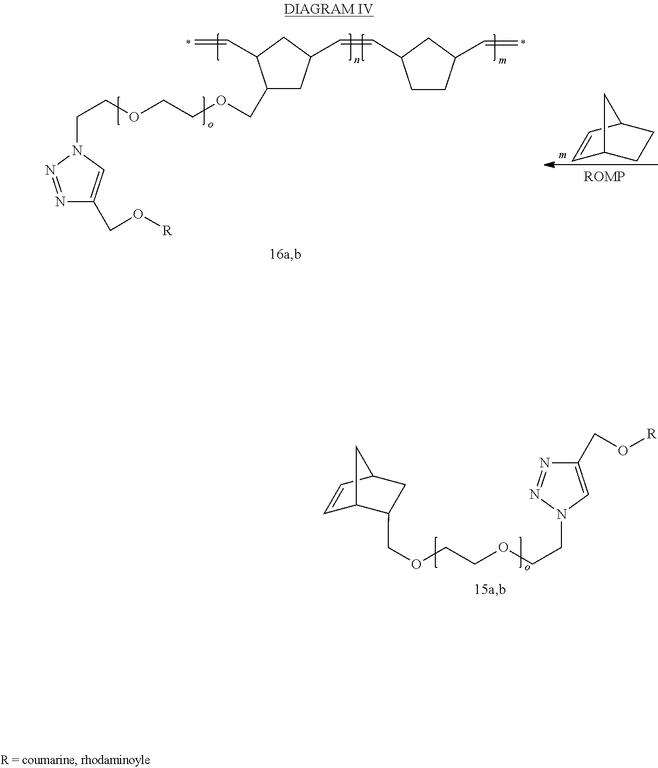

The compounds 16a and 16b can also be prepared by the following procedure:

0.85 equivalents (relative to the nitride function) of Rhodamine B alkyne (28 mg, 0.13 mmol) and 2 equivalents of PMDETA (6 μL, 0.03 mmol) are dissolved in 1 mL of degassed solution of dichloromethane/ethanol (35/65% V/V) and then placed under an inert atmosphere. 2 equivalents of CuBr are added to the solution. The solution is added to a dispersion of nitride nanoparticles synthesized beforehand (Example 13). The dispersion is stirred at ambient temperature for 4 days in order to obtain compound 16a.

By way of example, the following compound 16a is prepared according to the following Diagram IV-1:

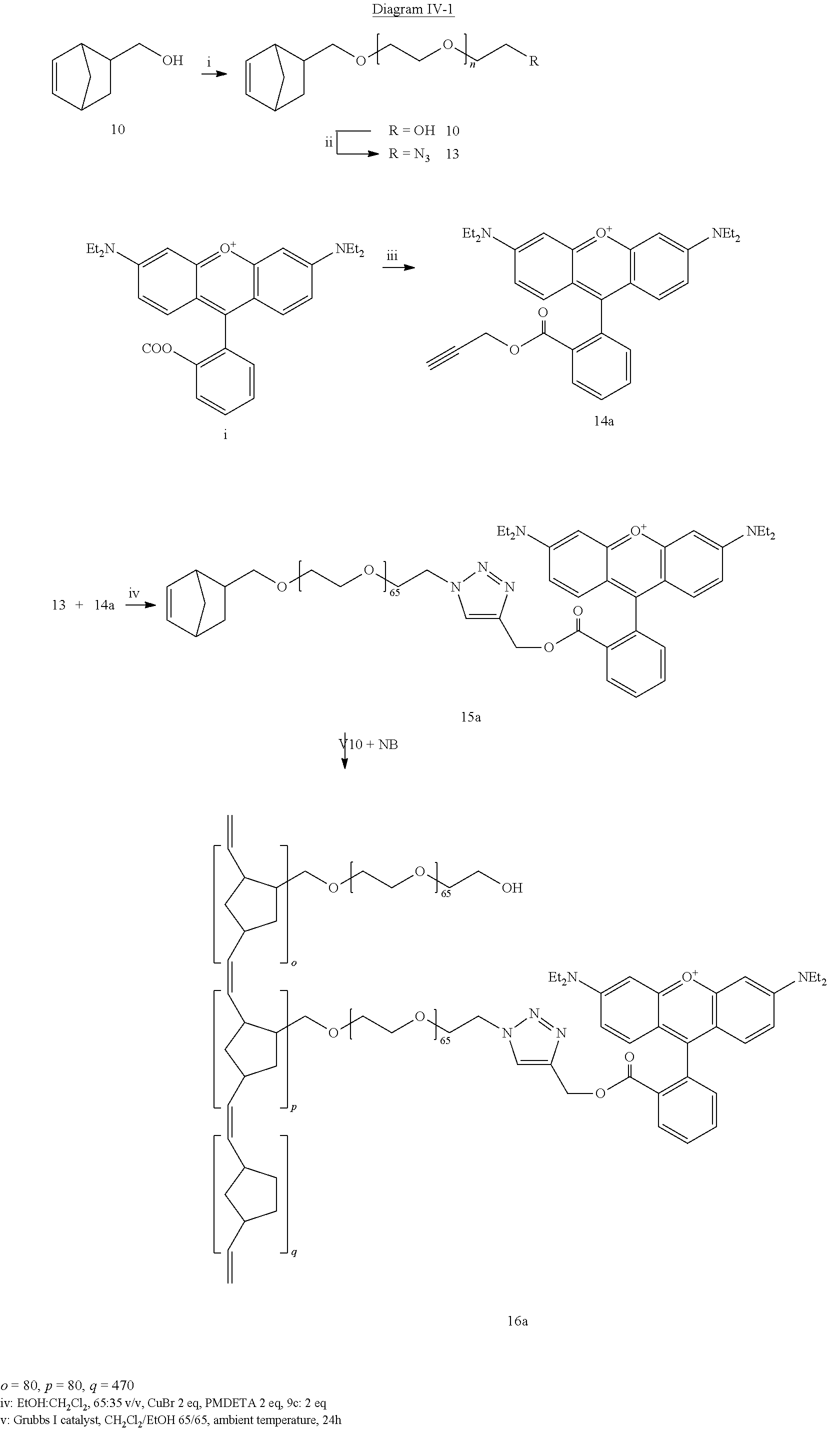

By way of example, the following compound 16b is prepared by the same method:

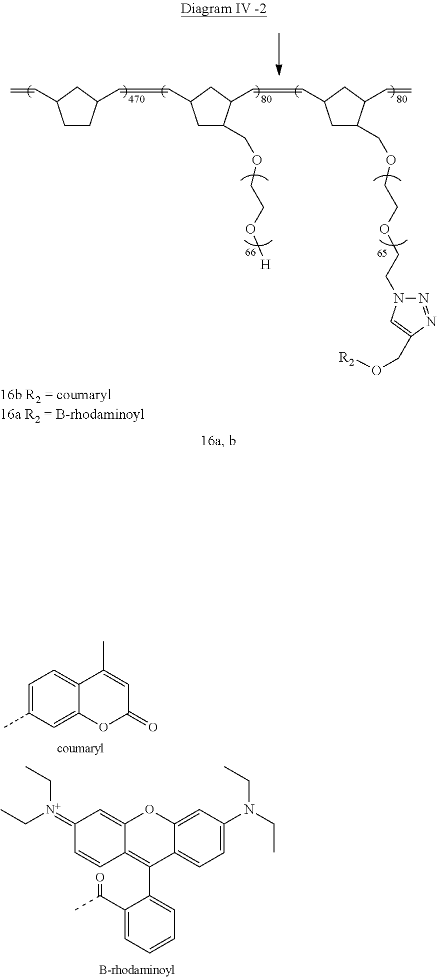

### Example 15

### Preparation of the Compounds 19c, 19d, 20c, 20d and 20e

The compounds are prepared as in Example 13 according to the following Diagram V:

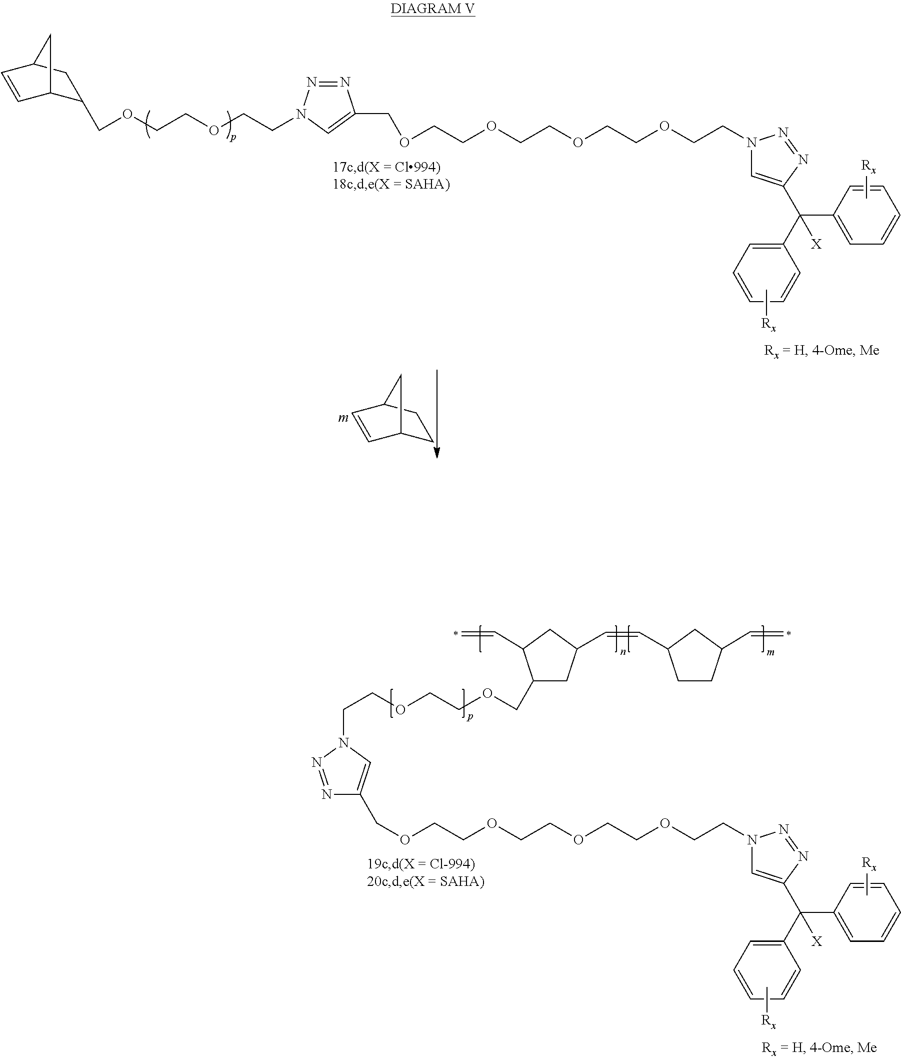

The compounds 19c,d and 20c,d,e are obtained in the same way starting from NB-PEO-CI-994 (17c,d) or NB-PEO-SAHA (18c,d,e) respectively.

Colloidal characterization is obtained by dynamic light scattering and by imaging (transmission electron microscopy). Chemical characterization is obtained by nuclear magnetic resonance.

By way of example, the following compound 19c is prepared according to Diagram VI:

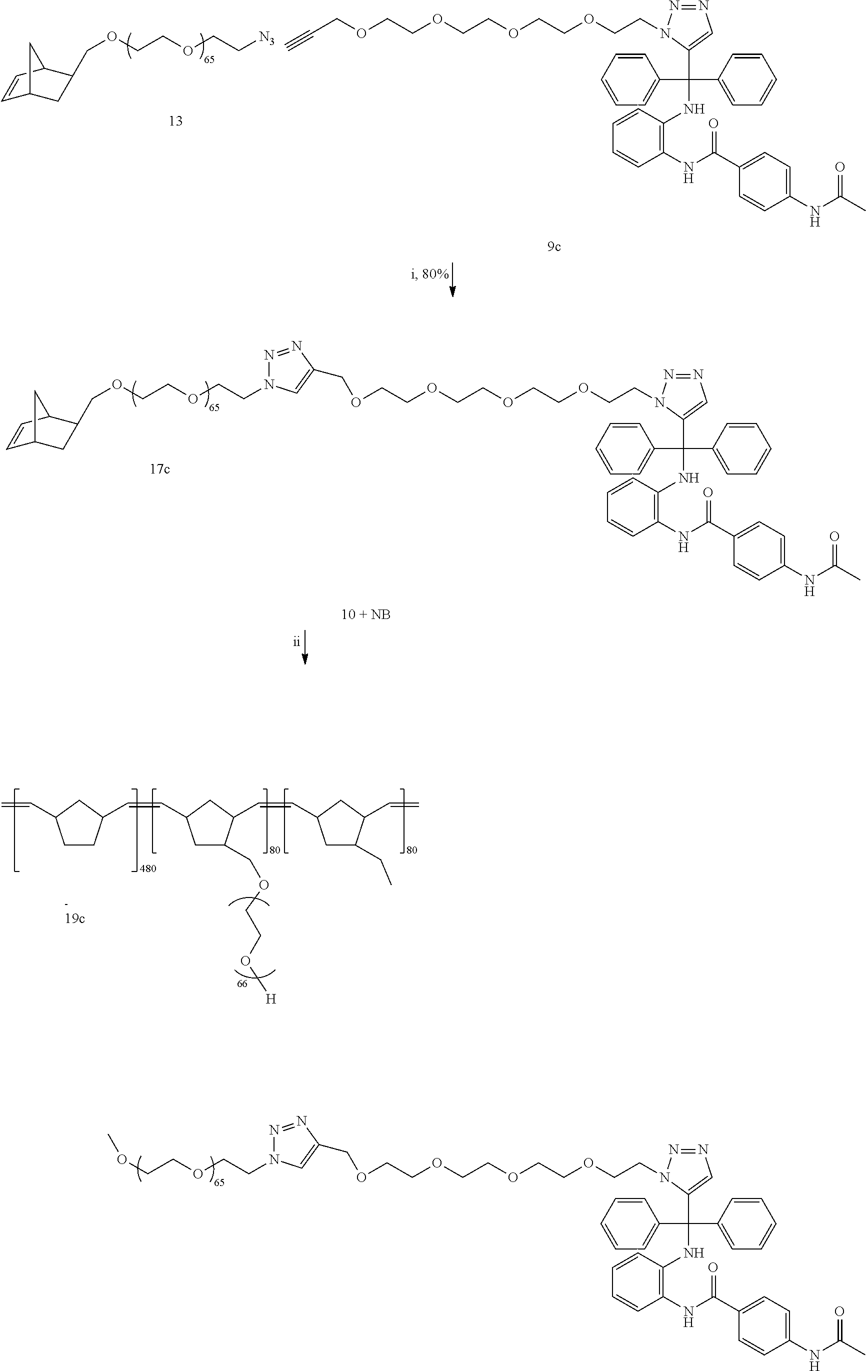

### Biological Section:

### Example 16

### Tests of the of Compound 9d of the Invention at pH 4.3, 5.3 and 7.3 (Physiological)

The compound is put into an acid medium (citrate buffer for pH 4.3 and Trisma buffer for pH 7.3) and release of CI-994 is monitored by HPLC.

The results are presented in FIGS. 3A and 3B and show that the compounds of the invention are hydrolysed rapidly at pH 4.3 and 5.3 (FIG. 3) and much more slowly at physiological pH.

### Example 16.1

The hydrolysis of the compounds of the invention was carried out in buffer solutions (citrate buffer for pH 4.3 and Trisma for pH 7.3) at pH=4.3 and at pH=7.3.

The concentration of the samples is 1 mg/ml in the solution: acetonitrile at 80% and buffer solution at 20%.

The different results are presented below.

| | t_{1/2} pH 4.3 | t_{1/2} pH 7.3 |
|---|---|---|
| (8e) | 20 h | 4 days |
| (9c) | 30 min | 3.6 days |
| (21g) | 24 days | stable |

The term stable defines a compound that does not show significant hydrolysis after several days, in particular more than four days. In some cases the measurements were prolonged for 2 or 3 weeks, or even a month.

The result obtained with compound (8e) is particularly interesting since the compound SAHA alone has a half-life of less than an hour. The particles of the invention, in particular comprising SAHA, therefore have a much longer half-life at physiological pH, permitting an improvement for the administration of this molecule.

In the case of product 21g, the term stable means that the product does not undergo hydrolysis for at least 1 month.

### Example 17

### Cellular Viability in the Presence of the Compounds of the Invention

### 17.1 Cell Culture

### Biological Material:

Human cell lines of mesotheliomas (Meso 4, Meso 13, Meso 34, Meso 56, Meso 76 and Meso 95B) and of lung adenocarcinomas (ADCA; ADCA 3, ADCA 72, ADCA 117 and ADCA 153) established at the Inserm U892 laboratory from pleural fluid of patients undergoing puncture at the Nantes CHU (Laënnec Hospital, St-Herblain).

### Culture Conditions:

Adherent cells growing in a monolayer on plastic substrate (flasks) in a sterile complete culture medium with the following composition:

RPMI 1640 (Gibco)

+10% Fetal calf serum treated beforehand by thermal shock (30 minutes at 56° C.)
+glutamine (2 mM)
+penicillin (100 IU/mL)/streptomycin (100 μg/mL)

The cells are kept in an incubator at 37° C. (5% CO₂) under a humid atmosphere.

### Passaging of the Cells (80% Confluence):

Rinsing the cells of RPMI 1640 alone

Incubation at 37° C. for 3-4 min with trypsin/EDTA (2 mL)

Neutralization of the trypsin by adding 10 mL of complete medium

Counting the cells on a Malassez plate

Seeding at the desired density for the different applications.

### 17.2 Cellular Viability Experiments

D0: In the morning, aspirate the culture medium. Wash with 2 ml of PBS.

Add 2.5 ml of trypsin/EDTA. Leave for 3-4 minutes at 37° C.

Add 10 ml of RPMI 10% FCS penicillin (100 U/ml), streptomycin (100 μg/ml) and L-glutamine (2 mM).

Dissociate the cells by aspiration-ejection.

Count the cells in a Malassez cell.

Seed 5000 cells per well of a 96-well plate in 180 μl of RPMI 10% FCS penicillin/streptomycin and glutamine.

D1: Add 10 μl of Uptiblue (Interchim). Leave for 2 h at 37° C.

Read on the Typhoon (GE Healthcare).

Prepare the cell treatment media: test compounds in RPMI 10% FCS penicillin/streptomycin and glutamine.

Aspirate the medium and then add the treatment media (180 μl).

D4: Wash twice with 180 μl of RPMI 10% FCS penicillin/streptomycin and glutamine.

Add 10 μl of Uptiblue. Leave for 2 h at 37° C.

Read on the Typhoon.

### Use of the Typhoon:

Acquisition mode: Fluorescence

Set-up −580 BP Filter and PMT 350

Definition: 200 μM

Focal plane: +3 mm

These experiments make it possible to evaluate the toxicity of the various compounds forming the functionalized nanoparticles as well as the release of the HDAC inhibitors (1HD) in the cells by measuring the cellular viability. The results are in particular a decrease in cellular viability in the presence of free iHDACs. These experiments also make it possible to define the possible range of toxicity of the nanoparticles.

### Example 18

### Internalization of the Compounds of the Invention

### 18.1: BRET

Cellular model: Met-5A cells (ATCC)

D0: In the morning, aspirate the culture medium of the Met-5A cells. Wash with 2 ml of PBS.

Add 2.5 ml of trypsin/EDTA. Leave for 3-4 minutes at 37° C.

Add 10 ml of RPMI 10% FCS penicillin (100 U/ml), streptomycin (100 μg/ml) and L-glutamine (2 mM).

Dissociate the cells by aspiration-ejection.

Count the cells in a Malassez cell.

Seed 200000 cells per well of a 6-well plate in 2 ml of RPMI 10% FCS penicillin/streptomycin and glutamine.

D1: Transfection: phRluc-C₁ BrD+pEYFP-C₁ histone H3

For one well of a 6-well plate in a 1.5 ml Eppendorf:

600 ng of phRluc-C₁ BrD+1500 ng of pEYFP-C₁ histone H3 in 100 μl of crude RPMI.

600 ng of phRluc-C₁ BrD in 100 μl of crude RPMI.

Add 3 μl of Attracten (Qiagen) directly in the liquid. Homogenize by tapping the tube with a finger.

Leave for 20 minutes at ambient temperature. During this time, change the medium of the cells seeded on D0 with 2 ml of RPMI 10% FCS penicillin/streptomycin and glutamine.

Add the DNA/Attracten mixture (100 μl) to the cells.

Leave for 24 h at 37° C. under 5% CO₂ and in a humid atmosphere.

D2: Wash the well with 1 ml of PBS and then detach the cells with 300 μl of trypsin/EDTA.

Leave for 3-4 minutes at 37° C.

Add 2 ml of RPMI 10% FCS penicillin (100 U/ml), streptomycin (100 μg/ml) and L-glutamine (2 mM).

Dissociate the cells by aspiration-ejection.

Distribute 180 μl of cellular suspension in the wells of a white 96-well plate (Optiplate, Berthold).

After 6 h at 37° C., begin the treatments by adding 20 μl of treatment medium (RPMI 10% FCS penicillin, streptomycin and L-glutamine) comprising the test compounds at a ten-fold concentration.

D3: Remove the cells from the incubator and wash the cells with 45 μl of PBS at ambient temperature.

Aspirate the PBS and then add 45 μl of PBS.

Add 5 μl per well of coelenterazine h (Interchim) at 25 μM.

Wait 10 minutes at ambient temperature.

Read the plate on the Mithras (Berthold)

Software: Microwin 2000

Program: BRET

Parameters: Reading at 485 nm for 1 second and then reading at 530 nm for 1 second.

The plate is read 5 times.

The 5 values obtained for one well are averaged.

Calculation of the BRET:

((value 530 nm phRluc-C₁ BrD+pEYFP-C₁ histone H3/value 480 nm phRluc-C₁ BrD+pEYFP-C₁ histone H3)-(value 530 nm phRluc-C₁ BrD/value 480 nm phRluc-C₁ BrD))×1000

Unit: milli BRET unit (mBu)

These experiments make it possible to evaluate the release of the HDAC inhibitors (HDI) in the live cells. The results are an increase in the BRET signal in the presence of free HDI.

### 18.2: Fluorescence Microscopy: Internalization of the Nanoparticles

D0: Put coverglasses in the wells of a 12-well plate.

Incubate the coverglasses in 100% ethanol (2 mL/well) for 1 h, remove and leave to evaporate under a hood (about 15 to 30 min).

Rinse once with PBS: 1 mL of PBS per well.

Put the coverglasses to dry under the hood (from 15 to 30 min).

Aspirate the culture medium from the cells. Wash with 2 ml of PBS.

Add 2.5 ml of trypsin/EDTA. Leave for 3-4 minutes at 37° C.

Add 10 ml of RPMI 10% FCS penicillin (100 U/ml), streptomycin (100 μg/ml) and L-glutamine (2 mM).

Dissociate the cells by aspiration-ejection.

Count the cells in a Malassez cell.

Seed 50000 cells per coverglass in 1 ml of RPMI 10% FCS penicillin/streptomycin and glutamine.

D1: Incubate the cells with 21.5 μg/ml of rhodamine B nanoparticles in 1 ml of culture medium for 2.5 h under the cell culture conditions.

The cells are washed with 1 ml of PBS.

The cell membranes are labelled with PKH67 (Sigma) according to the manufacturer's instructions.

The cells are fixed with 4% paraformaldehyde (Sigma) in the culture medium for 15 to 20 minutes at ambient temperature containing 1 μg/mL Hoechst (Sigma).

Rinsing with 1 ml of PBS per well, 3 times.

Rinse the coverglasses in water then mount in the mounting solution.

### Mounting the Coverglasses:

Put 5 μL of ProLong® Gold (Molecular Probes) per coverglass on the slide.

Using a needle and tweezers, remove the coverglass from the well, and immerse it briefly in sterile water.

Pay attention to the direction of depositing the coverglass, so that the cells are between the slide and the coverglass.

Fixation: overnight in darkness.

These experiments must make it possible to observe the presence of red dots (nanoparticles coupled to rhodamine B) inside a green border (plasma membrane). This will confirm internalization of the nanoparticles by the cells.

### 18.3: Fluorescence Microscopy: Co-Localization of Nanoparticles and Acidic Intracellular Compartments

D0: Put coverglasses in the wells of a 12-well plate.

Incubate the coverglasses in 100% ethanol (2 mL/well) for 1 h, remove and leave to evaporate under a hood (about 15 to 30 min).

Rinse once with PBS: 1 mL of PBS per well.

Put the coverglasses under a hood to dry (from 15 to 30 min).

Aspirate the culture medium from the cells. Wash with 2 ml of PBS.

Add 2.5 ml of trypsin/EDTA. Leave for 3-4 minutes at 37° C.

Add 10 ml of RPMI 10% FCS penicillin (100 U/ml), streptomycin (100 μg/ml) and L-glutamine (2 mM).

Dissociate the cells by aspiration-ejection.

Count the cells in a Malassez cell.

Seed 50000 cells per coverglass in 1 ml of RPMI 10% FCS penicillin/streptomycin and glutamine.

D1: Incubate the cells with 21.5 μg/ml of rhodamine B nanoparticles in 1 ml of culture medium for 2.5 h under the cell culture conditions.

The cells are washed with 1 ml of PBS.

The cell membranes are labelled with PKH67 (Sigma) according to the manufacturer's instructions.

The cells are fixed with 4% paraformaldehyde in the culture medium for 15 to 20 minutes at ambient temperature protected from the light (125 μL of the parent solution at 32% (Sigma) in 1 ml of culture medium) containing 1 μg/mL Hoechst (Sigma).

Rinsing with 1 ml of PBS per well, 3 times.

Permeabilization for 5 minutes at ambient temperature: 800 μL per well of PBS 1X Triton X100 0.05% Tween 0.05%.

Rinsing with 1 mL of PBS per well, 3 times (1 quick and 2 of 5 min)

Saturation for 10 to 20 min in 800 μL of PBS+BSA 1% (without rinsing)

Incubation with the anti-LamP1 primary antibody (1 μg/ml) diluted in 600 μL of PBS for 90 min at ambient temperature.

Rinsing with 1 mL of PBS per well, 3 times (1 quick and 2 of 5 min)

Incubation with the fluorescent secondary antibody (Cy5 mouse (Jackson) at 1/200) diluted in 600 μL of PBS⁺ for 60 min at ambient temperature and in darkness.

Rinsing with 1 mL of PBS per well, 3 times (1 quick and 2 of 5 min)

Rinse the coverglasses in water, then mount in the mounting solution.

### Mounting the Coverglasses:

Put 5 μL of ProLong® Gold (Molecular Probes) per coverglass on the slide.

Using a needle and tweezers, remove the coverglass from the well, and immerse it briefly in sterile water.

Pay attention to the direction of depositing the coverglass so that the cells are between the slide and the coverglass.

Fixation: overnight in darkness.

FIGS. 5A to 5C present the results obtained with the particles 16a of the invention and show that the particles detected in the cell co-localize with the lysosomal vesicles and that consequently they have been internalized by endocytosis.

### 18.4: Flow Cytometry (FACS)

D0: Seed the cells in a 6-well plate at a density of 200000 cells/well in RPMI medium 10% FCS penicillin (100 U/ml), streptomycin (100 μg/ml) and L-glutamine (2 mM).

D1: Incubate the cells with the nanoparticles in suspension in RPMI medium 10% FCS penicillin (100 U/ml), streptomycin (100 μg/ml) and L-glutamine (2 mM). (3.45 mg/mL) at 37° C. or on ice for 2.5 h.

Recover the cells from the supernatant and the adherent cells by trypsinization.

Obtain a cell deposit by centrifugation and wash twice with cold PBS to remove any trace of nanoparticles in suspension.

Transfer the cells to small FACS tubes for analysis by flow cytometry

(FACSCalibur cytometer/software CellQuest Pro, BD Biosciences)

Measure cell size and granulometry using FSC (“Forward SCatter”) and SSC (“Side SCatter”) channels.

Analyse the data obtained using the FlowJo software (Tree Star).

### Example 19

### Biodistribution of the Nanovectors

Female nude mice are injected with 3 million of AK7 (100 μl) subcutaneously in the left flank.

3 weeks later, 0.02 mg/g of mouse (100 μl) of a solution of nanovectors labelled with rhodamine B is injected into one of the veins of the tail previously dilated in hot water.

During this step, the mice are immobilized.

The animals are euthanized by cervical dislocation.

The biodistribution of the nanovectors is observed by fluorescence imaging between 1 h and 1 week (Biospace Lab, Excitation: 580 nm and Emission: 630 nm).

The images are analysed with the Photo vision+1.3 software (Biospace Lab).

The results are shown in FIG. 6.

FIG. 6 shows that 24 h after intravenous injection of the nanovectors (compounds 16a, Diagram IV-1 of Example 14), there is very pronounced localization at the level of the tumour and in much lower quantity in the liver.

Kinetic studies of biodistribution show a high initial plasma concentration, which decreases rapidly after 1 hour. The same observation can be made in the kidneys but with a smaller quantity of compound 16a and an even smaller quantity in the ovaries.

The level detected in the spleen and the brain is not significant compared with the control.

A larger quantity is detected in the liver but the main result is the very high selectivity for the tumour.

The rapid decrease in the blood compared with the progressive accumulation in the tumours can be explained by the whole body distribution at low levels in the mice, almost all of compound 16a finally being accumulated in the tumours.

These results show that compound 16a is not eliminated by the spleen, the kidneys or the liver, which represent the main biological barriers to dissemination of the nanoparticles.

The short half-life of the compounds of the invention in the blood (less than 6 h) is correlated with their rapid accumulation in the tumour. This observation thus validates the compatibility of the nanovectors with targeting of the tumour by the EPR effect and suggests that the structural properties of the compounds of the invention allow highly effective dissemination of the tumour by the EPR effect.

1. Nanovectors constituted by polymer chains Pi of the following general formula (I): 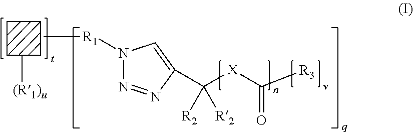
in which: 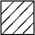
represents a polymer chain P, in particular a polymer chain P containing about 30 to 10,000 monomer units, identical or different, derived from the polymerization of monocyclic alkenes in which the number of carbon atoms constituting the ring is from about 4 to 12, or of polycyclic alkenes in which the total number of carbon atoms constituting the rings is from about 6 to 20,
t represents 0 or 1,
q is an integer in the range from 1 to 10,
u is an integer in the range from 0 to 10,
n represents 0 or 1,
v represents 0 or 1,
X represents O, NH or S,
R₁ and R′ₗ represent, independently of one another, when t=1, a group of the following Formula (II): 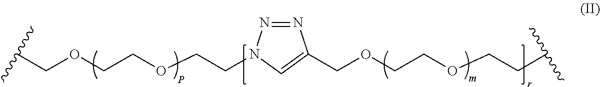
where:
m and p represent, independently of one another, an integer from 1 to 1000, in particular 50 to 340, particularly 70 to 200
r represents an integer in the range from 0 to 10, preferably 0 or 1,
or,
R₁ represents, when t=0, a group of the following Formula (III) linked to a monocyclic alkene or a polycyclic alkene: 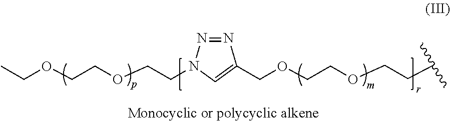
in which the number of carbon atoms constituting the ring of the monocyclic alkene is from about 4 to 12, and the total number of carbon atoms constituting the rings of the polycyclic alkene is from about 6 to 20,
r, m and p being as defined above,
or,
R₁ represents, when t=0, a group of the following Formula (IV): 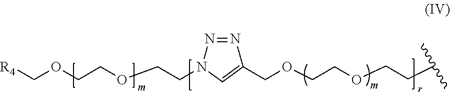
in which R₄ represents: a vinyl group, an ethyne group, an OR′ or SR″ group, R′ and R″ representing, independently of one another, H, a C₁-C₂₀ alkyl, a C₃-C₂₀ cycloalkyl, and m being as defined above,
r represents an integer in the range from 0 to 10, preferably 0,
R₂ and R′₂ represent, independently of one another:
H or a phenyl, unsubstituted or substituted by at least:
a C₁-C₂₀ alkyl, a C₃-C₂₀ cycloalkyl,
a C₁-C₂₀ alkoxy,
NRₐR_{b} where Rₐ and R_{b} represent, independently of one another, H, a C₁-C₂₀ alkyl, the alkyl being able to form a ring with the carbon ortho to that bearing N, a C₃-C₂₀ cycloalkyl,
NO₂,
CO₂Rc, where Rc represents H, a C₁-C₂₀ alkyl, a C₃-C₂₀ cycloalkyl, a substituted or unsubstituted benzyl,
a C₂-C₂₀ acyl,
in particular R₂ and R′₂ represent 2- or 4-methoxyphenyl, 2- or 4-methylphenyl, phenyl, 2,4-dimethoxyphenyl,
and when n=0 and v=1, R₃ is then bound directly to the carbon bearing R₂ and R′₂,
or,
R₂ and R′₂ together represent, if n=0 and v=0, the ring of the following Formula (Va): 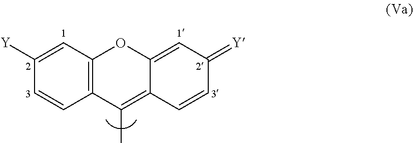
in which Y′ represents:
O,
NR_{d}Rₑ where R_{d} and Rₑ represent, independently of one another, H, a C₁-C₂₀ alkyl, the alkyl being able to form a ring with carbon 1′ or 3′, a C₃-C₂₀ cycloalkyl, the nitrogen atom having a positive charge associated with a monovalent anion,
and Y represents
OR′, where R′ represents H, a C₁-C₂₀ alkyl, a C₃-C₂₀ cycloalkyl,
a C₁-C₂₀ alkyl, a C₃-C₂₀ cycloalkyl,
a C₁-C₂₀ alkoxy,
NR_{f}R_{g} where R_{f} and Rg represent, independently of one another, H, a C₁-C₂₀ alkyl, the alkyl being able to form a ring with carbon 1 or 3, a C₃-C₂₀ cycloalkyl,
NO₂,
CO₂Rc, where Rc represents H, a C₂-C₂₀ alkyl, a C₃-C₂₀ cycloalkyl, a substituted or unsubstituted benzyl,
a C₁-C₂₀ acyl,
or, if n=0 and v=0, the ring of the following Formula (Vaa): 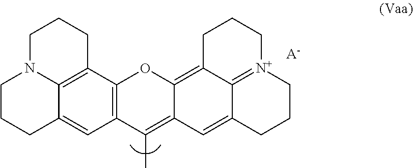
in which A⁻ represents a monovalent anion.
or
R₂ and R′₂ together represent the ring of the following Formula (Vb), n=1 and v=1: 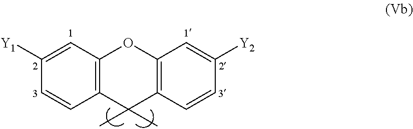
and Y₂ and Y₂ represent, independently of one another:
OR′, where R′ represents H, a C₁-C₂₀ alkyl, a C₃-C₂₀ cycloalkyl,
a C₁-C₂₀ alkyl, a C₃-C₂₀ cycloalkyl,
a C₁-C₂₀ alkoxy,
NRₕRᵢ where Rₕ and Rᵢ represent, independently of one another, H, a C₁-C₂₀ alkyl, the alkyl being able to form a ring with carbon 1 or 3 in the case of Y₁, and carbon 1′ or 3′ in the case of Y₂, a C₃-C₂₀ cycloalkyl,
NO₂,
CO₂Rc, where Rc represents H, a C₁-C₂₀ alkyl, a C₃-C₂₀ cycloalkyl, a substituted or unsubstituted benzyl,
a C₁-C₂₀ acyl,
or the ring of the following Formula (Vbb) and n=1: 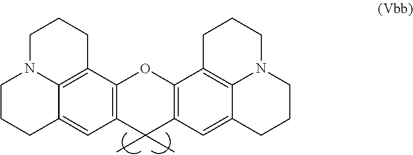 [SMILES: CC1(C)c2cc3c4c(c2Oc2c1cc1c5c2CCCN5CCC1)CCCN4CCC3]
R₃ represents an active ingredient, in particular an epigenetic modulator, a detecting probe, in particular fluorescent or radio-emitting, or a cell-penetrating peptide (CPP).

2. Nanovectors according to claim 1, in which the monomer units are derived from the polymerization of monocyclic alkenes, and are of the following Formula (Z1)
═[CH—R₅—CH]═ (Z1)
in which R₅ represents a saturated or unsaturated hydrocarbon chain with 2 to 10 carbon atoms.

3. Nanovectors according to claim 1, in which the monocyclic alkenes from which the monomer units originated are:
cyclobutene, leading to a polymer comprising monomer units of the following Formula (Z1a): 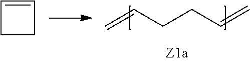
cyclopentene, leading to a polymer comprising monomer units of the following Formula (Z1b): 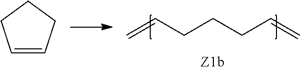
cyclopentadiene, leading to a polymer comprising monomer units of the Following formula (Z1c) 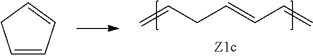
cyclohexene, leading to a polymer comprising monomer units of the following Formula (Z1d) 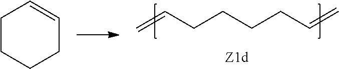
cyclohexadiene, leading to a polymer comprising monomer units of the following Formula (Z1e) 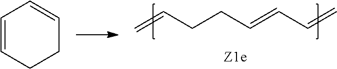
Z1e
cycloheptene, leading to a polymer comprising monomer units of the following Formula (Z1f) 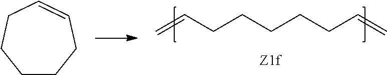
cyclooctene, leading to a polymer comprising monomer units of the following Formula (Z1h) 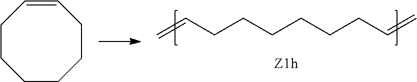
cyclooctapolyene, in particular cycloocta-1,5-diene, leading to a polymer comprising monomer units of the following Formula (Z1i) 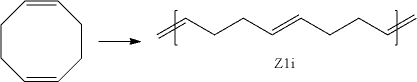
cyclononene, leading to a polymer comprising monomer units of the following Formula (Z1j) 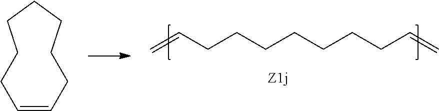
cyclononadiene, leading to a polymer comprising monomer units of the following Formula (Z1k) 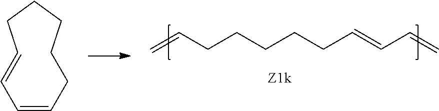
cyclodecene, leading to a polymer comprising monomer units of the following Formula (Z1l) 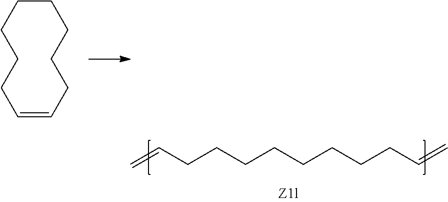
cyclodeca-1,5-diene, leading to a polymer comprising monomer units of the following Formula (Z1m) 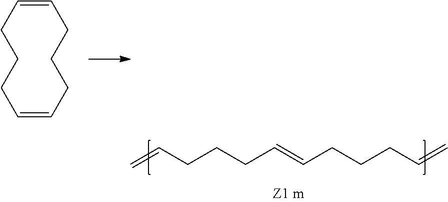
cyclododecene, leading to a polymer comprising monomer units of the following Formula (Z1n) 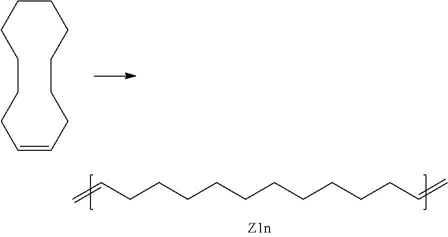
or also 2,3,4,5-tetrahydrooxepin-2-yl acetate, cyclopentadecene, paracyclophane, ferrocenophane.

4. Nanovectors according to claim 1, in which the monomer units are derived from the polymerization of polycyclic alkenes, and are:
of the following Formula (Z2)
═[CH—R₆—CH]═ (Z2)
in which R₆ represents:
* a ring of Formula 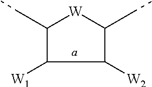
in which:
W represents —CH₂—, or a heteroatom, or a-CHR₇-group, or a-CHR₈— group, R₇ representing a chain comprising a poly(ethylene oxide) of Formula —(CH₂—CH₂—O)ₘ, m being as defined above and R₈ representing a C₁ to C₁₀ alkyl or alkoxy chain,
W₁ and W₂, independently of one another, represent H, or an R₇ chain, or an R₈ group mentioned above, or form, in combination with the carbon atoms bearing them, a ring of 4 to 8 carbon atoms, this ring being if appropriate substituted by an R₇ chain or an R₈ group mentioned above,
“a” represents a single or double bond,
* or a ring of Formula 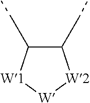
in which:
W′ represents —CH₂—, or a heteroatom, or a —CHR₇-group, or a —CHR₈— group, R₇ and R₈ being as defined above,
W′₁ and W′₂, independently of one another, represent —CH₂—, or a —C(O) group, or a —COR₈ group, or a —C—OR₈ group, R₇ and R₈ being as defined above,
of the following Formula (Z3) 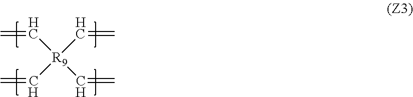
in which R₉ represents:
* a ring of Formula 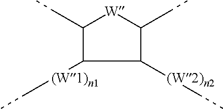
in which:
n₁ and n₂, independently of one another, represent 0 or 1,
W″ represents —CH₂—, or a —CHR— group, or a —CHR₈— group, R₇ and R₈ being as defined above,
W″₁ and W″₂, independently of one another, represent a hydrocarbon chain with 0 to 10 carbon atoms,
* or a ring of Formula 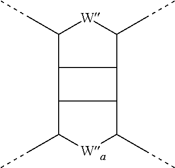
in which W″ and W″ₐ, independently of one another, represent —CH₂—, or a —CHR₇— group, or a —CHR₈— group, R₇ and R₈ being as defined above,
* or a ring of Formula 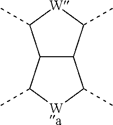
in which W″ and W″ₐ, independently of one another, represent —CH₂—, or a —CHR₇— group, or a —CHR₈— group, R₇ and R₈ being as defined above.

5. Nanovectors according to claim 1, in which the polycyclic alkenes from which the monomer units originated are:
the monomers containing a cyclobutene ring, leading to a polymer comprising monomer units of the following Formula (Z2a): 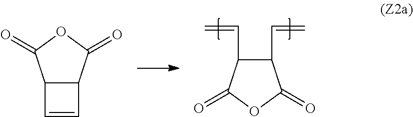
the monomers containing a cyclopentene ring, leading to a polymer comprising monomer units of the following Formula (Z2b): 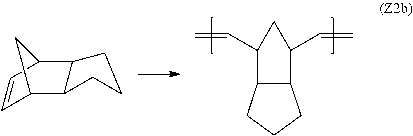
norbornene (bicyclo[2,2,1]hept-2-ene), leading to a polymer comprising monomer units of the following Formula (Z2c): 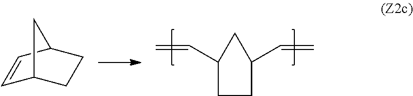
norbornadiene, leading to a polymer comprising monomer units of the following Formula (Z2d): 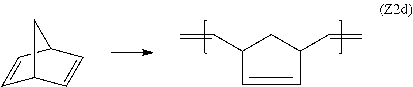
7-oxanorbornene, leading to a polymer comprising monomer units of the following Formula (Z2e): 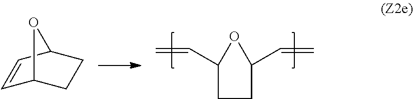
7-oxanorbornadiene, leading to a polymer comprising monomer units of the following Formula (Z2f): 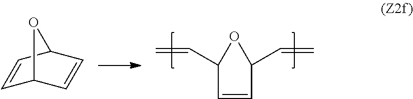
the norbornadiene dimer, leading to a polymer comprising monomer units of the following Formula (Z3a): 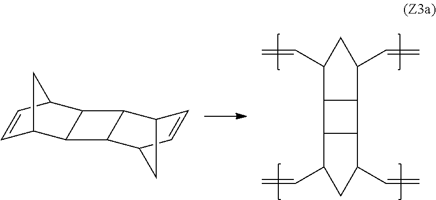
dicyclopentadiene, leading to a polymer comprising monomer units of the following Formula (Z3b): 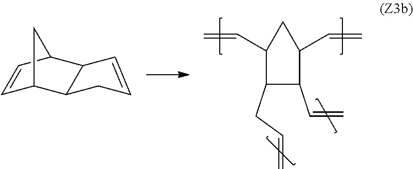
tetracyclododecadiene, leading to a polymer comprising monomer units of the following Formula (Z3c): 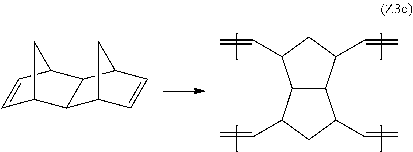
or bicyclo[5,1,0]oct-2-ene, bicyclo[6,1,0]non-4-ene.

6. Nanovectors according to claim 1, in which the mono- or polycyclic alkenes from which the monomer units originated are:
norbornene (bicyclo[2,2,1]hept-2-ene), leading to a polymer comprising monomer units of Formula (Z2c),
tetracyclododecadiene, leading to a polymer comprising monomer units of the following Formula (Z3c),
dicyclopentadiene, leading to a polymer comprising monomer units of the following Formula (Z3b),
the norbornadiene dimer, leading to a polymer comprising monomer units of the following Formula (Z3a),
cycloocta-1,5-diene, leading to a polymer comprising monomer units of the following Formula (Z1i).

7. Nanovectors according to claim 1, in which the epigenetic modulator is selected from:
a nucleoside, in particular cytidine, uridine, adenosine, guanosine, thymidine or inosine,
the histone deacetylase inhibitors (HDI), in particular Zolinza® (SAHA), trichostatin A (TSA), valproic acid, MS-275 or CI-994, or
the DNA methyltransferase inhibitors (DNMTI), in particular 5-azacytidine, 5-aza-2′-deoxycytidine and zebularine.

8. Nanovectors according to claim 1, in which the detecting probe is selected from a fluorophore, in particular rhodamine B or fluorescein, the coumarins, in particular 7-hydroxy-4-methylcoumarin, the Bodipy dyes, Texas red, the cyanines, in particular CY3 or CY5, or a radio-emitting substance such as ⁹⁹Technetium in liganded form, or contrast agents for medical imaging such as the lanthanides (gadolinium).

9. Nanovectors according to claim 1, in which the cell-penetrating peptide is selected from the polylysines, the polyarginines, the imidazole-modified polylysines, or mimetics of polyglycines with a chain bearing a nitrogen-containing end group.

10. Nanovectors as defined in claim 1, for use as medicament and/or diagnostic agent.

11. Nanovectors according to claim 10, for use in the treatment and/or diagnosis of pathologies selected from neurological diseases, inflammatory processes, cancer and diseases of the blood.

12. Nanovectors according to claim 10, for use in the combination treatment of pathologies selected from neurological diseases, inflammatory processes, cancer and diseases of the blood.

13. Pharmaceutical composition comprising, as active ingredient, nanovectors as defined in claim 1, in combination with a pharmaceutically acceptable vehicle.

14. Pharmaceutical composition according to claim 13, in a form that can be administered by intravenous route at a unit dose from 5 mg to 500 mg.

15. Method for preparing nanovectors constituted by polymer chains of general formula (I) as defined in claim 1, comprising a step of ring-opening metathesis polymerization and a step of bioconjugation.

16. Method for preparing nanovectors constituted by polymer chains of general formula (I) in which R₁ is a group of general formula (II) according to claim 15, characterized in that the step of ring-opening metathesis polymerization is carried out prior to the step of bioconjugation.

17. Method for preparing nanovectors according to claim 15, in which the step of ring-opening metathesis polymerization is carried out prior to the step of bioconjugation, comprising the following steps:
a. Preparation of a compound of the following general formula (VI-a) comprising a monocyclic or polycyclic alkene and a nitride function: 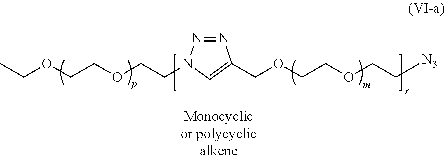
m, r and p being as previously defined,
b. Implementation of the step of ring-opening metathesis polymerization in the presence of a catalyst to form a compound of Formula (VII) comprising nitride functions on the surface of a polymer: 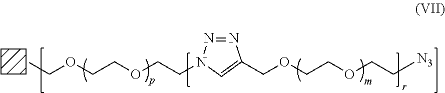
m, r, p and q being as previously defined,
c. Preparation of a compound of general formula (VIII) comprising an alkyne function: 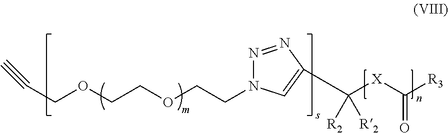
in which n, m, R₂, R′₂ and R₃ are as previously defined and s is an integer in the range from 0 to 10, in particular 0 or 1,
d. Implementation of the bioconjugation step by bringing said compound of Formula (VII) into contact with the compound of Formula (VIII) in the presence of copper to obtain nanovectors constituted by a polymer chain of Formula (I) in which R₁ is a group of Formula (II).

18. Method for preparing nanovectors constituted by polymer chains of general formula (I) in which R₁ is a group of general formula (II) and t=1, or of general formula (III) and t=0, according to claim 15, in which the step of bioconjugation is carried out prior to the optional step of ring-opening metathesis polymerization.

19. Method of preparation according to claim 18, comprising the following steps:
a. Preparation of a compound of the following general formula (VI-a) comprising a monocyclic or polycyclic alkene and a nitride function: 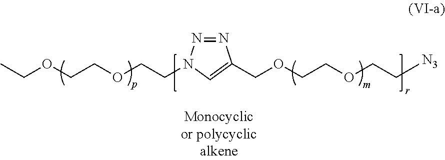
m, r and p being as previously defined,
b. Preparation of a compound of general formula (VIII) comprising an alkyne function: 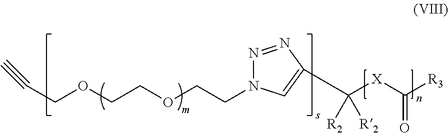
in which n, m, R₂, R′₂ and R₃ are as previously defined and s is an integer in the range from 0 to 10,
c. Implementation of the bioconjugation step by bringing said compound of Formula (VI-a) into contact with the compound of Formula (VIII) in the presence of copper to obtain the compounds of general formula (I) in which t=0 and R₁ represents a group of formula (III).
d. Optionally, implementation of the step of ring-opening metathesis polymerization in the presence of a catalyst to form nanovectors constituted by polymer chains of general formula (I) in which R₁ is a group of general formula (II) and t=1.
